(19) United States
(12) Patent Application Publication
(10) Pub. No.: US 2023/0395255 A1
(43) Pub. Date: Dec. 7, 2023

(54) METHODS FOR IDENTIFYING AND TARGETING THE MOLECULAR SUBTYPES OF ALZHEIMER'S DISEASE

(71) Applicant: Icahn School of Medicine at Mount Sinai, New York, NY (US)

(72) Inventors: Bin Zhang, New York, NY (US); Ryan A Neff, New York, NY (US); Vahram Haroutunian, New York, NY (US); Lap Ho, New York, NY (US); Xianxiao Zhou, New York, NY (US)

(73) Assignee: Icahn School of Medicine at Mount Sinai, New York, NY (US)

(21) Appl. No.: 18/250,206

(22) PCT Filed: Oct. 22, 2021
(86) PCT No.: PCT/US21/56315

§ 371 Date: Apr. 22, 2023

Related U.S. Application Data
(60) Provisional application No. 63/104,416, filed on Oct. 22, 2020.

(51) Int. Cl. G16H 50/20; G16B 40/20; G16H 20/10; G16B 20/00

(52) U.S. Cl. CPC G16H 50/20; G16B 40/20; G16H 20/10; G16B 20/00

(57) ABSTRACT

Disclosed herein are methods for identifying the neuronal and neurodegenerative phenotypes affected by and identified in Alzheimer's Disease, characterized as Alzheimer's Disease subtypes, methods for identifying drugs effective for treating Alzheimer's Disease subtypes, and drugs useful in treating Alzheimer's Disease subtypes.

### RELATED APPLICATION

This application is a § 371 national stage of PCT International Application No. PCT/US21/56315, entitled “Methods for Identifying and Targeting the Molecular Subtypes of Alzheimer's Disease” filed on Oct. 22, 2021, which claims priority to U.S. Provisional Application No. 63/104,416, filed Oct. 22, 2020, which is incorporated herein by reference in their entirety.

### STATEMENT REGARDING FEDERALLY SPONSORED RESEARCH

This invention was made with government support under U01AG046170 awarded by NIH on Sep. 30, 2018. The government has certain rights in the invention.

### BACKGROUND

Alzheimer's disease (“AD”) is the most common cause of dementia, an affliction that ultimately occurs in over 43 million people worldwide. The majority of dementia cases occur after age 65, which impose an increasing burden on societies with aging populations. AD is defined biologically by the presence of a specific neuropathology of the brain: extracellular deposition of amyloid-β (Aβ) in the form of diffuse and neuritic plaques and the presence of neuropil threads within dystrophic neurites that contain aggregated, hyperphosphorylated tau protein and intraneuronal neurofibrillary tangles (NFT) Both Aβ and NFT accumulation typically progress to targeted neuronal and synaptic loss, mainly in regions of the cerebral cortex and the hippocampus. Concurrent with the neuronal loss seen in AD, there is an additional coordinated breakdown across other brain cell types such as gliosis, demyelination, and inflammation which exacerbates cognitive dysfunction.

Despite the heavy burden on society and on aging populations, there are only four medications currently approved by the FDA for treating AD, and they are approved only for managing the cognitive impairment that are present in symptomatic AD. One explanation for the present paucity of effective AD therapies appears in recent developments indicating that AD is a heterogeneous disease caused by a variety of pathophysiologic mechanisms, mechanisms that often lie outside the current dogma regarding AD. For instance, up to one-third of patients with a clinical diagnosis of AD have no accumulation of amyloid-beta (Sekiya, et al., 2018), and many diagnosed with AD at post-mortem biopsy do not show cognitive impairment (Iaocono et al., 2014).

Sporadic Late-Onset Alzheimer's Disease (LOAD), the most prevalent form of dementia among people over age 65, is a progressive and irreversible brain disorder. Over 5.5 million in the US are affected by LOAD, which is currently the sixth leading cause of death in the US and costs more than $200 billion annually. There is an urgent need to develop effective methods to prevent, treat, or delay the onset or progression of LOAD. Among those at risk of LOAD, certain patients may carry a unique set of numerous genetic changes with greater risk for developing the disease, including CLU, TREM2, and most importantly Apolipoprotein E (APOE) variants (Lambert, et al. 2013). But the interaction between specific LOAD risk alleles, changes in disease pathogenesis, and their effects on patients, however, remains elusive.

Furthermore, it is very challenging to predict the progression of AD, suggesting high heterogeneity in disease progression among AD patients. There is growing evidence that disease progression and responses to interventions differ significantly within LOAD. For instance, patients with LOAD often branch into distinct groups including (a) slow vs. rapid cognitive decliners (Risacher et al., 2017); (b) amnestic vs. non-amnestic AD (Bredesen et al., 2015); (c) executive vs. cortical visual defect vs. dysphasia predominant AD (Phillips et al. 2018); (d) psychosis and/or depression associated AD (Qian et al. 2018); and (e) metabolic-dysfunction associated AD modulated by abnormalities in insulin resistance, hormonal deficiencies, or homocysteinemia (Huang et al. 2012). Finally, the relationship between the various forms of AD and other non-AD dementias such as primary age-related tauopathy (PART) (Crary et al., 2014), vascular contributions to cognitive impairment and dementia (VCID) (Chornenkyy et al. 2019), and frontotemporal dementia (Bang et al. 2015) must be better understood. Therefore, identifying unique molecular subtypes of AD resistant to other comorbid conditions may provide new insights into AD patient subpopulations and pave a way for precision medicine for AD.

Molecular biomarkers may hold the promise for improving methodologies for AD subtype identification and classification (Blalock et al. 2004; Courtney et al. 2010). Some recent studies have highlighted the great advantages of using RNA-seq to profile the transcriptome of the brain with neurodegenerative diseases. For instance, a multi-Omic molecular analysis of LOAD across four brain regions uncovered subnetworks and novel molecular drivers of the disease, including the vacuolar ATP-dependent proton pump ATP6V1A, which have now been shown to modulate cognitive function in Drosophila models of AD (Wang et al. 2019). Additionally, molecular network analysis of LOAD brains has identified an excess of dysregulated genes that cannot be fully predicted by a single model of the disease. Nevertheless, only a limited number of published papers describe RNA-seq studies of the most relevant materials, namely, AD patients' brains across multiple regions (Twine et al. 2011).

Thus, there is a need for characterizing the specific subtype signatures of AD, for identifying individual targets for treatment and for identifying drugs useful in the treatment.

### SUMMARY

The present disclosure overcomes the deficiencies noted above by identifying five molecular subtypes of AD and subsequently characterizing them with molecular signatures, network regulator genes, and matched mouse models. The identified subtypes are concentrated in the hippocampal area but distributed across brain regions.

The molecular AD subtypes identified in the present disclosure are well conserved across different independent cohorts, have independent molecular signatures, network regulator genes, and matched mouse models of AD.

Accordingly, these molecular subtypes can be used to: predict clinical features such as cognitive function or dementia; provide diagnostic signatures for classifying AD subtypes; identify key regulator genes across the subtypes and key genes unique to a given subtype; and provide methods for identifying new candidate drugs for treating AD and for stratifying patient populations for suitable AD treatments.

The present disclosure further provides methods for predicting such AD subtypes in affected subjects using whole genome sequencing. The present disclosure provides the first genomic copy number variation (CNV) study of LOAD based on whole genome sequencing data.

The present disclosure further provides methods for predicting AD subtypes in affected subjects using blood gene expression data.

The present disclosure also identifies FDA approved, investigational and experimental drugs that are useful in treating different AD subtypes.

### BRIEF DESCRIPTION OF THE DRAWINGS

FIG. 1 provides data revealing five different molecular subtypes of AD.

FIG. 2 provides mean values of several clinical and pathologic traits across AD subtypes.

FIG. 3 provides MEGENA and Bayesian causal network based key drivers of the AD subtypes.

FIG. 4 provides information showing cell type specific changes within each MSBB-AD subtype.

FIG. 5 provides information identifying the AD subtypes in the ROSMAP cohort.

FIG. 6 provides information matching existing AD Mouse Models to the MSBB-AD subtypes.

FIG. 7 provides information showing the genomic CNV distribution in the two cohorts (MSBB and ROSMAP).

FIG. 8 provides the overall features of the CNVs identified in MSBB and ROSMAP, including composition of CNV types, and site frequency spectrum (SFS).

FIG. 9 provides a comparison of the CNV sets in three clinical diagnostic groups (NL, MCI, and AD) in the two cohorts (MSBB and ROSMAP).

FIG. 10 provides a functional analysis of AD-, MCI-, and NL-specific CNV genes.

FIG. 11 provides predictions of AD subtypes based on blood monocyte transcriptomes.

### DETAILED DESCRIPTION

### Mechanisms and Therapeutic Targets for LOAD

The present disclosure provides for the first time a global landscape and detailed map of signaling circuits of complex molecular interactions in 4 key brain regions affected by LOAD, information that is critical for identifying specific treatment targets and identifying LOAD therapeutics. The present disclosure further provides multiple neuronal modules particularly relevant to LOAD pathology and predicts key regulators of these modules.

The present disclosure provides multiple AD subtypes and methods for effectively treating patients by correlating treatment methods with AD subtype.

The present disclosure provides methods of treating LOAD by administering to a subject in need thereof a therapeutically effective amount of an FDA approved drug.

The present disclosure provides a method of treating LOAD by administering to a subject in need thereof a therapeutically effective amount of a drug that targets individual AD subtypes.

The present disclosure provides methods for predicting AD subtypes based on whole genome sequencing alone.

The present disclosure provides methods for predicting AD subtypes using blood monocyte transcriptomes.

The present disclosure provides a method for treating Alzheimer's Disease (AD) in a patient in need thereof, wherein the method includes and administering a therapeutically effective amount of a compound selected from the group consisting of: thioproperazine; nalbuphine; gabexate; mesoridazine; dimercaptosuccinic-acid; menadione; carbamazepine; diphenidol; epirizole; timolol; mestranol; naphazoline; hesperidin; ethisterone; amlodipine; amsacrine; febuxostat; famciclovir; ezetimibe; carbetocin; orphenadrine; hyoscyamine; amiodarone.hcl; erythromycin-ethylsuccinate; meclizine; dobutamine; phenazopyridine; spironolactone; meclofenamic-acid; parachorophenol; bemegride; ketorolac; brinzolamide; nortriptyline; hexylcaine; omeprazole; norgestrel; olmesartan-medoxomil; perphenazine; promazine; metolazone; citalopram; clonazepam; lamotrigine; mosapride; and ephedrine.

The present disclosure provides a method for treating Alzheimer's Disease (AD) in a patient in need thereof, wherein the method includes and administering a therapeutically effective amount of a compound selected from the group consisting of: thioproperazine; nalbuphine; gabexate; mesoridazine; dimercaptosuccinic-acid; menadione; carbamazepine; diphenidol; epirizole; timolol; mestranol; naphazoline; hesperidin; ethisterone; amlodipine; amsacrine; febuxostat; famciclovir; ezetimibe; carbetocin; orphenadrine; hyoscyamine; amiodarone.hcl; erythromycin-ethylsuccinate; meclizine; dobutamine; phenazopyridine; spironolactone; meclofenamic-acid; parachorophenol; bemegride; ketorolac; brinzolamide; nortriptyline; hexylcaine; omeprazole; norgestrel; olmesartan-medoxomil; perphenazine; promazine; metolazone; citalopram; clonazepam; lamotrigine; mosapride; and ephedrine, and wherein the wherein the drug has been selected for treating at least one Alzheimer's Disease subtype.

The present disclosure provides a method for treating Alzheimer's Disease (AD) in a patient in need thereof, wherein the method includes and administering a therapeutically effective amount of a compound selected from the group consisting of: thioproperazine; nalbuphine; gabexate; mesoridazine; dimercaptosuccinic-acid; menadione; carbamazepine; diphenidol; epirizole; timolol; mestranol; naphazoline; hesperidin; ethisterone; amlodipine; amsacrine; febuxostat; famciclovir; ezetimibe; carbetocin; orphenadrine; hyoscyamine; amiodarone.hcl; erythromycin-ethylsuccinate; meclizine; dobutamine; phenazopyridine; spironolactone; meclofenamic-acid; parachorophenol; bemegride; ketorolac; brinzolamide; nortriptyline; hexylcaine; omeprazole; norgestrel; olmesartan-medoxomil; perphenazine; promazine; metolazone; citalopram; clonazepam; lamotrigine; mosapride; and ephedrine, and wherein the wherein the drug has been selected for treating at least one Alzheimer's Disease subtype selected from the group consisting of: AD subtype A, AD subtype B1, AD subtype B2, AD subtype C1, and AD subtype C2.

The present disclosure provides a method for treating Alzheimer's Disease (AD) in a patient in need thereof, wherein the method includes and administering a therapeutically effective amount of a compound selected from the group consisting of: GW-3965; ciglitazone; L-689560; fludroxycortide; sirtinol; Y-26763; mometasone-furoate; erythrosine; MDL-72832; NU-1025; cyclopentolate; ZM-306416; CP-93129; CGP-13501; CI-966; FK-888; PPT; cyclosporin-a; clofibric acid; neostigmine; FIT; ciproxifan; quinpirol-(−); clopidogrel; DMP-543; salmeterol; tremorine; piperidolate; pinacidil; erythrosine; mometasone-furoate; BML-284; GW-0742; adapalene; imatinib; CP-93129; proxyfan; tranylcypromine; ilomastat; FK-888; phenazopyridine; PNU-22394; clofibric acid; fenoterol; IBC-293; cyclopentolate; SQ-22536; UBP-296; atorvastatin; emetine; FH-535; altanserin; gamma-linolenic-acid; and alpha-linolenic-acid, and wherein the wherein the drug has been selected for treating at least one Alzheimer's Disease subtype selected from the group consisting of: AD subtype A, AD subtype B1, AD subtype B2, AD subtype C1, and AD subtype C2.

The present disclosure provides a computer-implemented method to predict an AD subtype of a subject, the method comprising: obtaining data of nucleotide characteristics for a sample collected from a particular human subject, wherein the sample is collected from the group consisting of: brain tissue; epithelial tissue; cerebrospinal fluid; and blood; providing the data as input to a trained machine learning model, wherein the model is selected from the group consisting of: Random Forest, hierarchical clustering, k-means clustering, WSCNA, MEGENA, Bayesian causal network, CNVnator, Pindel, MetaSV, Delly2, Quasipoisson regression, AdaBoost, logistic regression, decision tree, nearest neighbors (KNN), support vector machines (SVM), naïve Bayes, multi-layer perceptron, and Ensemble and wherein the model determines the AD subtype based on the data, wherein the AD subtype is any one of: AD subtype A, AD subtype B1, AD subtype B2, AD subtype C1, or AD subtype C2; and obtaining, from the model, the AD subtype.

The present disclosure provides a computer-implemented method to predict an AD subtype of a subject, the method comprising: obtaining data of nucleotide expression for a sample collected from a particular human subject, wherein the sample is collected from blood; providing the data as input to a trained machine learning model, wherein the model is selected from the group consisting of: Weighted Sample Gene Network Analysis (WSCNA) and Multi-scale Gene Expression Network Analysis (MEGENA); and wherein the model determines the AD subtype based on the data, wherein the AD subtype is any one of: AD subtype A, AD subtype B1, AD subtype B2, AD subtype C1, or AD subtype C2; and obtaining, from the model, the AD subtype.

The present disclosure provides a computer-implemented method to predict an AD subtype of a subject, the method comprising: obtaining data of nucleotide expression for a sample collected from a particular human subject, wherein the sample is collected from cerebrospinal fluid; providing the data as input to a trained machine learning model, wherein the model is selected from the group consisting of: Weighted Sample Gene Network Analysis (WSCNA) and Multi-scale Gene Expression Network Analysis (MEGENA); and wherein the model determines the AD subtype based on the data, wherein the AD subtype is any one of: AD subtype A, AD subtype B1, AD subtype B2, AD subtype C1, or AD subtype C2; and obtaining, from the model, the AD subtype.

The present disclosure provides a computer-implemented method to predict an AD subtype of a subject, the method comprising: obtaining data of gene expression levels for a sample collected from a particular human subject, wherein the sample is collected from blood; providing the data as input to a trained machine learning model, wherein the model is selected from the group consisting of: Random Forest, AdaBoost, logistic regression, decision tree, nearest neighbors (KNN), support vector machines (SVM), naïve Bayes, multi-layer perceptron, and an Ensemble with equal weights for each classifier and wherein the model determines the AD subtype based on the data; wherein the AD subtype is any one of: AD subtype A, AD subtype B1, AD subtype B2, AD subtype C1, or AD subtype C2; and obtaining, from the model, the predicted AD subtype.

The present disclosure provides a computer-implemented method to predict for identifying candidate compounds for use in treating an AD subtype, the method comprising: obtaining data of drug induced signatures for candidate compounds and AD subtype signatures, wherein the AD subtypes are selected from the group consisting of: AD subtype A, AD subtype B1, AD subtype B2, AD subtype C1, or AD subtype C2; providing the data as input to a trained machine learning model, wherein the model is EDMURA; and obtaining from the model, the drug associated with an AD subtype.

### Definitions

Unless defined otherwise, all technical and scientific terms used herein have the meaning commonly understood by a person skilled in the art to which this disclosure belongs. The meaning and scope of the terms should be clear, however, in the event of any latent ambiguity, definitions provided herein take precedent over any dictionary or extrinsic definition.

As used herein, the following terms have the meanings ascribed to them below, unless specified otherwise. The terminology used herein is for the purpose of describing particular embodiments only and is not intended to be limiting of the disclosure. As used herein, the singular forms “a”, “an” and “the” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “effective amount” or “therapeutically effective amount” refers to a quantity of a drug sufficient to achieve a desired effect or a desired therapeutic effect. In the context of therapeutic applications, the amount of the drug administered to the subject can depend on the type and severity of the disease or symptom and on the characteristics of the individual, such as general health, age, sex, body weight and tolerance to drugs. The skilled artisan will be able to determine appropriate dosages depending on these and other factors.

As used herein, the terms “treat,” “treatment,” or “treating” includes any treatment of a condition or disease in a subject, or particularly a human, and may include: (i) preventing the disease or condition from occurring in the subject which may be predisposed to the disease but has not yet been diagnosed as having it; (ii) inhibiting the disease or condition, i.e., arresting or slowing down its progression; relieving the disease or condition, i.e., causing regression of the condition; or (iii) ameliorating or relieving the conditions caused by the disease, i.e., symptoms of the disease. “Treat,” “treatment,” or “treating,” as used herein, could be used in combination with other standard therapies or alone.

As used herein, the term “late-onset Alzheimer's Disease” or “LOAD” includes patients who were diagnosed with AD at age 65 or older.

As used herein, the term “Alzheimer's Disease subtype” or “AD subtype” refers to a category of Alzheimer's Disease that is determined by a distinct molecular signature comprised of up- and down-regulated genes which can be further characterized by the presence or absence of particular pathways (markers), as provided in the present disclosure, and falling into one of the following categories: AD subtype A; AD subtype B1; AD subtype B2; AD subtype C1; and AD subtype C2.

As used herein, the term “AD subtype A” refers to the Alzheimer's Disease subtype provided in this disclosure as having specific traits, including, for example: opposite to the differential expression directions of the Blalock signatures; up regulation of the GNF2_MAPT pathway; upregulation of glutaminergic, GABAergic, and dendritic synaptic pathways; upregulation of protein degradation related genes, including ubiquitination and polyubiquitination, protein catabolism, the proteasome, and proteins targeting for destruction; upregulated neuronal regulators (GABRB2, SYT1, NSF, SLC4A10, SLC9A6 and SCN2A); downregulated KDGs in astrocytes, endothelial cells, and microglia (LRP10, NOTCH1, ITGB5, MYO1C, and TLN1).

As used herein, the term “AD subtype B1” refers to the Alzheimer's Disease subtype provided in this disclosure as having specific traits, including, for example: up regulation of the GNF2_MAPT pathway; upregulation of glutaminergic, GABAergic, glycinergic, and dendritic synaptic pathways; upregulation of organic acid related genes, including acid secretion and acidic amino acid transport; downregulation of genes in oligodendrocytes (PLP1, UGT8, CLDND1, ERMN, and ENPP2); upregulation of genes in neurons (CACNA1B, BSN, FBXO41, CHD5, DGKZ, SYT7, CELSR3 and RAPGEFL1); and upregulation of genes in astrocytes (IQSEC2).

As used herein, the term “AD subtype B2” refers to the Alzheimer's Disease subtype provided in this disclosure as having specific traits, including, for example: up regulation of the GNF2_MAPT pathway; upregulation of glutaminergic, GABAergic, glycinergic, and dendritic synaptic pathways; upregulation of innate and adaptive immune response, immune system activation, inflammation, circulatory system development, and endothelial cell migration; upregulation of organic acid related genes, including acid secretion and acidic amino acid transport; increased APOE e2 allele dosage; downregulation of genes in oligodendrocytes (PLP1, UGT8, CLDND1, ERMN, and ENPP2); downregulation of PICALM and of PSMC6; upregulation of FBXO41, WIZ, PRRC2A, ZMIZ2, CIC, and TCEA1.

As used herein, the term “AD subtype C1” refers to the Alzheimer's Disease subtype provided in this disclosure as having specific traits, including, for example: consistent with the Blalock signatures; upregulation of amyloid-beta binding, clearance, fiber formation pathways and of scavenger receptor activity; down regulation of the GNF2 MAPT pathway; downregulation of glutaminergic, GABAergic, glycinergic, and dendritic synaptic pathways; upregulation of innate and adaptive immune response, immune system activation, inflammation, circulatory system development, and endothelial cell migration; increased APOE e4 allele dosage; downregulation of GABRB2, SYT1, and PREPL; and KDGs are upregulated in microglia (TLN1, MSN, and IL6R), endothelial cells (TAGLN2), and astrocytes (LRP10, GNA12, and LTBP3) and downregulated in neurons (ATP6V1A, SCN2A, GABRB2, and NAPB); downregulation of AMPH, MEF2C, and EPDR1.

As used herein, the term “AD subtype C2” refers to the Alzheimer's Disease subtype provided in this disclosure as having specific traits, including, for example: consistent with the Blalock signatures; down regulation of the GNF2_MAPT pathway; downregulation of glutaminergic, GABAergic, glycinergic, and dendritic synaptic pathways; upregulation of innate and adaptive immune response, immune system activation, inflammation, circulatory system development, and endothelial cell migration; downregulation of GABRB2, SYT1, GABRB2, SCN2A, NSF, GABBR2, and PREPL; upregulation of STAT3, SLC39A1, LRP10, GNA12, TAGLN2, IL6R, and MAPKAPK2.

### EXAMPLES

The following examples are put forth to provide those of ordinary skill in the art with a complete disclosure and description of how to make and use the compositions, and assay, screening, and therapeutic methods of the invention, and are not intended to limit the scope of what the inventors regard as their invention.

### Example 1

### Parahippocampal Gyrus Molecular Signal of LOAD

Clinical and transcriptomic signatures from the MSBB-AD study of 364 human brains were obtained, including whole transcriptome RNA-seq data from four brain regions (FP, STG, PHG, IFG) from subjects with AD that showed neurocognitive decline as measured by clinical dementa rating (CDR) score>1 and non-demented controls (CDR=0). Table 1 summarizes the clinical and pathologic phenotypes for the samples in the MSBB-AD cohort with the transcriptomic data from the parahippocampal gyrus. The inventors identified numerous confounding factors in the RNA-seq data, including age of death, race, and post-mortem interval (PMI). To minimize re-identification of clinical and technical covariates, the transcriptomic data was corrected for age of death, race, gender, post-mortem interval (PMI), batch number, and RNA integrity number (RIN) using a mixed effects model.

To understand which brain regions and molecular processes are most vulnerable to dysregulation in AD, the inventors performed differential gene expression analysis between AD and control, generating differentially expressed genes (DEGs) for each of the four brain regions in the MSBB-AD. The inventors discovered that the PHG brain region has the largest number of DEGs (3571 genes, adjusted FDR=0.05) compared to the FP (3 genes), STG (1 gene), and IFG (181 genes). Their findings are consistent with previous DEG analyses of the MSBB-AD transcriptomic data and suggest that the PHG is most vulnerable in AD as manifested by marked transcriptomic dysregulation. Moreover, these findings are consistent with their previous pan-cortical atlas of AD (independent of the data described herein) in which the PHG brain region showed the strongest transcriptomic changes (Wang et al. 2016). Indeed, this prior work has shown that the hippocampus is strongly associated with A3 and tau accumulation and early memory loss in AD. The inventors have also shown in previous studies of the MSBB-AD cohort that transcriptomic changes in the PHG region highly overlap the KEGG Alzheimer's and Parkinson's disease gene sets and are correlated with high A3 plaque density (Wang et al. 2019), demonstrating that these changes are consistent with AD disease progression. Therefore, the present disclosure shows that the PHG region carries the strongest molecular signature of AD.

TABLE 1
Summary of Clinical and Pathologic Phenotypes for MSBB-AD
Samples with PHG Transcriptomic Data

| | | | P-value |
|---|---|---|---|
| Metric (mean +/− SD) | Control | AD | (t-test) |
| Number of subjects | 32 | 151 | — |
| % Female | 68.8% | 66.9% | 0.846 |
| % European ancestry | 71.9% | 81.9% | 0.209 |
| % African ancestry | 18.8% | 11.8% | 0.046 |
| % Hispanic ancestry | 6.2% | 3.9% | 0.572 |
| % Other ancestry | 0% | 2.4% | 0.38 |
| Age of death (years) | 82.8 y +/− 10.1 | 86.3 y +/− 8.9 | 0.0479 |
| Clinical dementia | 0 +/− 0 | 3.4 +/− 1.1 | 5.6*10⁻³⁴ |
| rating (CDR) | | | |
| Mean Abeta plaque | 2.1 +/− 3.1 | 11.6 +/− 9.8 | 2.2*10⁻⁷ |
| number (per field) | | | |
| Pathology + CDR | 2.16 +/− 1.27 | 4.16 +/− 2.08 | 6.22*10⁻⁷ |
| composite | | | |
| (“Brain Bank score”) | | | |
| Post-mortem interval | 546 +/− 343 min | 368 +/− 270 min | 1.9*10⁻³ |

### Example 2

### Normalization of Data by AD Stages Removes Confounding Signal of Neuronal Loss

Patients with more severe AD-associated dementia, such as those in a later stage of the disease, are reported to have more neuronal loss at post-mortem biopsy. Therefore, it is important to control for AD stage before transcriptomic analysis is performed between subjects with AD. Previous work has shown that brain cell type proportions (SPVs), including the proportion of neurons in a sample, can be inferred from bulk RNAseq data when combined with measurements of brain cell-type specific gene expression patterns (McKenzie et al. 2018), and may serve as a marker of neuronal loss in AD.

To determine the extent of hippocampal neuronal loss in the MSBB-AD cohort, cell type proportion analysis of bulk tissue transcriptomic data from the PHG region was performed. The inventors discovered a strong relationship between PHG neuronal loss and clinical dementia rating (CDR) score in the MSBB-AD cohort, with astrogliosis and increased abundance of other cell types associated with disease progression. Currently, there is no universally accepted method to rectify for neuronal loss seen in AD, as this reduction in neurons is both the cause and effect of molecular changes leading to cognitive impairment. The inventors examined both normalization by neuronal cell type proportion SPVs and AD dementia (as measured by CDR score, range 0-6) to reduce the molecular signature of neuronal loss in AD, using a linear effect model. The inventors found that there is no significant correlation between cell type and AD staging after either normalization by neuronal cell type proportion or by dementia severity. Furthermore, the inventors did not see a further reduction in this correlation with additional neuronal cell type normalization after normalization by dementia severity. Thus, the inventors found that both normalization by CDR and cell-type proportion SPVs are effective in removing the confounding effects of neuronal loss along disease progression in the MSBB-AD cohort.

The inventors further normalize the PHG transcriptomic data by CDR score, to remove the confounding effects of neuronal loss in later AD stages. Therefore, any identified differences between groups of AD subjects would be distinct from previously identified clinical subtypes of AD which rely on these metrics.

### Example 3

Identification of AD Putative Subtypes from Molecular Data of the PHG

To robustly identify subgroups of AD subjects, the inventors evaluated the performance of several sample clustering methods for AD subjects using by the normalized gene expression data within each brain region in the MSBB-AD cohort. The inventors used two classical clustering algorithms (hierarchical, k-means) as well as two novel network-based clustering algorithms (Weighted Sample Gene Network Analysis (WSCNA), Multi-scale Gene Expression Network Analysis (MEGENA)) to group similar samples together into putative AD subtypes. WSCNA shows the best performance in terms of clustering quality and thus is adopted to identify AD subtypes for the subsequent analyses.

The inventors successfully identified clusters of related samples using all four methods for each of the four brain regions. To determine the likelihood that these sample clusters may represent molecular subtypes of AD and that the sample grouping is consistent, 50 rounds of bootstrapped reclustering were performed using each clustering algorithm while withholding 20% of the samples and genes per round. An empirical calculation was then performed to determine the likelihood that samples are consistently grouped together from the observed clusters compared with a distribution of 100,000 possible random groupings. A specific subtype grouping is considered a putative subtype if its empirically adjusted p-value is less than 0.05. Employing this method, the inventors detected the presence of putative AD subtypes using all four clustering methods (emp. p-value: <0.05) based on molecular data from the PHG region alone. Among the four algorithms evaluated, the inventors found that their new network-based clustering approach, WSCNA, shows the highest likelihood of stable subtypes compared to random clustering of 7.08:1 (emp. p-value: <1*10⁻⁵) in the PHG region. The inventors found that the other clustering methods also identified subtypes in the PHG, but with a smaller likelihood ratio. Furthermore, among all four brain regions in the MSBB-AD, the inventors discovered that the PHG region shows the most robust AD subtype signal.

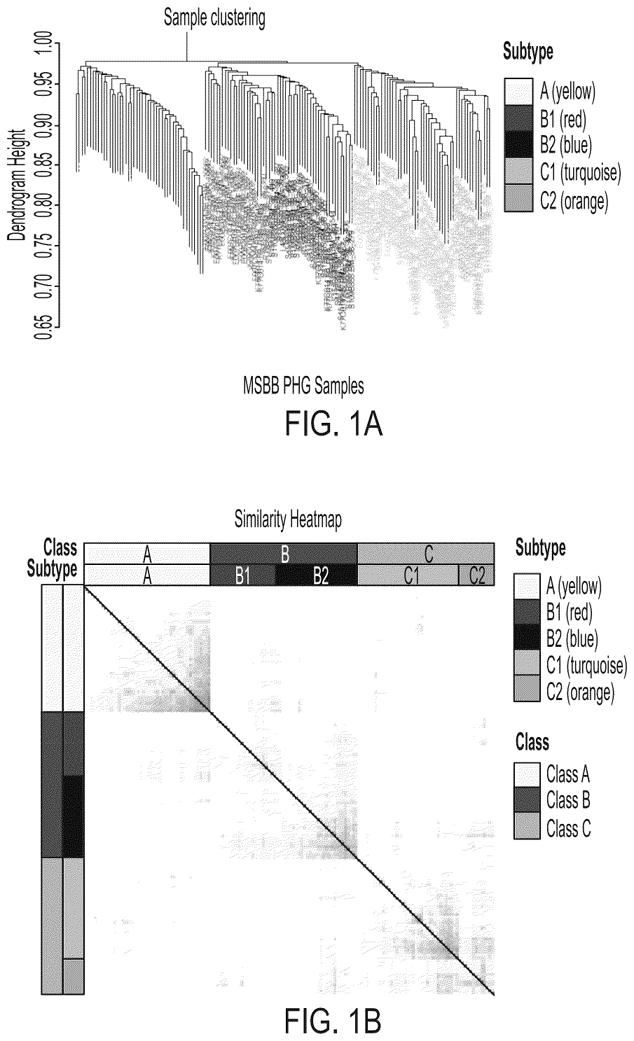

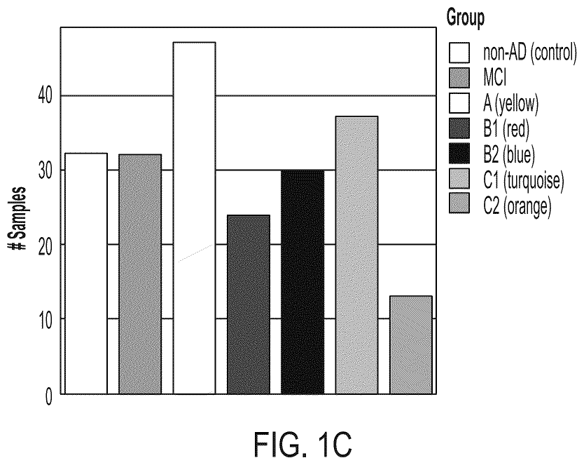

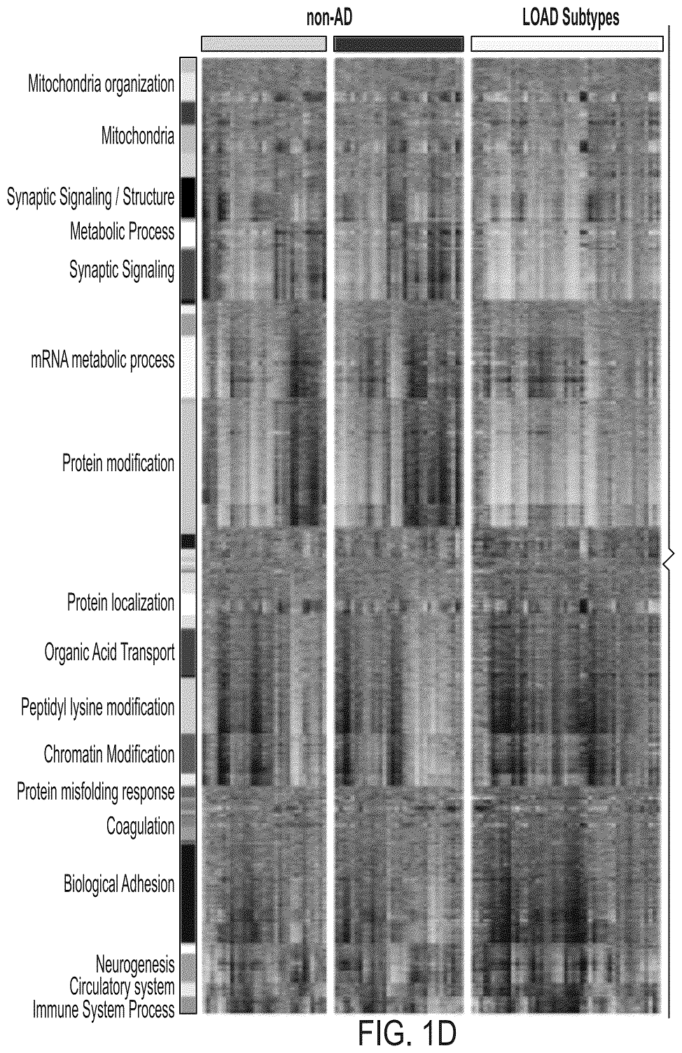

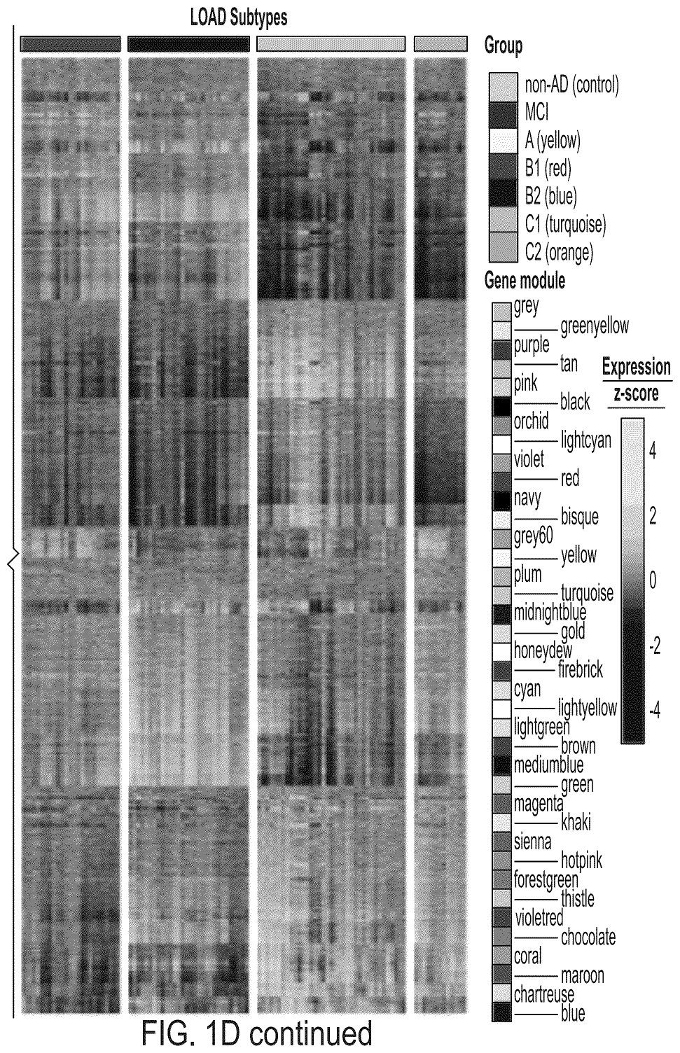

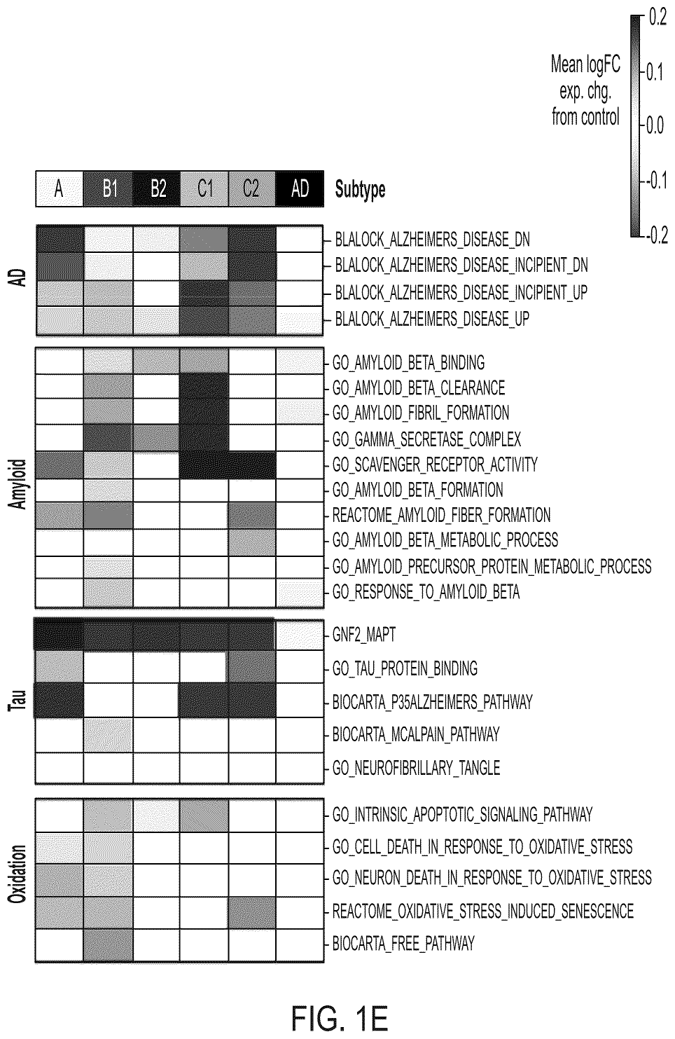

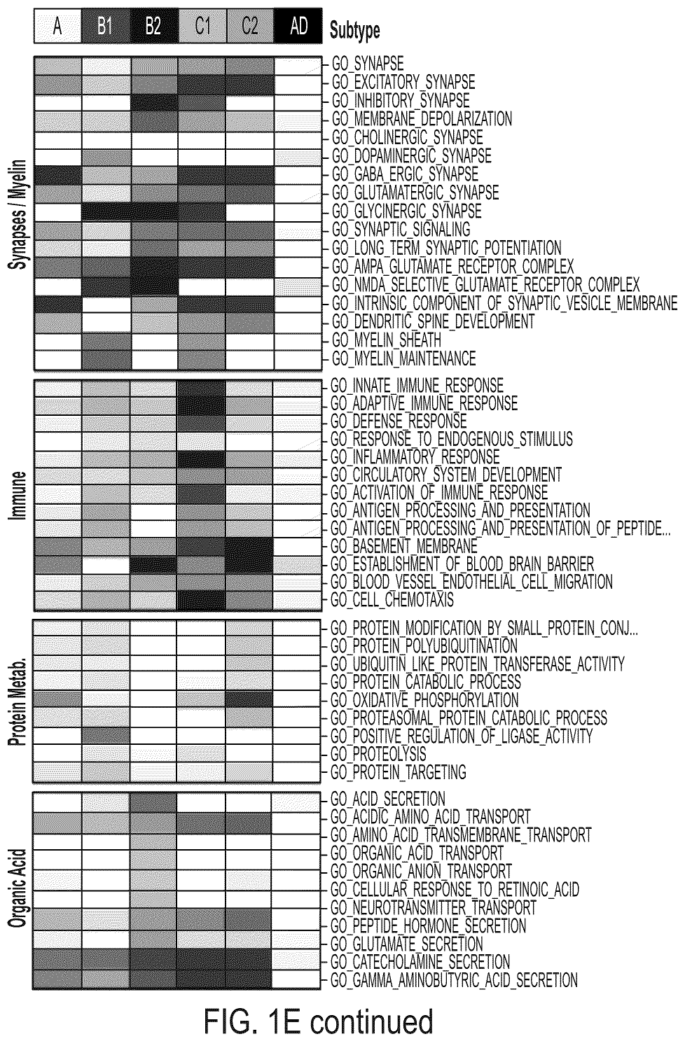

As shown in FIG. 1, the inventors found that their WSCNA algorithm identifies five subtypes in the MSBB-AD (clusters A, B1, B2, C1, C2) across all 151 subjects with PHG transcriptomic data. Based on the dendrogram and the network similarity heatmap (FIG. 1a-b), the inventors were able to further group the five subtypes into three major classes of AD labeled class A, class B (comprising subtypes B1 and B2), and class C (comprising subtypes C1 and C2). Each class of subtypes has a similar number of samples (47 in class A, 54 in class B, and 50 in class C) (FIG. 1c).

Cluster stability is defined here as the rate at which sample pairs group together into the same subtypes upon repeated re-clustering on a random subset of the input data. Subtypes from WSCNA clustering are generally stable, and sample pairs grouped together, on average, between 60-91% across all five detected AD subtypes. The class C subtypes have the strongest stability, followed by class A and class B. All subtypes demonstrate a cluster stability strongly above random clusters, which was empirically determined at a stability range between 20-30%. Therefore, the subtypes found by the inventors show specific robust molecular signals suitable for classification into stable subtypes.

### Example 4

### Molecular Signatures of Putative AD Subtype

To characterize the molecular signatures of these AD subtypes, the inventors identified DEGs for each of the five subtypes compared with non-demented controls (CDR=0) from the RNA-seq data in the PHG region. The inventors found that each AD of the subtypes they discovered has a specific transcriptomic signature of up- and down-regulated genes that distinguishes it from the others at a molecular level, revealing a plurality of different mechanisms of AD. As shown by FIG. 1d, there is a clear separation of molecular signatures between the five AD subtypes that can be visually appreciated from the whole transcriptome gene expression heatmap, after identifying gene modules using weighted interaction network analysis (WINA).

Using mean gene expression levels grouped by gene ontology (GO) pathway as surrogate markers for the activity level of various molecular processes in the brain, the inventors identified several differences in key AD-related pathways between the subtypes, providing key insights into disease pathogenesis. As shown in FIG. 1e, the inventors found significant deviations (Welsh's p-value<0.05) in 74 AD-related signatures from previous studies and GO pathways, including pathways related to AD, oxidative stress, tau NFT, and synaptic function, across the five AD subtypes and controls. First, the inventors compared the subtype molecular signatures that they identified with the post-mortem hippocampal transcriptional signatures of AD identified by Blalock et al., (termed Blalock signatures). Overall, molecular signatures from all AD subjects are consistent with Blalock; however, this consistency is not shared between each of the individual AD subtypes identified by the inventors. The inventors found that the direction of the gene expression changes in the class C subtypes is consistent with the Blalock signatures, while the changes in the class A subtype are opposite to Blalock. In contrast, the inventors found that the signature of class B subtypes do not show significant enrichment of the Blalock signatures. Therefore, AD subtypes may be classified into three larger classes (classes A, B, and C), i.e., typical (class C), intermediate (class B), or atypical (class A), by molecular presentation when compared to the Blalock signatures of AD.

Surprisingly, the inventors observed only weak molecular enrichment of amyloid-beta and tau related pathways across all AD subjects as a group, but they saw strong enrichment of these pathways in the subtypes. For instance, the inventors discovered strong upregulation of amyloid-beta binding, clearance, and fiber formation pathways in the subtype C1, as well as scavenger receptor activity in the subtypes C1 and C2, while these same pathways are down-regulated in the subtype B1 and mild downregulation in the subtype A. On the other hand, they found that tau-neighborhood genes (“GNF2_MAPT” pathway) are strongly up-regulated in the subtypes A, B1, and B2 but downregulated in C1 and C2. Tau protein binding and tau-related P35 pathway genes are up-regulated in the subtype A. Therefore, it is likely that AD subtypes may be characterized by either amyloid-beta activity predominant (class C) or MAPT-activity predominant (class A+B) though they cannot fully explain all differences seen between the five subtypes.

The subtypes identified by the inventors also differ strongly in neuronal activity despite normalization for AD staging. The inventors have discovered broad downregulation of glutaminergic, GABAergic, glycinergic, and dendritic synaptic pathways in class C subtypes, with absent changes in cholinergic and dopaminergic synaptic pathways, suggesting that these synapse types are selectively resilient to AD subtype molecular changes. On the other hand, the inventors found strong upregulation of these same synapse pathways in the classes A and B, with the exception of upregulation of glycinergic synapse in the class A. This pattern is consistent with differences in synaptic excitation pathways between subtypes: excitatory synapses are up-regulated in the classes A and B but down-regulated in the class C. These data suggest that AD subtypes may be split into those selectively vulnerable to synaptic depression (class C) versus synaptic excitation (classes A and B).

Dysregulated immune system activities, including reactive gliosis and the breakdown of the blood-brain barrier, have been repeatedly observed in AD brains (Sweeney et al. 2018). The present disclosure shows that the subtypes B2, C2, and especially C1, immune related pathways including the innate and adaptive immune response, immune system activation, inflammation, circulatory system development, and endothelial cell migration are upregulated in comparison with the normal control. Such upregulation coincides with increased expression of blood-brain barrier, basement membrane, and cell matrix adhesion genes. However, these immune response pathways are down regulated in the subtypes A and B1. These data and the findings relative to synaptic pathways suggest that disease progression across AD subtypes is characterized by either increased immune or synapse pathway activity.

Finally, the inventors found that certain molecular pathways are subtype-specific and thus provide greater insights into disease pathogenesis when considering other enriched AD pathways. For example, the present disclosure shows that many protein degradation related genes, including ubiquitination and polyubiquitination, protein catabolism, the proteasome, and proteins targeting for destruction are up-regulated in the subtype A, while organic acid related genes, including acid secretion and acidic amino acid transport, are specifically up-regulated in the class B.

### Example 5

Association of Clinical and Pathological Phenotypes and APOE Variants with Putative AD Subtypes

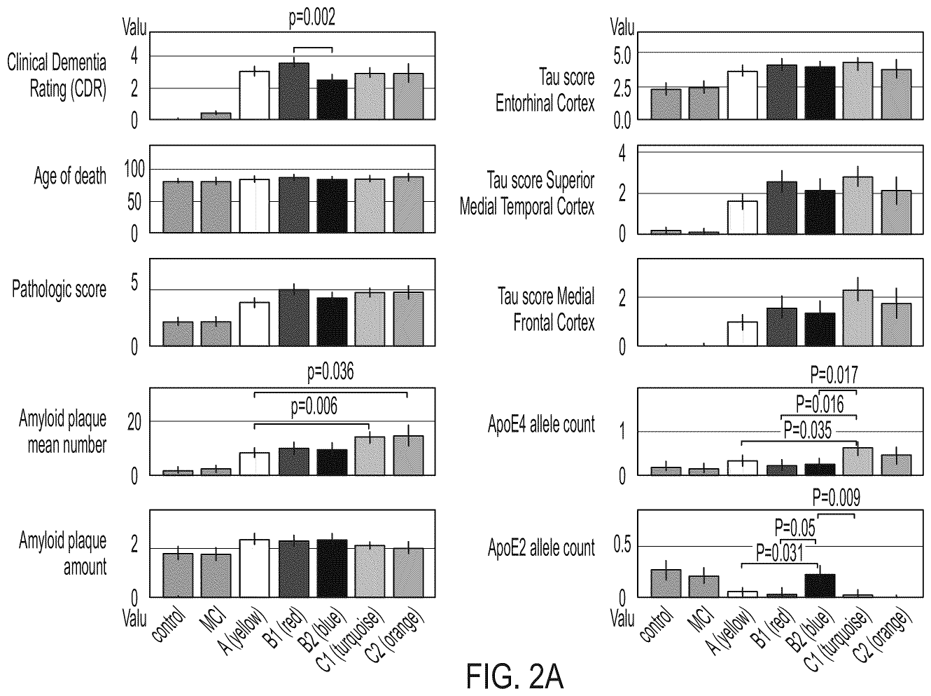

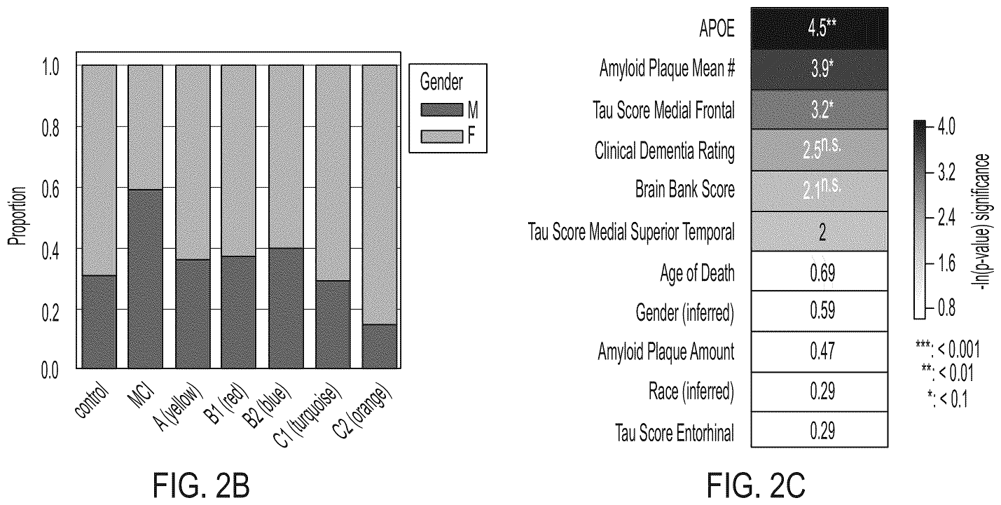

The present disclosure provides a better understanding of the clinical characteristics of these molecularly defined AD subtypes, by comparing the relationship between characterized AD pathologic markers in the MSBB-AD study and each subtype. The inventors found that under the Kruskal-Wallis (KW) one-way analysis of variance test, AD subtypes are marginally associated with several clinical AD markers, including tau NFT levels in the medial frontal cortex (KW p=0.041), Aβ mean plaque levels (KW p=0.020), and APOE e4 (KW p=0.048) and APOE e2 (KW p=0.012) allele counts (FIG. 2b-c). They also found that the “amyloid-beta predominant” AD subtypes (class C), with a mean plaque number of 14.2/mm², show a significantly larger amyloid plaque burden than both of the “MAPT-predominant” class A (mean=8.4/mm², Welsh's t-test p=3.1*10⁻³) and the class B (mean=9.6/mm², Welsh's t-test p=0.018) subtypes, despite no significant difference in cognitive decline as measured by CDR. Consistent with the preprocessing steps already performed on the PHG data, the subtypes do not show significant difference in previously corrected covariates. The inventors did not see significant changes in CDR score (KW p=0.082, FIG. 2c), biological sex (KW p=0.554, FIG. 2b-c), ethnicity (KW p=0.748), post-mortem interval (KW p=0.502), or age of death (KW p=0.503) across AD subtypes.

The present disclosure provides a better understanding of the differences in APOE allele dosages between AD subtypes. The inventors found that certain subtypes are preferentially enriched or depleted for the e4 and e2 alleles compared with others. For instance, the subtype C1 has a significantly increased APOE e4 allele dosage (median: 0.61 alleles/pt) compared with the subtypes A (p=0.035 under Welsh's t-test), B1 (p=0.015), and B2 (p=0.017). This is consistent with the known influence of the e4 allele on AD pathogenesis, including the formation of amyloid-beta plaques and NFTs, a trait most similar to the molecular signature of the amyloid-predominant subtypes. On the other hand, subtype C2, which shares many molecular features with C1, does not show this association with APOE e4 in the present disclosure. Furthermore, subtype B2 has an increased APOE e2 allele dosage (median: 0.23 alleles/pt) compared with subtype A (p=0.031) and C1 (p=0.0091); however, like APOE e4 among the class C subtypes, the APOE e2 dosage is also much higher than subtype B1 (p=0.049). Therefore, while APOE may modulate AD pathogenesis and contribute to some molecular signatures in a portion of subtypes, the present disclosure shows that APOE dosage cannot explain all the molecular similarities and differences between both related and distinct AD subtypes.

The present disclosure shows that a subset of post-mortem Alzheimer's brains with PHG transcriptomic data available (n=55 out of 151) have additional quantification of amyloid-beta plaque and tau NFT amounts across multiple brain regions. The inventors found that tau NFT counts are significantly associated with the AD subtypes across the inferior parietal lobule (KW test p=0.017) and medial frontal gyrus (KW p=0.034). In these regions, both the class B and C subtypes have increased tau NFT burden. In contrast, the inventors found that amyloid-beta plaque rating is significantly elevated in the inferior parietal lobule (KW p=0.031), medial frontal gyrus (KW p=0.041), and lateral frontal gyrus (KW p=0.012) in only the class C (amyloid-predominant) subtypes. These discoveries are consistent with the previous signatures from the GO pathway analysis, indicating that class C subtypes are amyloid-beta predominant, while class B subtypes are tau NFT predominant. While class A shows increased MAPT pathway activity, it is resilient to the development of tau NFTs, perhaps via increased protein degradation pathway activity. Therefore, these disclosures indicate that class A subtypes are tau NFT resilient. As the inventors expected from the analysis on all samples, the present disclosure shows that both CDR (KW p=0.155) and Braak score (KW p=0.075) are not associated with the AD subtypes. Therefore, the present disclosure shows that AD staging is not associated with the changes in amyloid-beta plaque and tau NFT levels in the subtypes.

### Key Network Regulators of AD Subtypes

The diverse molecular changes that the inventors have identified in the AD subtypes suggest distinct intrinsic molecular mechanisms underlying each subtype. To identify the key regulators of the molecular changes in each AD subtype, the inventors employed a network biology approach that integrates multiscale embedded gene co-expression network analysis (MEGENA) and Bayesian causal network (BN) inference. Towards this end, the inventors constructed a co-expression network based on all the AD samples in the PHG which includes 22,291 genes and 61,152 edges, and a Bayesian causal network comprised of 21,577 genes and 23,554 edges. The inventors performed key driver analysis of each resulting network and the subtype DEG signatures and identified a ranked list of 955 upregulated and 639 downregulated key network regulator genes (KNRs) in the MEGENA network and a ranked list of 1,226 upregulated and 846 downregulated KNRs in the BN network. Finally, the intersection of the BN and MEGENA network KNRs yields a subset of 233 up- and 164 down-regulated KNRs across the five subtypes (Table 2).

TABLE 2

| | # MEGENA | # MEGENA | # BN | # BN | # Overlap | # Overlap |
|---|---|---|---|---|---|---|
| | key reg. genes | key reg. genes | key reg. genes | key reg. genes | key reg. genes | key reg. genes |
| Subtype | upregulated | downregulated | upregulated | downregulated | upregulated | downregulated |
| B2 | 388 | 165 | 165 | 76 | 101 | 34 |
| B1 | 94 | 66 | 81 | 26 | 26 | 17 |
| A | 121 | 82 | 73 | 24 | 43 | 11 |
| C1 | 287 | 225 | 107 | 189 | 65 | 78 |
| C2 | 336 | 308 | 95 | 124 | 55 | 87 |

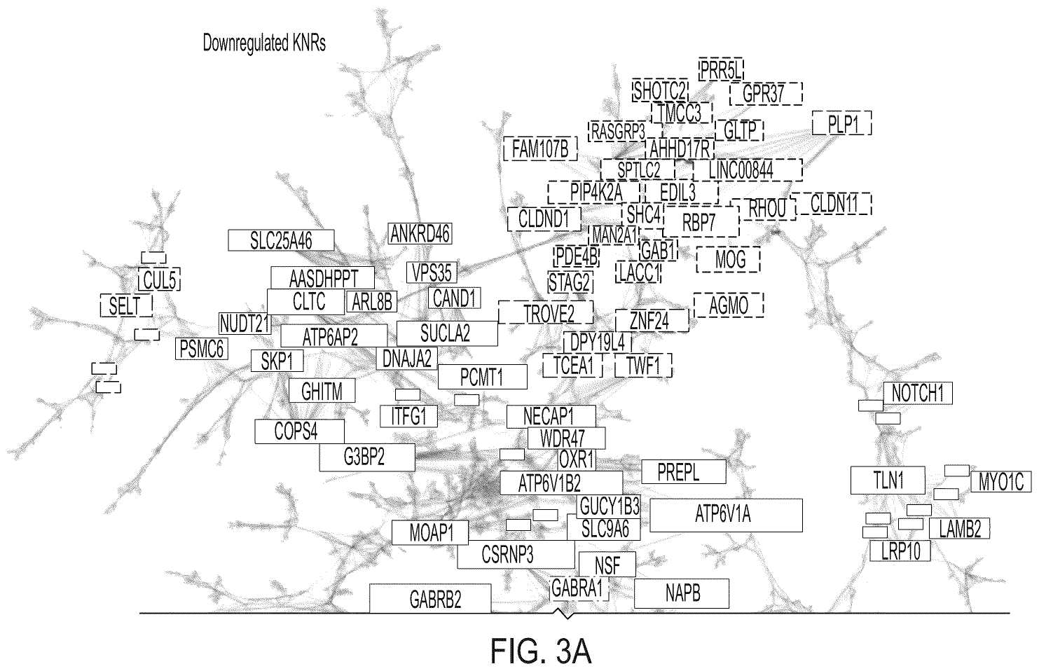

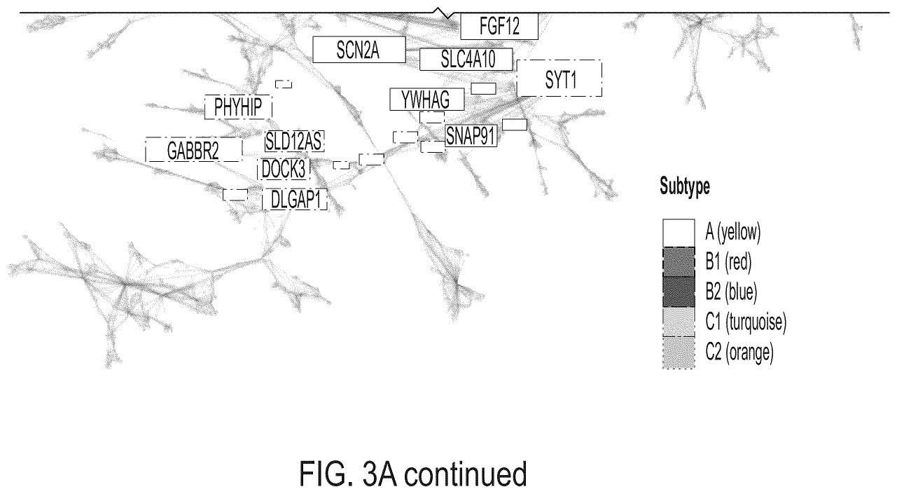

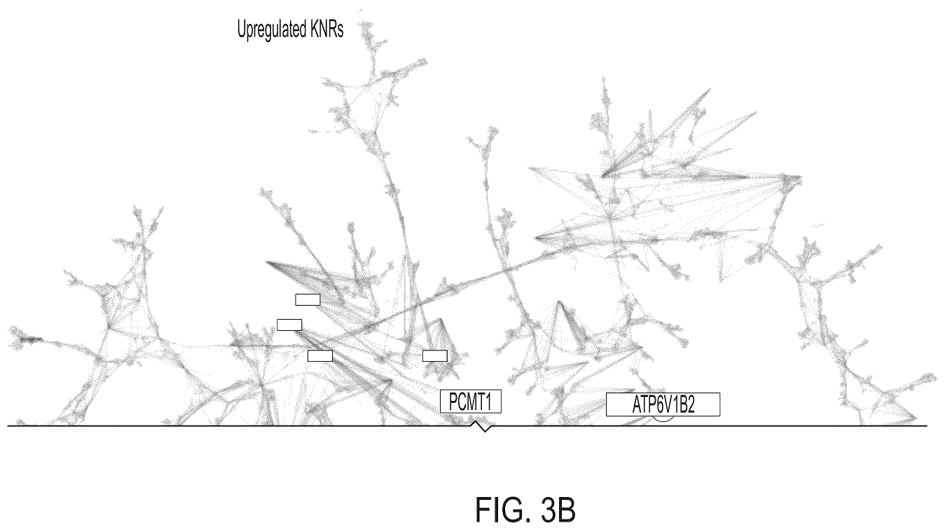

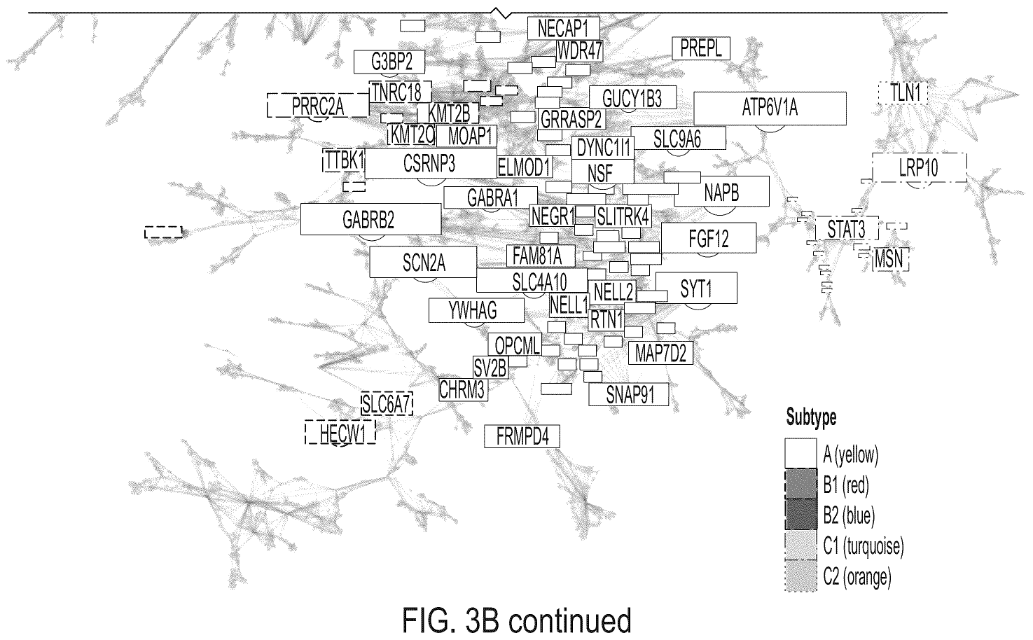

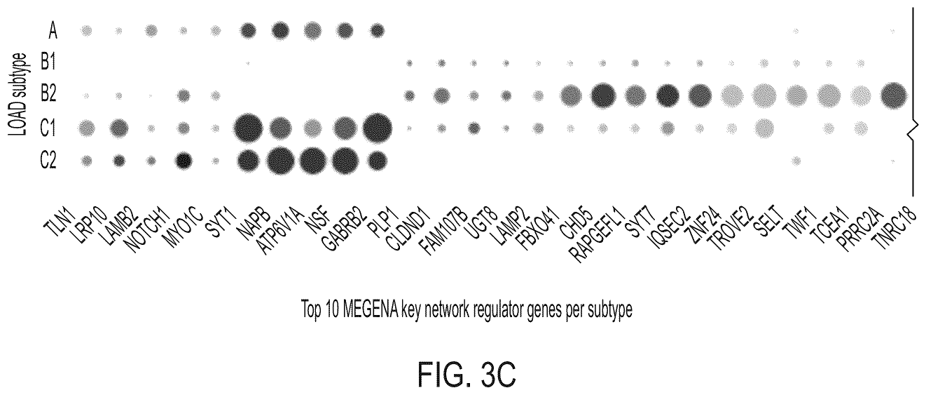

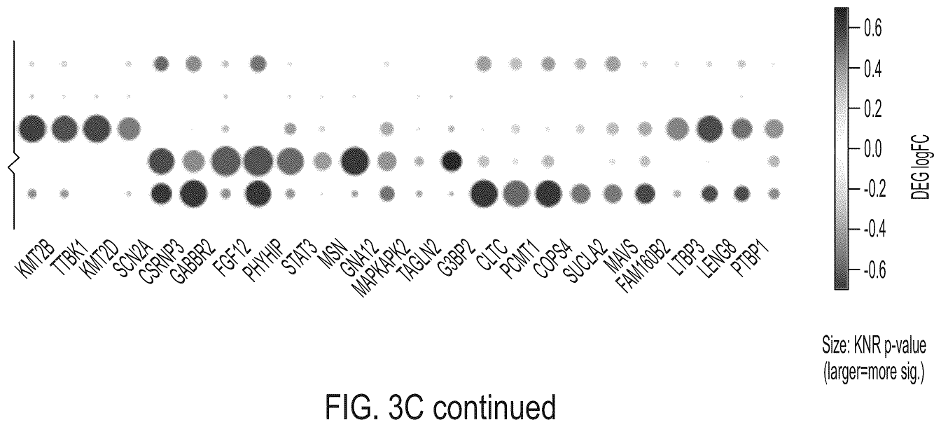

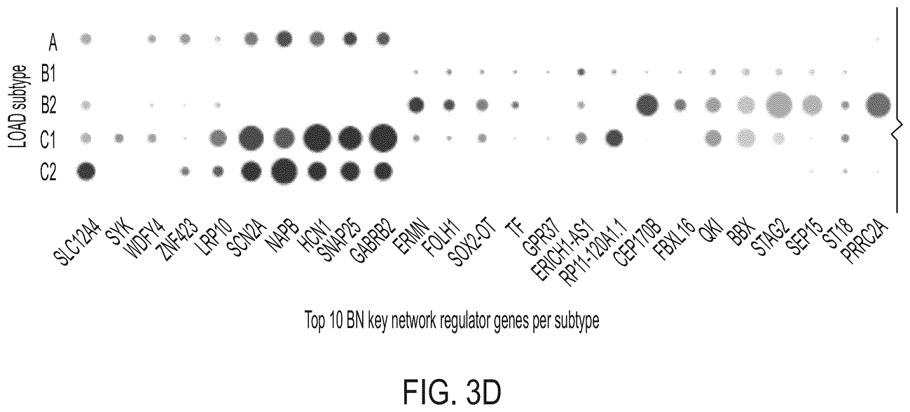

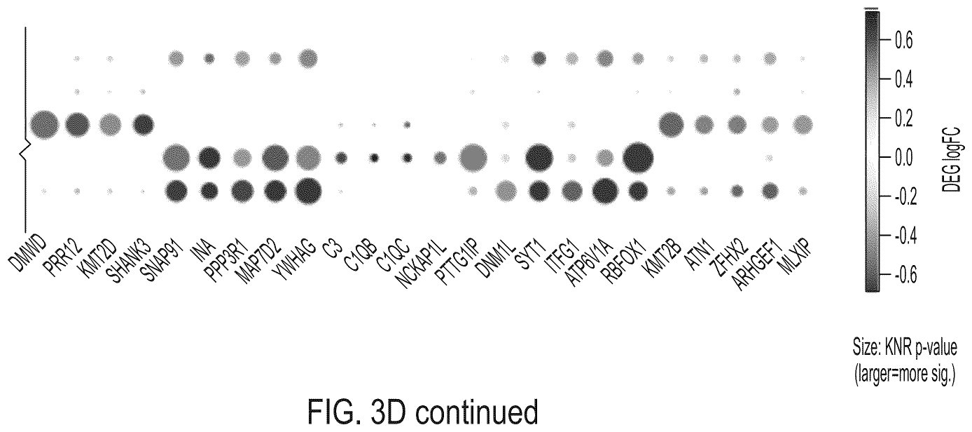

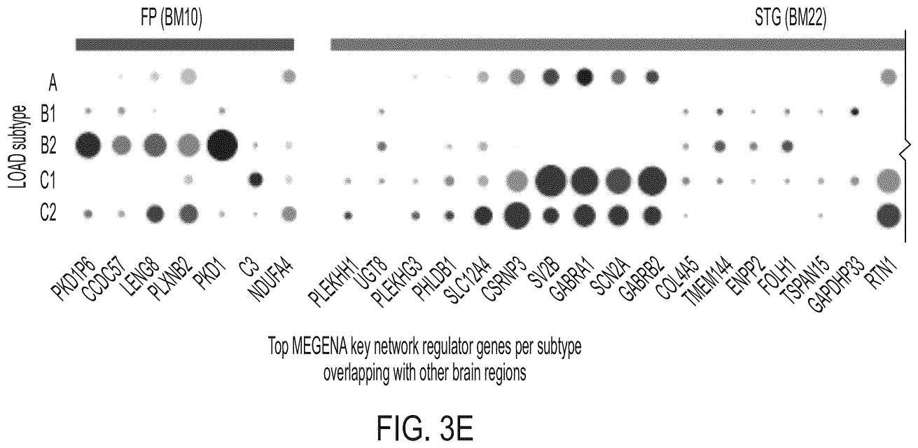

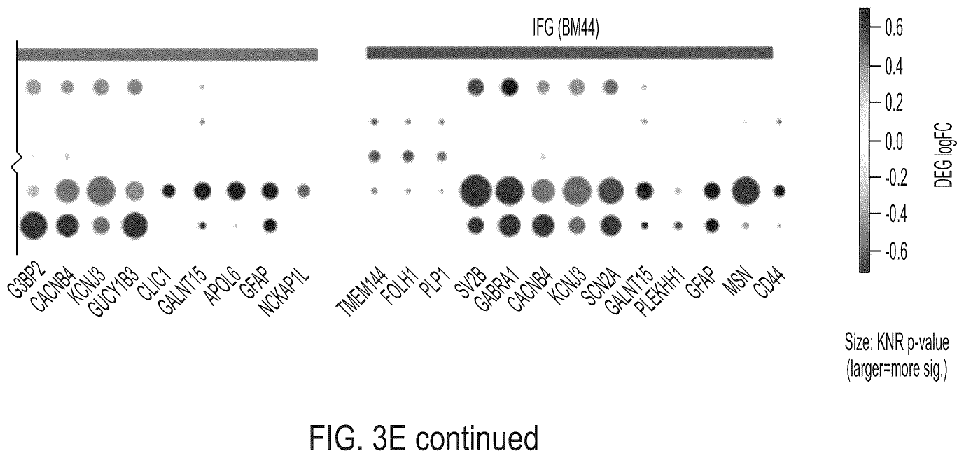

FIG. 3a-b shows the up- and down-regulated key drivers of each subtype in the context of the MEGENA network, while FIG. 3c-d shows the top 20 up- and downregulated KNRs per subtype in the MEGENA and BN networks. Even the subtypes within each class (e.g., B and C) have subtype-specific drivers. As shown in FIG. 3a-b, each subtype's key drivers fall onto separate parts of the MEGENA network, indicating that specific gene modules are subtype-specific, and subtypes are driven by a specific, yet diverse set of disease mechanisms that lead to AD. For instance, the present disclosure shows that numerous neuronal genes located at the center of the global network are downregulated in the amyloid-predominant AD subtypes (C1 and C2) and are predicted to be pathogenic in AD. These downregulated neuronal genes are predicted to be regulated by KNRs GABRB2 (BN p-value: 7.2*10⁻⁴⁴), SYT1 (p-value: 3.6*10⁻³⁰), ATP6V1A (p-value: 1.81*10⁻²⁷), and SCN2A (p-value: 4.07*10⁻¹¹⁶) in both models. On the other hand, the top right of the MEGENA network consists of many oligodendrocytic genes that are downregulated in the class B subtypes and are predicted to be regulated by PLP1 (BN p-value: 1.05*10⁻¹⁴), ERMN (p-value: 1.51*10⁻³²), QKI (p-value: 6.95*10⁻³⁰), and STAG2 (8.89*10⁻²⁷) in both models. Finally, the bottom right of the network is enriched for several downregulated microglial, endothelial and astrocytic genes that are driven by LRP10 (BN p-value: 2.15*10⁻⁸), TLN1 (p-value: 3.71*10⁻⁸), LAMB2 (p-value: 2.2*10⁻⁸), MYO1C (p-value: 5.2*10⁻³), and NOTCH1 (p-value: 4.6*10⁻¹²). Consistent with the inventors' discovery that the AD subtypes in the classes A and C show opposite gene expression changes in known AD-associated gene signatures, many upregulated neuronal KNRs (GABRB2, LRP10, SYT1, and PREPL) in the class A subtype are downregulated in class C. Therefore, the present disclosure shows that both the class A and class C subtypes result from either inhibitory or excitatory dysregulation along a single axis in specific neuronal processes.

Finally, the present disclosure illustrates that these subtype key network regulator genes in the PHG are also key regulators in other brain regions in the MSBB-AD cohort. The same network analysis procedure is applied to the other regions, including the FP, IFG, and STG. As shown in FIG. 3e many neuronal genes, including the KNRs SCN2A, GABRB2, PLP1, and UGT8, and have a consistent direction (up- or down-regulation) in two or more brain regions in the MAPT pathway and amyloid-beta predominant AD subtypes. Furthermore, the inventors found that some key oligodendrocytic genes such as PLP1 and UGT8 remain as key network regulators for the class B subtypes. Therefore, even though the inventors found that the PHG shows the greatest vulnerability, the subtype key regulators have consistent dysregulation in all the regions disclosed here.

### Example 6

### Cell Type Specificity of Subtype Molecular Signatures

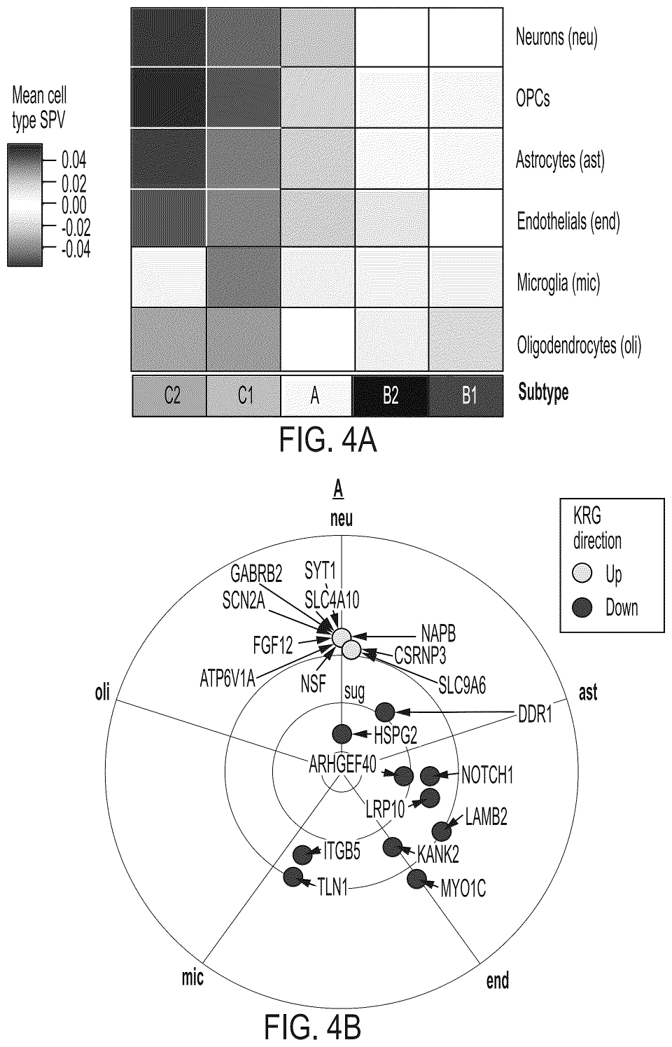

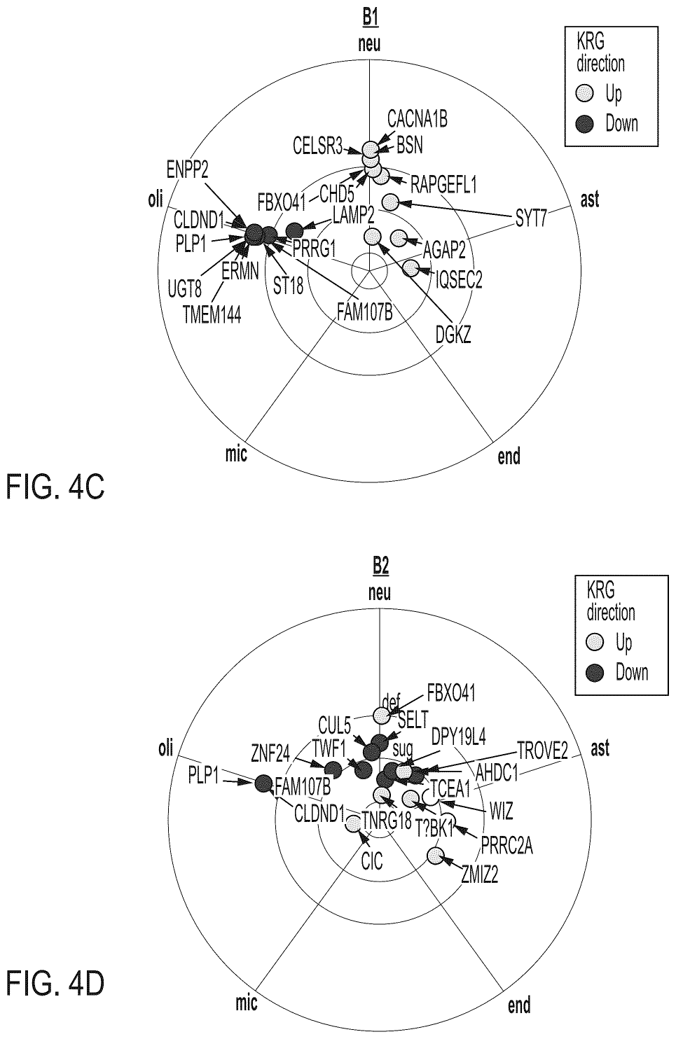

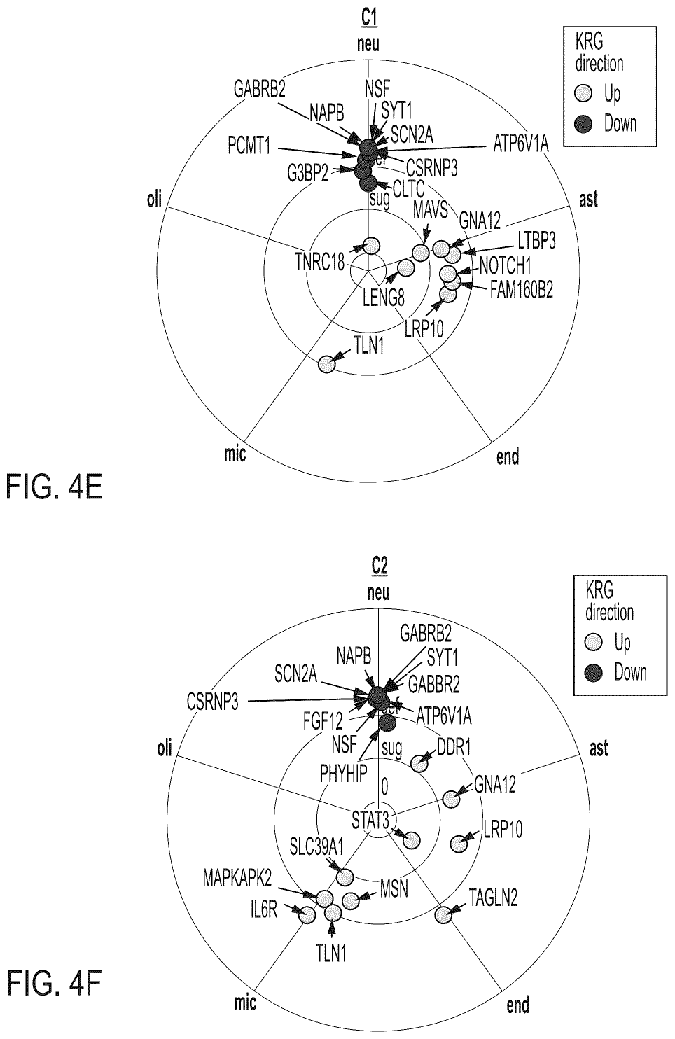

The present disclosure provides a better understanding of brain cell-type specificity of transcriptomic changes in each AD subtype. To achieve this, the inventors performed cell-type proportion analysis on each sample using the brain cell type marker signatures determined by cell-type specific sequencing previously conducted by the inventors (McKenzie et al. 2018). FIG. 4a illustrates significant and unique changes in the cell type composition in the AD subtypes. For instance, class A shows a small increase in neurons combined with a decrease of OPCs, astrocytes, and endothelial cells. On the other hand, class C shows a significant loss of neurons accompanied by an increase of oligodendrocytes, astrocytes, OPCs, and endothelial cells, opposite of the changes observed in class A. The class B subtypes shows mixed changes in other cell types, but both have a small to moderate decrease in oligodendrocytes, consistent with the oligodendrocytic key regulators found through network analysis. The patterns disclosed here are consistent with the reactive astrocytosis and microgliosis commonly seen in some AD patients with immune system activation in response to misfolded or polymerized Aβ(3), which is indicated by the presence of inflammatory markers.

Next, using RNA-seq data derived from cultured brain cells (including neurons, astrocytes, microglia, endothelial cells, and oligodendrocytes), the inventors examined the cell-type specificity of the key regulator genes of each AD subtype. As shown in FIG. 4b-f, the inventors discovered that for the class C (“amyloid-predominant”) subtypes, KDGs are upregulated in microglia (TLN1, MSN, and IL6R), endothelial cells (TAGLN2), and astrocytes (LRP10, GNA12, and LTBP3) and downregulated in neurons (ATP6V1A, SCN2A, GABRB2, and NAPB), consistent with neuroinflammatory destruction. On the other hand, the inventors found that class A subtype has upregulated neuronal regulators (GABRB2, SYT1, and SCN2A) indicating increased neuron remodeling and activity, and downregulated KDGs in astrocytes, endothelial cells, and microglia (LRP10, NOTCH1, MYO1C, and TLN1). They discovered that the class B shows marked downregulation of genes in oligodendrocytes (PLP1, UGT8, CLDND1, ERMN, and ENPP2) with upregulation in other cell types, suggesting that a demyelinating process may be a contributing factor in this class.

### Example 7

Enrichment of Known AD Genetic Risk Markers with Specific Subtypes

To identify the influence of genetic determinants on AD subtype, the inventors investigated the differences in polygenic risk score (PRS) between the predicted AD subtypes. First, each sample's PRS was computed against the Kunkle et al. GWAS meta-analysis across AD using the PRSice R package. As non-European samples are excluded from the meta-analysis, the present disclosure excludes non-European individuals in the MSBB-AD during the PRS calculation. The inventors discovered that two of the three subtype classes show an increase in PRS burden compared with non-demented controls, with significant differences in classes A (median PRS=0.235, p=0.048 under Welsh's t-test) and C (PRS=0.44, p=0.013) subtypes. While the inventors found that class B shows an increase in PRS, this difference is not significant from non-demented controls (PRS=0.33, p=0.117). Additionally, the inventors found an increased PRS burden across all AD samples (median PRS: 0.35) compared with non-demented controls (PRS: −0.43, p=0.016). Despite these significant differences between individual AD subtypes and non-demented controls, there is no significant difference in PRS between the AD subtype classes. Therefore, the present disclosure demonstrates that genetic factors likely predispose individuals in the MSBB cohort to developing AD across each subtype, but such factors fail to adequately discriminate the molecular subtypes.

To better interrogate the intersection between known AD-associated genetic loci and the AD subtypes, the inventors also intersect the AD risk genes compiled by the IGAP Consortium and the predicted subtype-specific key regulators. The inventors found that forty-nine key regulators of the MEGENA network across all five MSBB subtypes have genetic loci associated with AD (IGAP gene-level significance p<=0.01), including AMPH, PICALM, MEF2C, EPDR1, and PSMC6 (Table 3). They also discovered that: AMPH, MEF2C, and EPDR1 are downregulated in the class C AD subtypes and upregulated in class A; PICALM is downregulated in subtype B2; and PSMC6 is downregulated in both the class B and C subtypes.

AMPH, otherwise known as amphiphysin 1, is a vesicle cell surface protein important in clathrin-mediated endocytosis and is primarily expressed in neurons. It is an important homolog of BIN1, which is highly expressed in oligodendrocytes as well as immune cell types and has been shown to be correlated with tau levels as well as GFAP and MBP expression. In neurons, increased AMPH expression is associated with increased tau pathology, while increased BIN1 expression is associated with decreased tau pathology. On the other hand, human autoantibodies to AMPH, which are observed in rare diseases (e.g. Stiff person syndrome), and have been shown to induce defective presynaptic vesicle dynamics and composition, leading to decreased GABAergic transmission. Many GABA pathway genes are also predicted as key regulators in multiple subtypes (GABRB2, GABRA1, GABRA4). Therefore, either up- or downregulation of AMPH may lead to synaptic defects, either through directly on the synaptic vesicles or through the secondary effects of tau accumulation. These data match the present disclosure regarding the class C subtypes showing that decreased AMPH likely lead to decreased neuronal activity and upregulated AMPH likely increase tau pathway activity. Therefore, the effects of GABAergic signaling on cognitive dysfunction are likely critical to understanding and treating AD subtypes.

TABLE 3
Intersection of IGAP Consortium Significant AD Genes (53) and
Predicted AD Subtype Key Network Regulator Genes using MEGENA

| | | | | MEGENA KNR | IGAP gene- | IGAP |
|---|---|---|---|---|---|---|
| | | | | supporting | level | variants |
| Gene | Class | Subtype | Direction | genes | p-value | in gene |
| PICALM | B | blue | down | 48 | 2.7127E−05 | 163 |
| PSMC6 | B | blue | down | 59 | 4.5074E−05 | 5 |
| TRAM1 | B | blue | down | 53 | 0.007827625 | 8 |
| CAMTA2 | B | blue | up | 115 | 0.00008935 | 2 |
| CTIF | B | blue | up | 87 | 0.0083064 | 15 |
| ELL | B | blue | up | 85 | 0.002916457 | 74 |
| FAM193B | B | blue | up | 92 | 0.000886004 | 24 |
| GAK | B | blue | up | 133 | 0.001115133 | 3 |
| HMHA1 | B | blue | up | 50 | 3.44587E−05 | 10 |
| L3MBTL1 | B | blue | up | 74 | 0.004199 | 1 |
| MAML1 | B | blue | up | 108 | 0.000249565 | 31 |
| MARK2 | B | blue | up | 80 | 0.00199503 | 33 |
| PIP5K1C | B | blue | up | 142 | 0.0004533 | 8 |
| RAP1GAP2 | B | blue | up | 67 | 0.00047861 | 3 |
| RASGEF1C | B | blue | up | 71 | 0.008884 | 1 |
| SHANK2 | B | blue | up | 71 | 0.00379017 | 10 |
| VAC14 | B | blue | up | 78 | 0.002427807 | 24 |
| ACP1 | C | orange | down | 87 | 0.001185017 | 6 |
| AMPH | C | orange | down | 81 | 0.0007451 | 1 |
| CHRM3 | C | orange | down | 123 | 0.00029988 | 3 |
| COX7A2L | C | orange | down | 87 | 0.000147388 | 8 |
| EPDR1 | C | orange | down | 77 | 2.1267E−05 | 58 |
| FIG4 | C | orange | down | 71 | 0.000555367 | 3 |
| GUCY1B3 | C | orange | down | 151 | 4.70056E−05 | 5 |
| MEF2C | C | orange | down | 78 | 0.008687003 | 20 |
| PPFIA2 | C | orange | down | 73 | 0.001150956 | 41 |
| PSMC6 | C | orange | down | 152 | 4.5074E−05 | 5 |
| RGS4 | C | orange | down | 74 | 0.0062325 | 6 |
| RTN1 | C | orange | down | 121 | 0.003425 | 2 |
| SLC2A13 | C | orange | down | 85 | 0.00509558 | 5 |
| XRCC5 | C | orange | down | 104 | 0.0015272 | 2 |
| ANTXR1 | C | orange | up | 52 | 0.0021666 | 5 |
| MAML1 | C | orange | up | 110 | 0.000249565 | 31 |
| MAPKAPK2 | C | orange | up | 89 | 0.000764538 | 16 |
| MSI2 | C | orange | up | 67 | 0.0095515 | 2 |
| MTSS1L | C | orange | up | 57 | 0.000141744 | 22 |
| MVB12B | C | orange | up | 81 | 0.001322135 | 26 |
| PARD3B | C | orange | up | 80 | 0.009855 | 2 |
| XKR8 | C | orange | up | 79 | 0.00216636 | 15 |
| PDE4B | B | red | down | 33 | 0.008373333 | 3 |
| PIP4K2A | B | red | down | 42 | 0.008063513 | 40 |
| TRAM1 | B | red | down | 31 | 0.007827625 | 8 |
| CUX2 | B | red | up | 37 | 0.002295725 | 4 |
| PIP5K1C | B | red | up | 36 | 0.0004533 | 8 |
| RAP1GAP2 | B | red | up | 42 | 0.00047861 | 3 |
| RBFOX3 | B | red | up | 34 | 0.0039801 | 10 |
| SHANK2 | B | red | up | 36 | 0.00379017 | 10 |
| AMPH | C | turquoise | down | 81 | 0.0007451 | 1 |
| CADPS | C | turquoise | down | 65 | 0.008799167 | 6 |
| CHRM3 | C | turquoise | down | 129 | 0.00029988 | 3 |
| CSMD1 | C | turquoise | down | 63 | 0.004882021 | 77 |
| CUX2 | C | turquoise | down | 84 | 0.002295725 | 4 |
| EPDR1 | C | turquoise | down | 61 | 2.1267E−05 | 58 |
| GUCY1B3 | C | turquoise | down | 107 | 4.70056E−05 | 5 |
| MEF2C | C | turquoise | down | 80 | 0.008687003 | 20 |
| NGEF | C | turquoise | down | 67 | 0.000173692 | 11 |
| RGS4 | C | turquoise | down | 72 | 0.0062325 | 6 |
| RTN1 | C | turquoise | down | 116 | 0.003425 | 2 |
| SLC2A13 | C | turquoise | down | 81 | 0.00509558 | 5 |
| SYNPR | C | turquoise | down | 63 | 0.001601667 | 3 |
| ANTXR1 | C | turquoise | up | 64 | 0.0021666 | 5 |
| ARHGDIB | C | turquoise | up | 81 | 0.001651 | 1 |
| CMTM7 | C | turquoise | up | 60 | 0.004248222 | 9 |
| DOCK8 | C | turquoise | up | 60 | 0.001083467 | 3 |
| MAPKAPK2 | C | turquoise | up | 157 | 0.000764538 | 16 |
| MSI2 | C | turquoise | up | 51 | 0.0095515 | 2 |
| PARD3B | C | turquoise | up | 91 | 0.009855 | 2 |
| PGF | C | turquoise | up | 81 | 0.001583667 | 3 |
| TRAM1 | C | turquoise | up | 61 | 0.007827625 | 8 |
| ANTXR1 | A | yellow | down | 36 | 0.0021666 | 5 |
| HMHA1 | A | yellow | down | 32 | 3.44587E−05 | 10 |
| PARD3B | A | yellow | down | 48 | 0.009855 | 2 |
| XKR8 | A | yellow | down | 51 | 0.00216636 | 15 |
| AMPH | A | yellow | up | 62 | 0.0007451 | 1 |
| CHRM3 | A | yellow | up | 93 | 0.00029988 | 3 |
| EPDR1 | A | yellow | up | 63 | 2.1267E−05 | 58 |
| GUCY1B3 | A | yellow | up | 111 | 4.70056E−05 | 5 |
| MEF2C | A | yellow | up | 66 | 0.008687003 | 20 |
| RGS4 | A | yellow | up | 62 | 0.0062325 | 6 |
| RTN1 | A | yellow | up | 102 | 0.003425 | 2 |
| SLC2A13 | A | yellow | up | 75 | 0.00509558 | 5 |

### Example 8

Sub-Classification of MSBB-AD Samples with Mild Cognitive Impairment (CDR=0.5) Recapitulates Three Major AD Subtype Classes

The present disclosure shows that patients with a mild cognitive impairment (MCI), without clinically defined AD, exhibit a subtype-specific signature consistent with the AD subtypes identified here. Based on the clinical data available, the inventors defined MCI for the MSBB-AD cohort as having a CDR score of 0.5, which corresponded to possible or mild dementia (n=32). To differentiate whether MCI samples group together with AD subtypes or if MCI samples cluster separately based on their molecular signatures, the inventors repeated the WSCNA on the combined AD and MCI samples in the MSBB-AD cohort. The inventors discovered that MCI samples are distributed in the branches corresponding with all five AD subtypes, including both amyloid-beta and tau-predominant AD subtypes. A count of the number of MCI samples distributed to each branch, labeled by the corresponding AD subtype is shown in Table 4. The inventors found that the distribution of the MCI samples across the subtypes is different than that of the AD samples (one-way ANOVA p-value: 4.7*10⁻³, F-statistic=14.947, df=1). The MCI samples are distributed more often to tau-predominant AD subtypes than amyloid-beta predominant AD subtypes. This could be due to a variety of reasons, including potential resilience to certain AD subtypes among the MCI group. Therefore, the present disclosure provides evidence that MCI samples may also be sub-classified into different subtypes, providing additional insights into molecular features of the disease.

TABLE 4
Sub-classification of MCI samples in MSBB-AD

| | MCI | Percent of | AD | Percent of |
|---|---|---|---|---|
| Subtype | samples | MCI samp. | samples | AD samp. |
| C1 | 1 | 3.1% | 34 | 18.6% |
| C2 | 1 | 3.1% | 15 | 8.2% |
| B1 | 11 | 34.4% | 39 | 21.3% |
| B2 | 5 | 15.6% | 34 | 18.6% |
| A | 14 | 43.8% | 61 | 33.3% |

### Example 9

### MSBB-AD Subtypes in ROSMAP Show Conserved AD Subtype Molecular Signatures

WSCNA-based subtyping analysis on the gene expression data from the dorsolateral prefrontal cortex (DLPFC) in the ROSMAP cohort (n=610, with 388 AD cases) confirms the AD subtypes identified in the MSBB-AD cohort. The ROSMAP cohort is an independent study of a different brain region with more mild cognitive impairment (n=287) (MCI) and severe AD patients. The post-mortem brains in the ROSMAP cohort were collected from individuals residing around multiple geographically distant sites across the United States, and patients enrolled in the study were evaluated multiple times over many years before death for cognitive impairment that was suggestive of AD. Cognitive impairment was measured by the mini-mental status exam (MMSE) at multiple timepoints, and pathologic factors such as amyloid-beta and tau burden were measured post-mortem. Additionally, the study includes three predominantly African American and Hispanic communities in multiple locations. Therefore, re-identification of similar molecular subtypes of AD in the ROSMAP cohort should allow for greater generalization of the findings from the MSBB-AD alone.

As with the normalization process performed on the MSBB RNA-seq data, the inventors corrected the ROSMAP gene expression data for batch effect, post-mortem interval, gender, RNA integrity number, and outliers, as well as dementia severity using MMSE scores to remove any potential effect of increasing neuronal loss with AD staging on the ROSMAP subtypes. Previous studies have shown a strong correspondence between CDR scores and MMSE (Bennet et al. 2012), and, therefore, MMSE would serve as a similar measure of dementia severity. The inventors measured the cell type proportion of ROSMAP samples with increasing dementia severity before and after MMSE normalization. Using these methods, the inventors discovered that—like in MSBB-AD—neuronal loss inferred from bulk transcriptomic data in ROSMAP is correlated with decreasing cognition (MMSE), and normalization using MMSE score can eliminate this observed bias and allows for stage-free subtyping. Furthermore, the inventors show in the present disclosure that cell type proportion normalization is not sufficient to eliminate the bias from dementia severity, as a significant residual correlation still persists between MMSE and various cell types. Therefore, the inventors used MMSE normalization to correct for this.

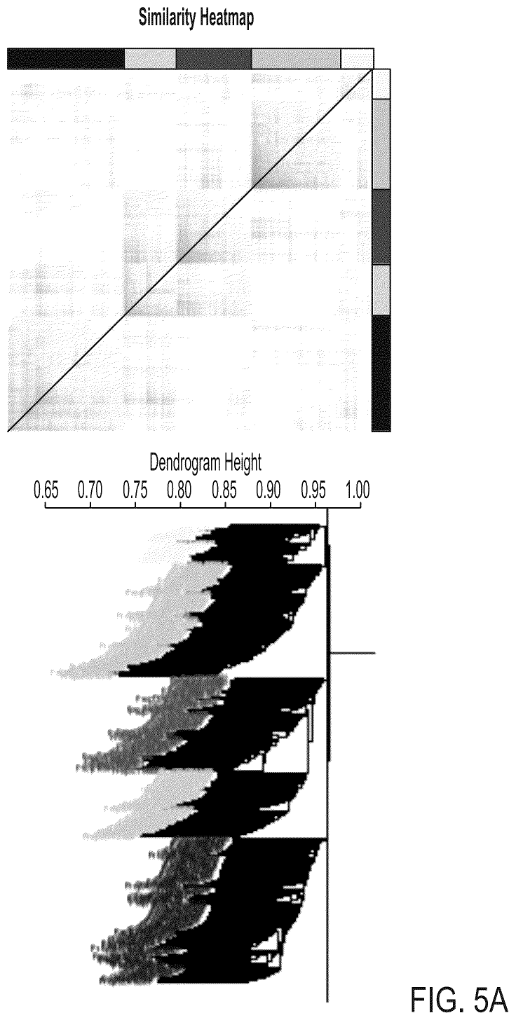

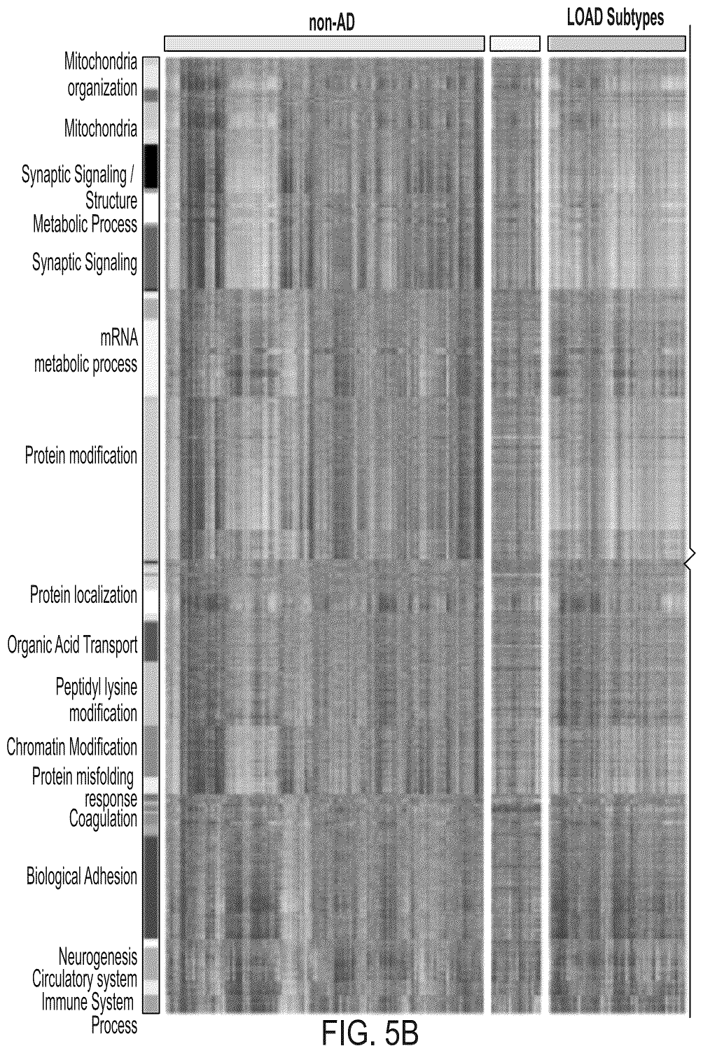

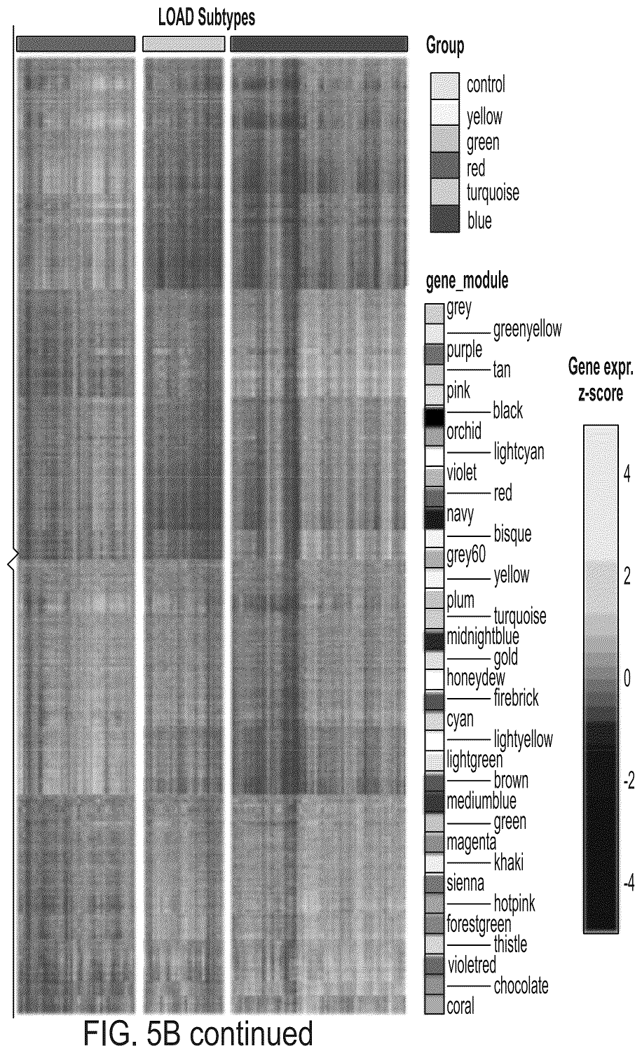

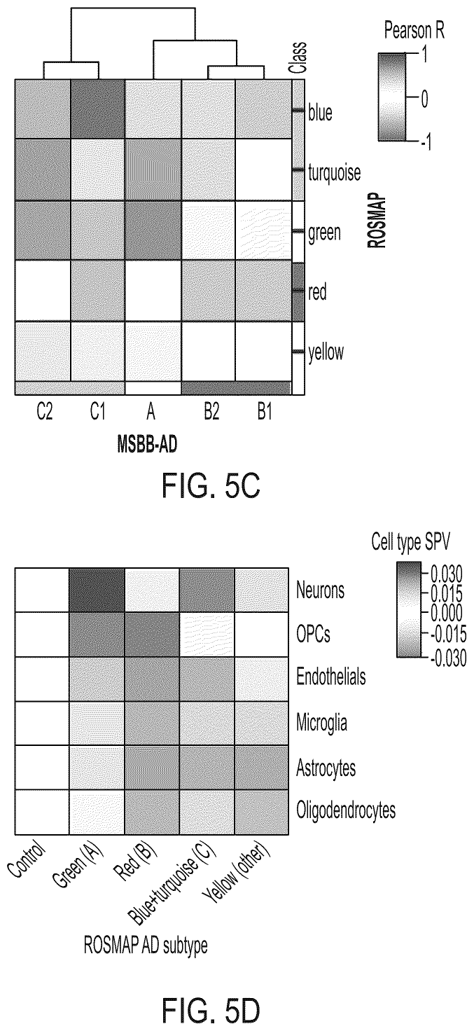

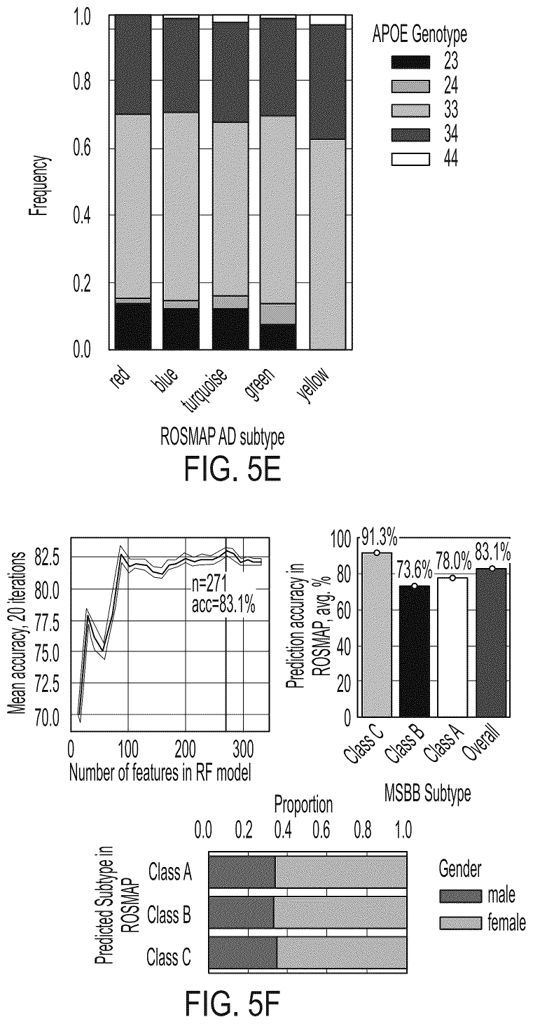

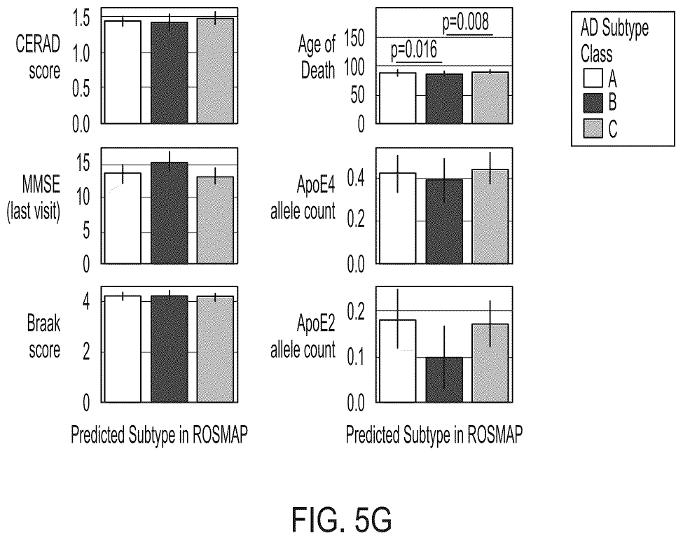

As shown in FIG. 5a, the inventors identify five subtypes in this cohort, similar to those in the MSBB-AD cohort. Expression profiles from this cohort and from the subtypes in the PHG of the MSBB-AD cohort are significantly correlated (FIG. 5b-c). First, by inspection of the up and downregulated gene modules alone, the inventors reveal two subtypes with decreased synaptic signaling and increased immune response, one subtype with increased synaptic signaling and protein modification activity, and two subtypes that do not show changes in either pathway. Next, to quantify the AD similarity between the two sets of AD subtypes in the two independent cohorts, the inventors performed correlation analysis of the mean gene expression profiles of these subtypes, followed by hierarchical clustering analysis. As shown in FIG. 5c, most subtypes in the ROSMAP cohort match well certain subtypes from the PHG in the MSBB-AD cohort, except the yellow subtype in the ROSMAP (Pearson correlation coefficients between 0.6-0.8). The inventors' clustering analysis further reveals that three major AD subtype classes (tau-predominant classes A and B, and amyloid-beta predominant class C) are highly conserved across both cohorts with different brain regions.

The present disclosure also shows that the subjects in the ROSMAP cohort are distributed in the AD subtypes at the same relative proportion as those in the MSBB-AD cohort. Surprisingly, the distributions are roughly proportional to each other, with fewer samples in the C2 subtype than C1, with most samples either falling into class A or B (Table 5). The inventors found no significant difference in the distribution of samples after performing a one-way ANOVA test across both MSBB-AD and ROSMAP, while and excluding samples that don't match any subtype across both datasets (p-value: 0.249, f-statistic=1.62, df=1).

TABLE 5
Relative sample proportion per AD subtype across
MSBB-AD and ROSMAP

| | MSBB-AD | Percent of | ROSMAP | Percent of |
|---|---|---|---|---|
| Subtype | samples | MSBB-AD | samples | ROSMAP |
| C1 | 34 | 18.6% | 83 | 28.9% |
| C2 | 15 | 8.2% | 43 | 15.0% |
| B1 + B2 | 73 | 39.9% | 63 | 22.0% |
| A | 61 | 33.3% | 74 | 25.8% |
| Others (unknown) | 0 | 0% | 24 | 8.4% |

As shown in FIG. 5d, the inventors found that the ROSMAP subtypes also exhibit predicted cell-type proportion changes in the frontal cortex. For example, the class C-like subtypes show decreases in neuronal SPVs and increases in microglia and endothelial cell SPVs. On the other hand, inventors found that the class A-like subtype shows strong increases in neuronal and OPC SPVs, along with decreases in other cell types. Finally, the inventors' analysis reveals that the class B-like subtype shows strong decreases in OPC SPV only. Therefore, the present disclosure reveals that AD subtypes show strong, characteristic susceptibility to cell type proportion changes that are consistent across the subtypes identified in the two independent cohorts.

The present disclosure shows that, similar to the MSBB subtypes, the ROSMAP subtypes are not associated with clinical and pathological traits including biological sex, cognitive scores, age, CERAD pathologic scores, MMSE, and Braak staging, revealing that these subtypes are truly molecular subtypes with distinct expression patterns. As shown in FIG. 5e, APOE e2 allele dosage significantly decreases in the yellow (“other AD”) subtype compared to all other subtypes (p-values<0.025 under the Welsh's t-test), which did not contain any APOE e2 individuals. On the other hand, the present disclosure shows that APOE e4 allele dosage is not significantly associated with ROSMAP AD subtypes.

Additionally, the inventors computed the polygenic risk scores for ROSMAP subtypes and non-demented controls using the Kunkle et al. GWAS meta-analysis, applying the same methodology as for MSBB-AD. Similar to the MSBB-AD cohort, the present disclosure shows that each of the ROSMAP classes A (median: 0.339, p-value=5.1*10⁻⁴ under Welsh's t-test), B (med.: 0.084, p=0.032), and C (med.: 0.191, p=1*10⁻⁵) have an increased PRS compared to non-demented controls (med.: −0.239); however, the inventors found no significant PRS differences between subtypes.

### Example 10

Correspondence Between huma AD Subtypes and AD Mouse Models

In the past two decades, several different mouse models of AD have been developed to characterize AD pathology, biology, and behavioral changes. Many of these mouse models perturb various AD-related proteins or regulatory genes such as AD, APP, Tau, PSEN1, TYROBP, and HDAC1. Given the significant differences in molecular changes among the AD subtypes, the inventors examined whether these AD subtypes' transcriptomic signatures match the existing mouse model signatures.

The inventors collected aligned RNA-seq data from 19 mouse model studies of AD that are publicly available at the Accelerating Medicines Project-Alzheimer's Disease (AMP-AD) portal on Synapse.org and GEO. Many of these models harbor multiple amyloid precursor protein (APP) variants and/or Tau protein variants. The Swedish mice (APPK670NM671NL) develop amyloid plaques near neurons. The Dutch (APPE693Q) mice accumulate soluble Aβ in perivascular cells at the blood-brain barrier. The 5XFAD mice recapitulate APP variants seen in familial forms of AD but do not have related tau NFT seen in AD. Tau protein variant TauP301L (‘D35’) and TauP301S (‘PS19’) mice develop hyperphosphorylated tau, as well as presenilin 1 (PSEN1) Δexon9 and M146V variants. The inventors also examined APOE variant mice as well as mouse models with mutant HDAC1, TYROBP, TREM2, BIN1, CD2AP, CL U, and GFAP alleles.

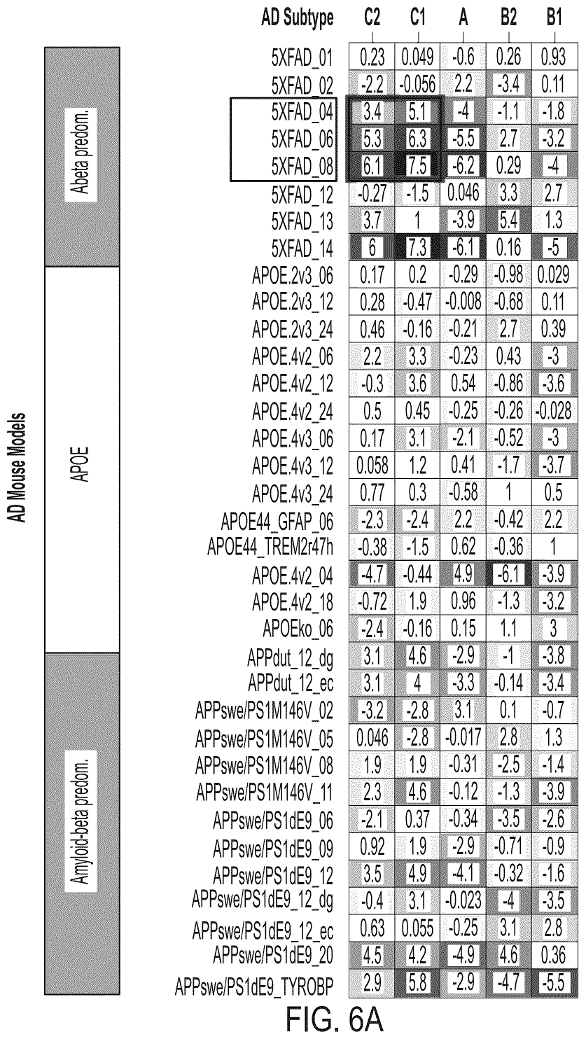

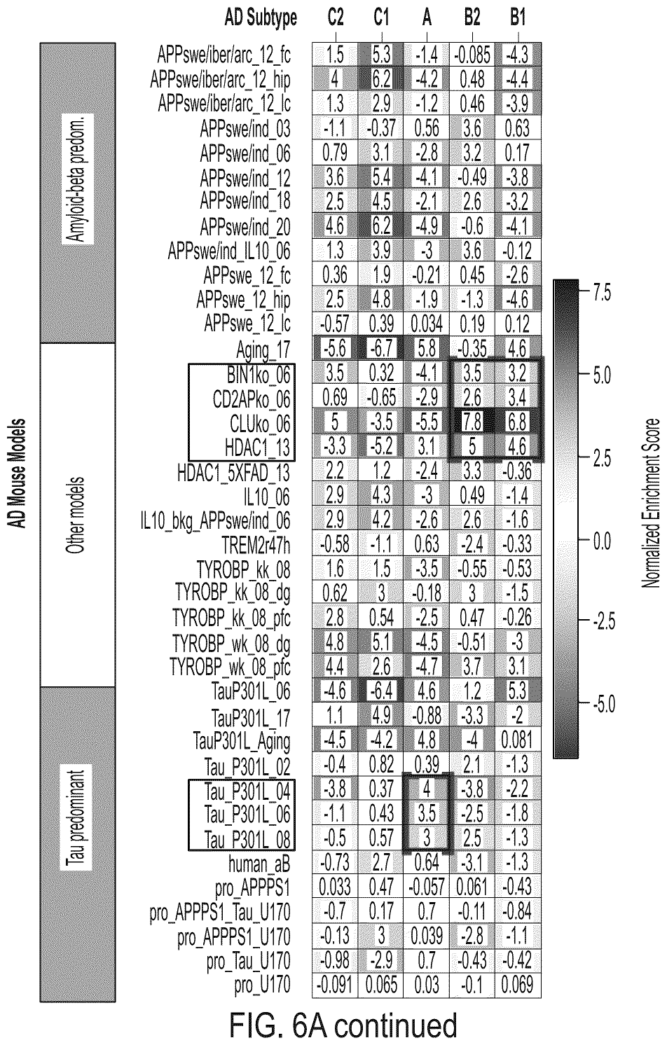

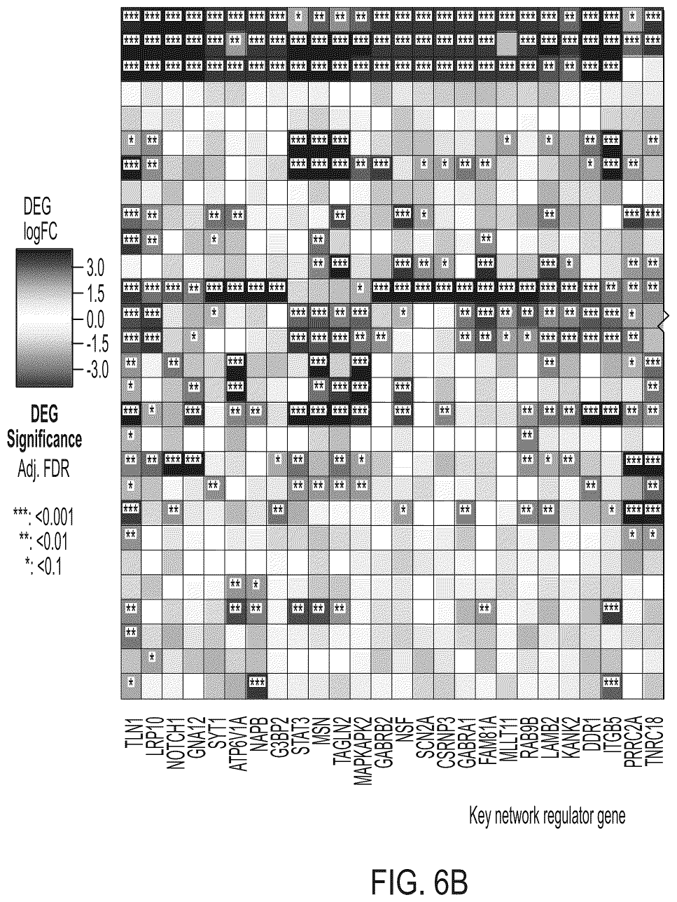

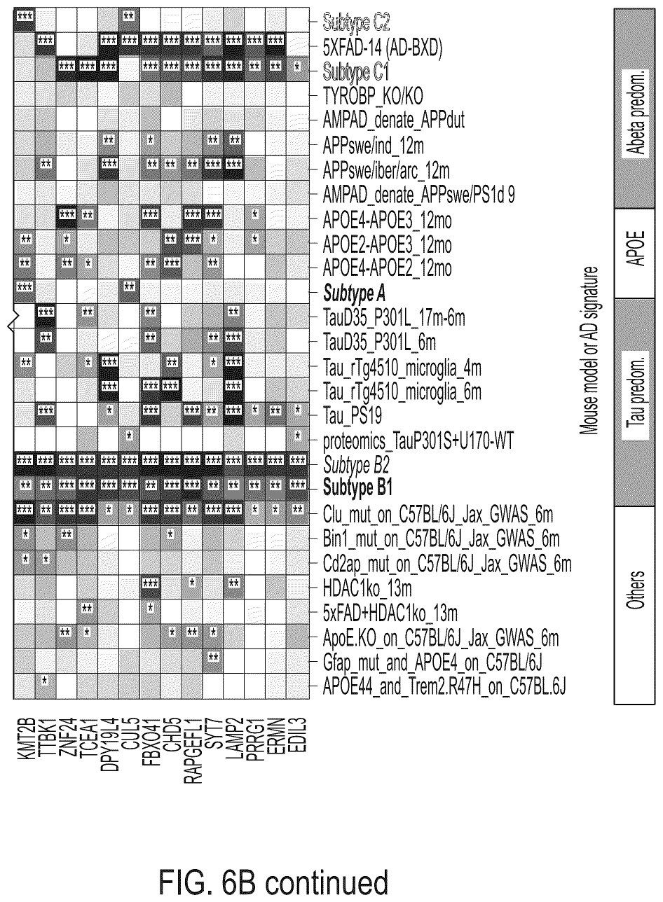

As shown in FIG. 6a, the inventors found that the class C subtypes (amyloid-beta predominant) match to the 5XFAD (familial), APP Dutch (inflammatory), and APP Swedish (amyloid) mice, consistent with an amyloid-driven disease with increased immune and circulatory system activity, as well as previous findings that the class C subtypes may be driven by inflammatory processes (shown in FIG. 1e). Inflammation at the blood-brain barrier has been noted in AD as well as other age-related neurodegenerative diseases such as vascular dementia. On the other hand, the inventors found that the tau-predominant class A subtype has a gene signature opposite to those of 5XFAD and APP mouse models, but consistent with that of the TauP301L model and this is in line with gene expression changes in known tau pathways. Furthermore, the inventors found that the remaining two class B subtypes (B1 and B2) show the strongest match with the CLU (apoJ-) mutant model and a good match with the CD2AP and BIN1 mutant models. Clusterin, a secreted neuroprotective glycoprotein secreted primarily by astrocytes, has been shown to be increased in AD in response to tau-mediated neurodegeneration, and the Clusterin mutant mice have been shown to have less Aβ damage and neuritic dystrophy when bred with 5XFAD model mice versus controls. Therefore, the present disclosure shows that the class B subtypes match the mouse models that carry tau-related neurodegenerative factors over amyloid-related factors.

The inventors further examined the expression changes of the subtype-specific key regulators in the 5XFAD, TauP301L, and CLU mutant mouse models that match the three subtype classes. FIG. 6b shows the gene expression levels of the top four key regulators from each of the five MSBB-AD subtypes in each mouse model. The present disclosure reveals that the gene expression differences between the AD subtypes across human subjects are recapitulated in specific mouse models. The inventors show here that many KNRs of the amyloid-beta and tau-predominant subtypes have consistent expression changes in the respective human brain samples and the matched mouse models.

### Example 10

### Predicting AD Subtypes Using Whole Genome Sequencing Data

Three major Alzheimer's Disease (AD) subtypes (i.e., typical, intermediate, and atypical subtypes) were identified using the gene expression data in multiple brain regions in the MSBB and ROSMAP cohorts using whole genome sequencing (WGS) data. Copy number variations (CNVs) were identified from the paired-end short read (2×150 bp)-based whole-genome sequencing (WGS) data generated from postmortem brain tissues of 1,411 North American Caucasian individuals across two cohorts from the Accelerating Medicines Partnership-Alzheimer's Disease (AMP-AD) consortium, including the Mount Sinai/JJ Peters VA Medical Center Brain Bank (MSBB) AD cohort, and the ROSMAP cohort using four complementary CNV calling approaches (i.e., CNVnator, Pindel, MetaSV, and Delly2). Within each cohort, individual-level calling results from the four approaches were integrated into a set of population-level CNVs. Furthermore, only consensus CNVs detected by three or more approaches in each cohort were used for afterward analysis to exclude software bias.

By comparing 701 LOAD cases with 710 non-AD cases, the inventors identified 3,012 rare AD-specific CNVs genome-wide. The inventors discovered that AD-specific CNVs were only observed in AD cases and found that sixty-four AD-specific CNVs were conserved across two cohorts. The inventors further found that AD-specific CNVs are enriched in transcriptional regions for biological processes such as cellular glucuronidation, neuron projection, and multicellular organismal signaling, a novel finding not found in AD GWAS. By further integrating clinical, pathophysiological, and transcriptomic data, the inventors discovered that common CNVs affect the transcription levels of genes involved in MHC Class II receptor activity across different brain regions, supporting previous reports of the increased immune response in AD. The inventors discovered that three CNVs (i.e., mCNV233, mCNV236, and mCNV11665) are significantly negatively correlated with the Braak score in the DLPFC region. The CNV-Gene-Trait correlation networks integrating matched multi-omics and clinicopathological data disclosed here first pinpoint one novel CNV, a key regulator for immune response (DEL6619.MSBB/mCNV21544.ROSMAP), and further provide many novel gene targets which connect CNVs and clinical and pathological traits of AD.

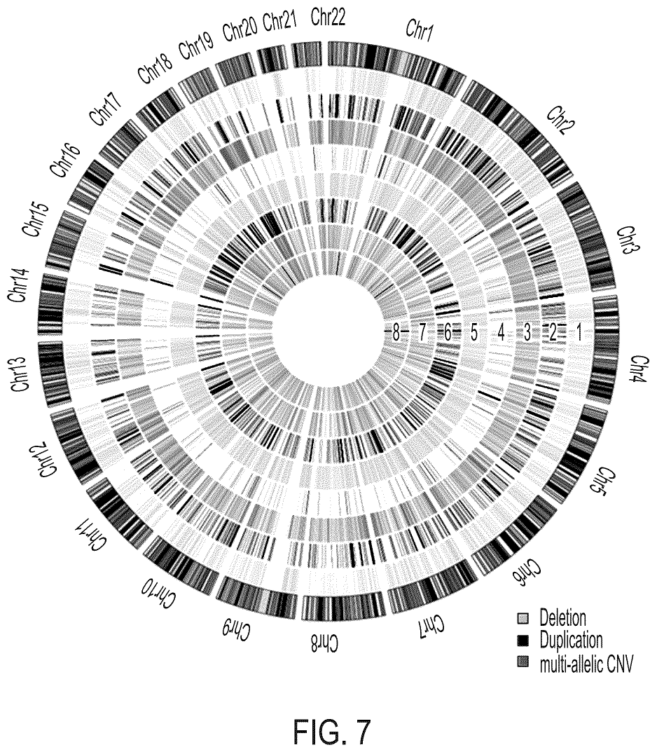

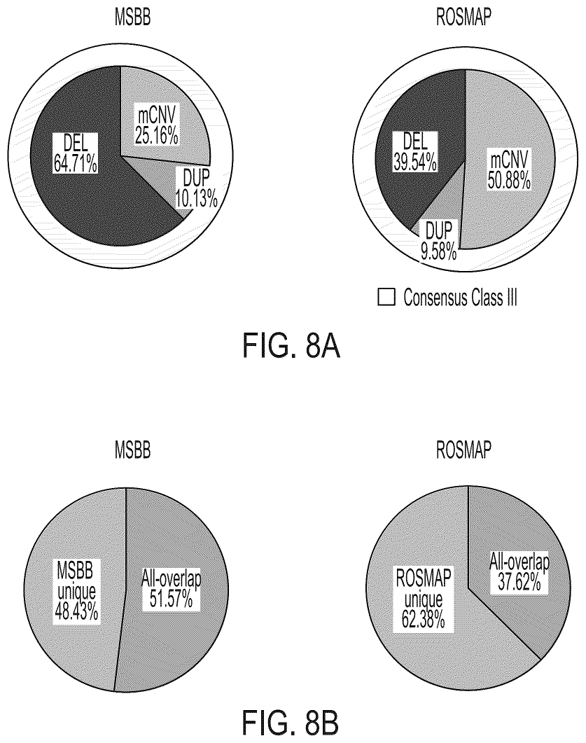

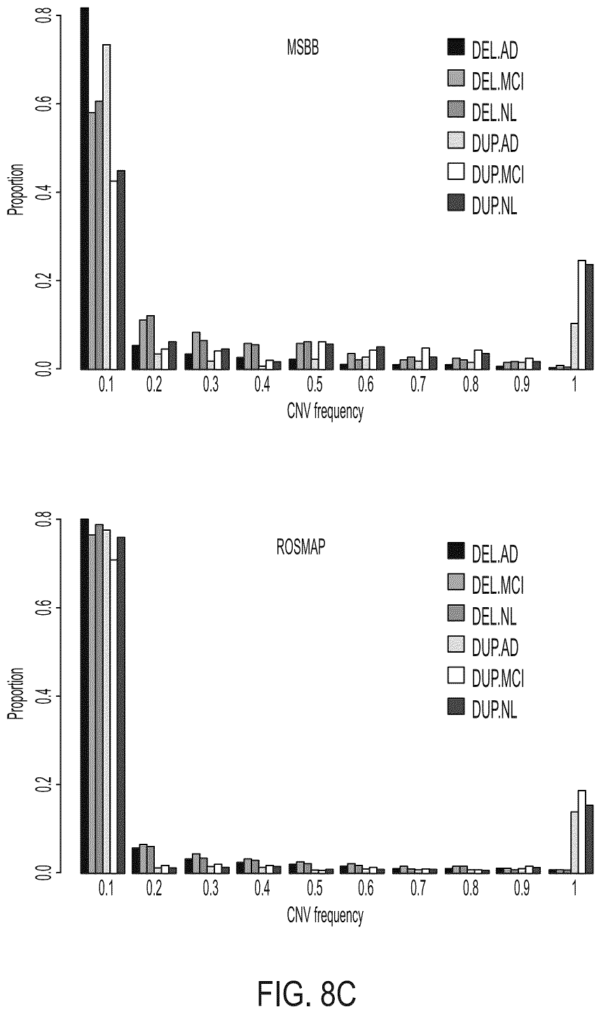

After excluding the duplications, contaminated samples, and outliers, the MSBB and ROSMAP cohorts contain 341 and 1,129 samples, respectively. To exclude bias from demographic history, the analysis focused on North American Caucasian samples in the afterward analysis. There were 1,411 samples left in total (MSBB: 284 samples, and ROSMAP: 1,127 samples). By integrating results from four different and complementary CNV calling approaches (CNVnator, Pindel, Delly2, and MetaSV), a set of CNVs was generated for each cohort (FIG. 7 and FIG. 8, Table 6). The robustness of these CNVs was further evaluated by the consensus among the four CNV calling approaches. Consensus Class I includes the CNVs identified by only one calling method, and Consensus Class II consists of the CNVs determined by only two methods, while Consensus Class III contains the CNVs identified by three or more methods. The analysis of the present disclosure focused on the CNVs in the Consensus Class III in the subsequent analyses to exclude method bias.

The Consensus Class III includes 7,150 and 9,902 CNVs in the MSBB and ROSMAP cohorts, respectively (Table 6, FIG. 8A). Two CNVs with a reciprocal overlap (RO) of 50% or greater in their genomic locations are considered to have significant overlap and are treated as the same CNV. The median individual CNV counts of the two cohorts are similar (i.e., 987 CNVs per individual in the MSBB, and 1,052 in the ROSMAP cohort). The two cohorts share 3,687 CNVs based on the RO threshold of 50% (FIG. 8B). To estimate the CNV calling pipeline's replication rate, four samples were picked at random (i.e., three AD cases and one NL control) from the MSBB cohort, the corresponding genomes were sequenced twice, and the CNV calling results from two batches were compared. The inventors discovered that their CNV calling pipeline's replication rate ranged from 97.30% to 98.63%. A further comparison of the consensus CNV sets was made with four public CNV datasets based on large populations (i.e., Decipher, DGV, the 1000 Genome project, and GnomAD). More than half of the CNVs were validated in the four public CNV datasets. The inventors discovered that the overlaps between the present consensus CNV sets and these public CNV datasets were generally greater than the overlaps between the public datasets. For example, they discovered that the overlaps of the MSBB, ROSMAP CNV datasets and the GnomAD CNV dataset were approximately 74% and 59%, respectively, whereas the overlaps between GnomAD and DECIPHER, 1KGP, and DGV were approximately 39%, 51%, and 31%, respectively.

TABLE 6
Summary of detected consensus autosomal CNVs
from MSBB and ROSMAP

| CNV type | Calling Quality | MSBB | ROSMAP |
|---|---|---|---|
| Bi-allelic | Consensus Class III | 4,627 | 3,915 |
| deletions | | | |
| Bi-allelic | Consensus Class III | 724 | 949 |
| duplications | | | |
| Multi-allelic | Consensus Class III | 1,799 | 5,038 |
| CNVs | | | |
| Total CNVs | | 7,150 | 9,902 |

Note:
Consensus class is defined by the supported software number. The consensus class III represents CNVs detected by three or more software.

The inventors further categorized all samples of the MSBB and ROSMAP cohorts into three clinical diagnostic groups (i.e., the AD group, the mild cognitive impairment (MCI) group, and the normal control (NL) group) based on the disease severity measurement Clinical Dementia Rating (CDR). In the MSBB cohort, there are 224 AD samples with CDR>0.5, 27 MCI samples with CDR=0.5, and 33 NL samples without cognitive impairment (CDR=0). The ROSMAP cohort includes 477 AD samples, 285 MCI samples, and 365 NL samples. In total, there are 701 LOAD, 312 MCI, and 398 NL samples.

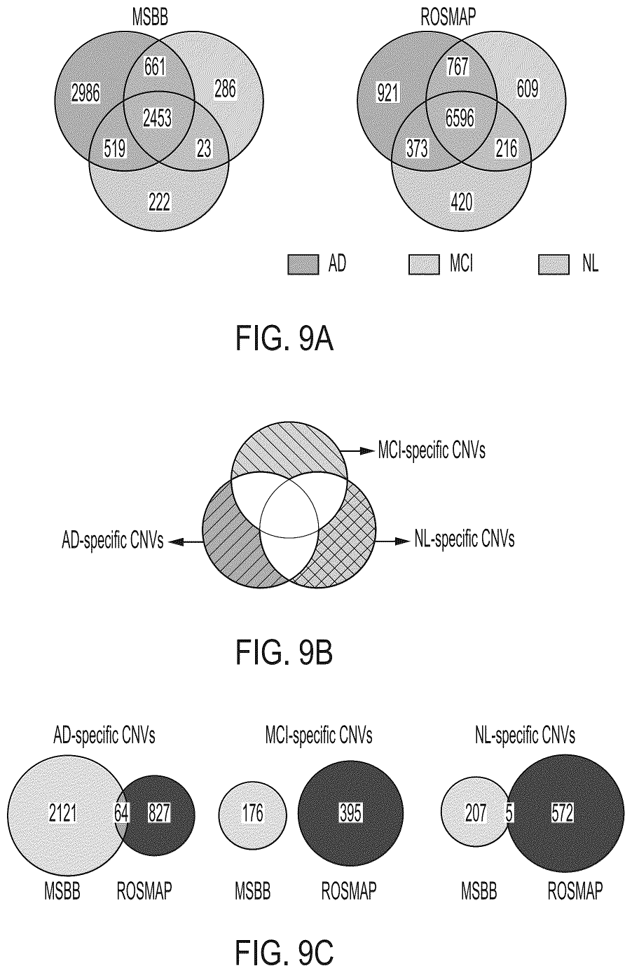

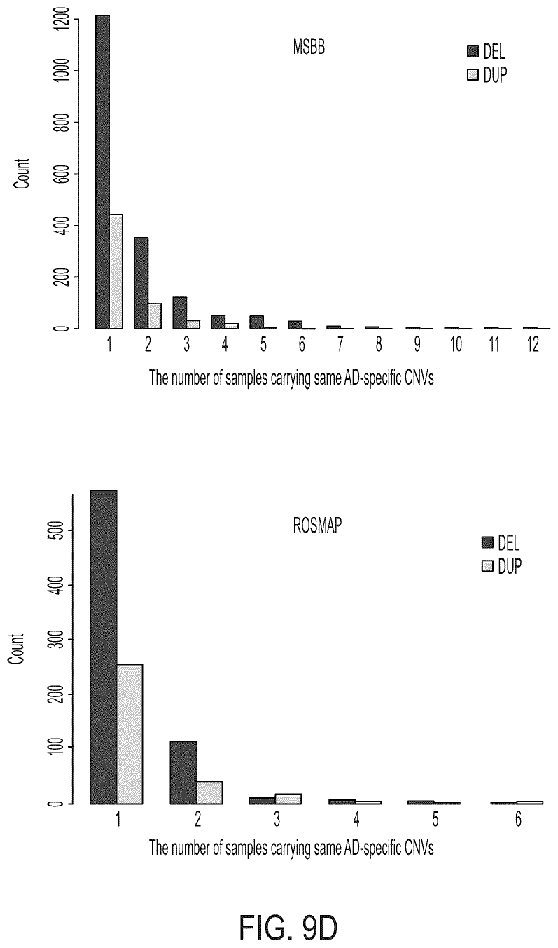

Each CNV was assigned to a clinical diagnostic group to which the respective sample belonged (FIG. 9A). Group-specific CNVs are defined as CNVs that are only observed in one specific group but not in any other group (FIG. 9B). For example, the AD-specific CNVs are CNVs only observed in the AD cases in the two cohorts under study but not in the NL and MCI cases. If the frequency of a CNV in the AD group is greater than 0 and its frequency in the non-AD groups (i.e., the MCI and NL groups) is zero, this CNV is called an AD-specific CNV. Similarly, the MCI-specific CNVs are only observed in the MCI cases, while the NL-specific CNVs are only observed in the NL cases (FIG. 9B). By excluding the CNVs detected in any of the 710 non-AD cases (i.e., the 312 MCI cases and 398 NL), the inventors identified 3,012 unique AD-specific CNVs in the 701 AD cases from the MSBB and ROSMAP cohorts (MSBB: 2,185, ROSMAP: 891) (FIG. 9C).

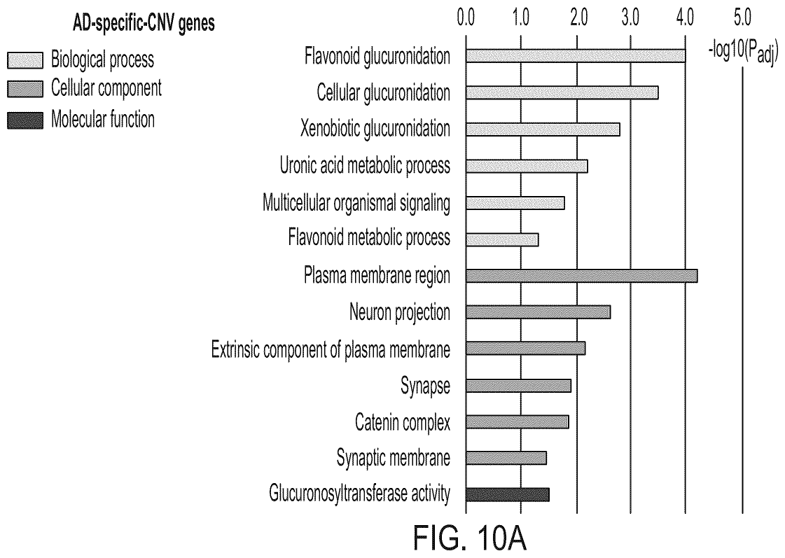

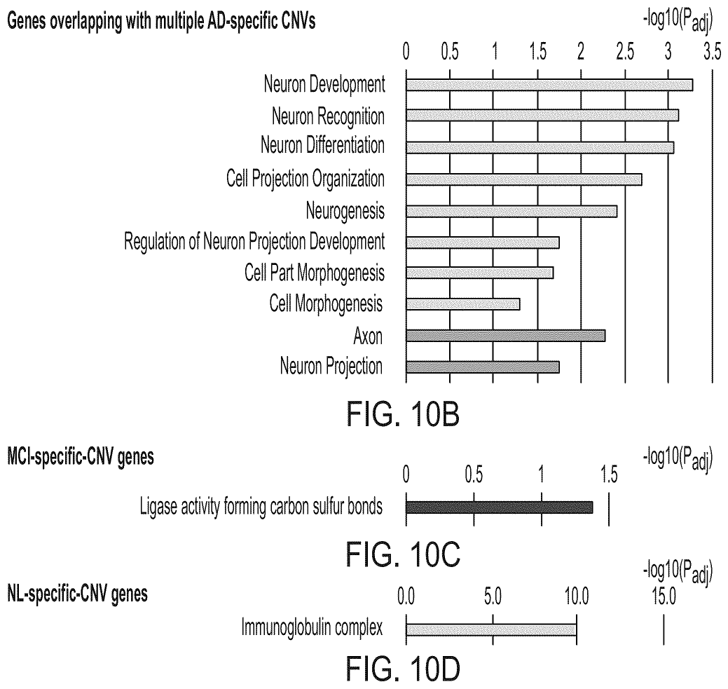

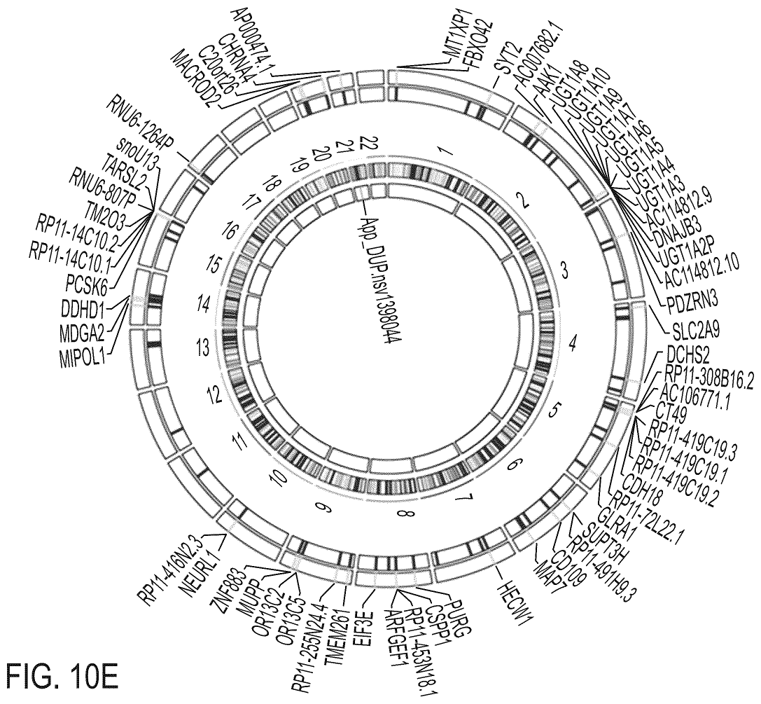

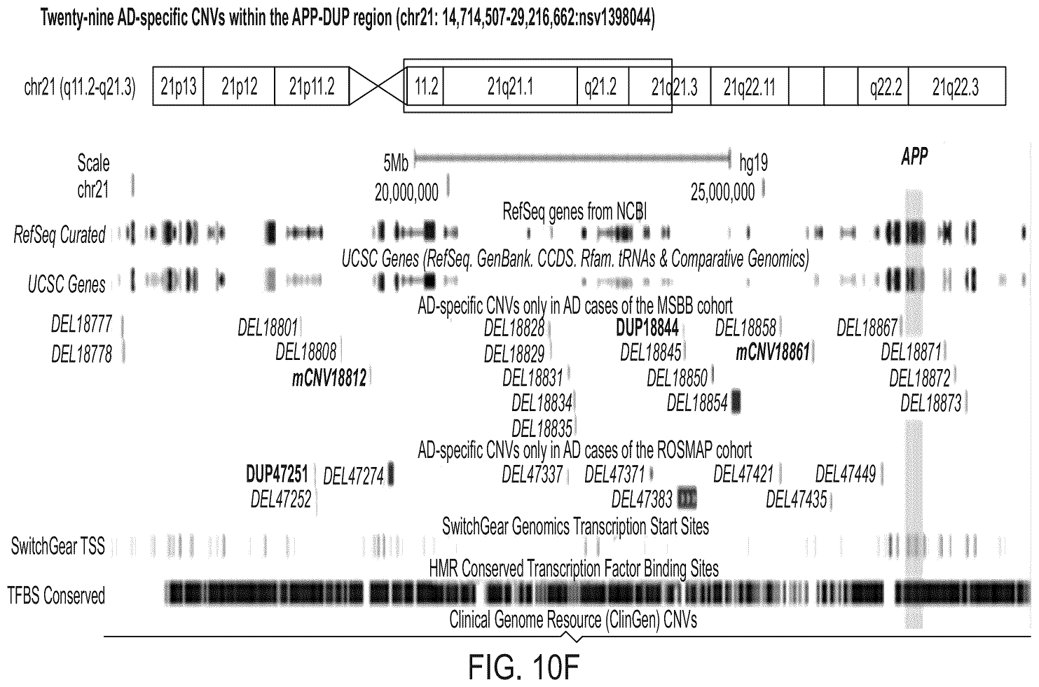

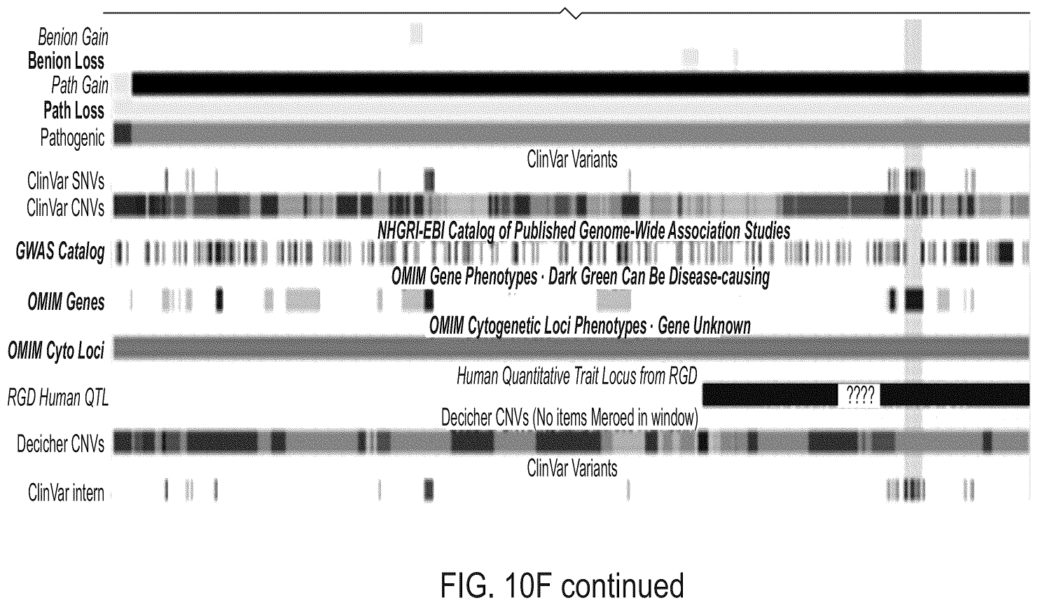

Among these AD-specific CNVs, 64 were conserved in the two cohorts (FIG. 9C, FIG. 10E). The AD-specific CNVs were observed at low population frequencies (6.25% in MSBB, <1.26% in ROSMAP, FIG. 9D). There was no significant difference in the total CNV length or the total CNV count per individual between the AD, MCI, and NL groups in MSBB or ROSMAP, based on the Quasipoisson regression model (QPRM). In MSBB, the mean number (17.19) of the AD-specific CNVs per AD case is significantly higher than that (6.7) of the MCI-specific CNVs per MCI case (QPRM P_{adj}=5*E⁻²) and that (6.64) of the NL-specific CNVs per NL case (QPRM P_{adj}=2.67*E⁻²). The inventors discovered a similar trend in ROSMAP. In QPRM, the clinical diagnostic group is the main predictor variable, the response variable is “the total CNV count” or “the total CNV length” or “the Group-specific CNV count” per individual, while sex and age of death are co-variants.

The inventors discovered that one of the sixty-four AD-specific CNVs conserved across the two cohorts resides within the duplication region encompassing the APP gene (chr21: 14,714,507-29,216,662: nsv1398044) (FIG. 10E). The other sixty-three conserved AD-specific CNVs have not been associated with AD and thus are novel (FIG. 10E). Surprisingly, the majority of these conserved AD-specific CNVs (61 out of 64) are reported in other published CNV datasets, which are based on large populations without mental or neuropathological trait records (i.e., Decipher, DGV, the 1000 Genome project, and GnomAD). The inventors discovered that their frequency is much higher in the AD group than the general population with European ancestry based on the GnomAD database.

Genes whose transcriptional regions reside in the genomic regions of AD-specific CNVs are defined as AD-CNV genes in the subsequent analyses. The inventors discovered that the AD-CNV genes are significantly enriched for important biological processes such as cellular glucuronidation, neuron projection, uronic acid metabolic process, extrinsic component of plasma membrane, synapse, catenin complex, and multicellular organismal signaling (FIG. 10A, Table 7). Furthermore, the inventors found that genes overlapping with multiple AD-specific CNVs are enriched in many neuron-related pathways such as neuron development, neuron recognition, neuron differentiation, cell projection organization, neurogenesis, axon, and neuron projection (FIG. 10B, Table 7). The genes residing in the genomic regions of the MCI-specific CNVs (termed MCI-CNV Genes) are associated with ligase activity forming carbon-sulfur bonds (FIG. 10C, Table 7). In contrast, the genes residing in the genomic regions of the NL-specific CNVs (termed NL-CNV Genes) are enriched for immunoglobulin complex (FIG. 10D, Table 7). These results reveal distinct molecular functions of AD- and MCI-specific CNVs compared to the NL-specific ones.

TABLE 7
Pathways enriched in the group-specific CNV-genes

| | | | | Fold |
|---|---|---|---|---|
| | | | | Enrich- |
| Group | GO term | FET_P | P_{adj} | ment |
| AD-CNV | Plasma Membrane | 1.85E−09 | 5.80E−05 | 1.67 |
| Genes | Region | | | |
| | Flavonoid | 3.13E−09 | 9.80E−05 | 13.39 |
| | Glucuronidation | | | |
| | Cellular | 1.02E−08 | 3.20E−04 | 8.29 |
| | Glucuronidation | | | |
| | Xenobiotic | 5.08E−08 | 1.60E−03 | 10.96 |
| | Glucuronidation | | | |
| | Neuron Projection | 8.00E−08 | 2.50E−03 | 1.56 |
| | Uronic Acid Metabolic | 1.98E−07 | 6.20E−03 | 6.63 |
| | Process | | | |
| | Extrinsic Component of | 2.12E−07 | 6.60E−03 | 2.76 |
| | Plasma Membrane | | | |
| | Synapse | 3.78E−07 | 1.20E−02 | 1.54 |
| | Catenin Complex | 4.51E−07 | 1.40E−02 | 5.65 |
| | Multicellular | 5.27E−07 | 1.60E−02 | 2.52 |
| | Organismal | | | |
| | Signaling | | | |
| | Glucuronosyltransferase | 9.67E−07 | 3.00E−02 | 5.32 |
| | Activity | | | |
| | Synaptic Membrane | 1.09E−06 | 3.40E−02 | 2.05 |
| | Flavonoid Metabolic | 1.56E−06 | 4.90E−02 | 8.04 |
| | Process | | | |
| Genes | Neuron Development | 5.06E−08 | 5.30E−04 | 3.17 |
| overlapping | Neuron Recognition | 7.36E−08 | 7.70E−04 | 19.09 |
| with | Neuron Differentiation | 8.38E−08 | 8.70E−04 | 2.87 |
| multiple | Cell Projection | 1.98E−07 | 2.10E−03 | 2.64 |
| AD- | Organization | | | |
| specific | Neurogenesis | 3.85E−07 | 4.00E−03 | 2.57 |
| CNVs | Axon | 5.18E−07 | 5.40E−03 | 3.85 |
| | Neuron Projection | 1.75E−06 | 1.80E−02 | 2.66 |
| | Regulation Of Neuron | 1.77E−06 | 1.80E−02 | 4.40 |
| | Projection Development | | | |
| | Cell Part Morphogenesis | 2.05E−06 | 2.10E−02 | 3.51 |
| | Cell Morphogenesis | 4.76E−06 | 5.00E−02 | 2.87 |
| MCI-CNV | Ligase Activity Forming | 1.31E−06 | 4.10E−02 | 12.40 |
| Genes | Carbon Sulfur Bonds | | | |
| NL-CNV | Immunoglobulin | 4.14E−15 | 1.30E−10 | 8.45 |
| Genes | Complex | | | |

### Example 11

### Predicting AD Subtypes by Whole Genome Sequencing (WGS) Data

Copy number variations (CNVs) were identified from the WGS data generated from postmortem brain tissues in the MSBB and ROSMAP cohorts, as described earlier. Among 341 MSBB samples, 144 had predicted AD subtypes carrying 19,084 CNVs. Polygenic risk scores (PRS) of SNPs, as well as 22,296 pathway-based and 307 module-based polygenic risk enrichment scores were generated.

For classification and evaluation of classifier performance, the random forest algorithm with caret R package was used. 10-fold cross validation was conducted using the createFolds function and applied automatic tuning with 5 randomly generated parameters. The performance was evaluated by taking the mean of scores collected from 10 iterations.

Data preprocessing and feature selection was applied using only the training data in each iteration. First, preProcess function from the R caret package was used to center and scale the training data and to exclude near zero-variance predictors. Then, the Recursive Feature Elimination (RFE) method was used to select most relevant features in the training set. The RFE algorithm was applied using repeated k-fold cross-validation with three repeats and 5 folds. Different feature sizes (2000, 2100, 2200, . . . , 4000) were evaluated.

To avoid overfitting, the inventors applied 10-fold cross validation. In each iteration, 2000-3600 relevant genetic features were selected by performing feature selection. These selected features have been used to build random forest classifiers. The mean accuracy was estimated as 0.70. The inventors found Area Under the Curve (AUC) values of 0.82, 0.74, and 0.65 for intermediate, typical, and atypical classes, respectively.

### Example 12

### Predicting AD Molecular Subtypes Using Blood Gene Expression Data

RNA sequencing libraries were prepared from peripheral blood mononuclear cells based on the CD14+CD16− markers of AD individuals. Among the sequenced samples, 102 of them have matched brain tissues with AD pathology in the ROSMAP database. The gene expression levels were normalized for different library size by TMM method of edgeR, and adjusted co-variates including MMSE, ExonicRate, SEX, study and Batch by a liner model. The standardized expression levels of different features were used as the input for the machine learning tools. Different parameters were evaluated during the feature selection and model training. Different features sizes (50, 100, 200 and 500) were chosen based on random forest important scores or differentially expressed genes (DEGs, p-value<0.05) between AD subtypes. During the model training, the whole dataset was split into training and testing datasets based on the rules of the K fold cross-validation. Three K values (5, 10, 15) were used for evaluation. For each fold of the cross-validation experiment, nine methods were used to fit the training dataset and the accuracy of the testing dataset was calculated. Finally, the mean accuracy and the standard deviation were calculated from the K-fold experiments. Nine different machine learning methods were implemented including random forest, AdaBoost, logistic regression, decision tree, nearest neighbors (KNN), support vector machines (SVM), naïve Bayes, multi-layer perceptron, and an Ensemble method with equal weights for each classifier.

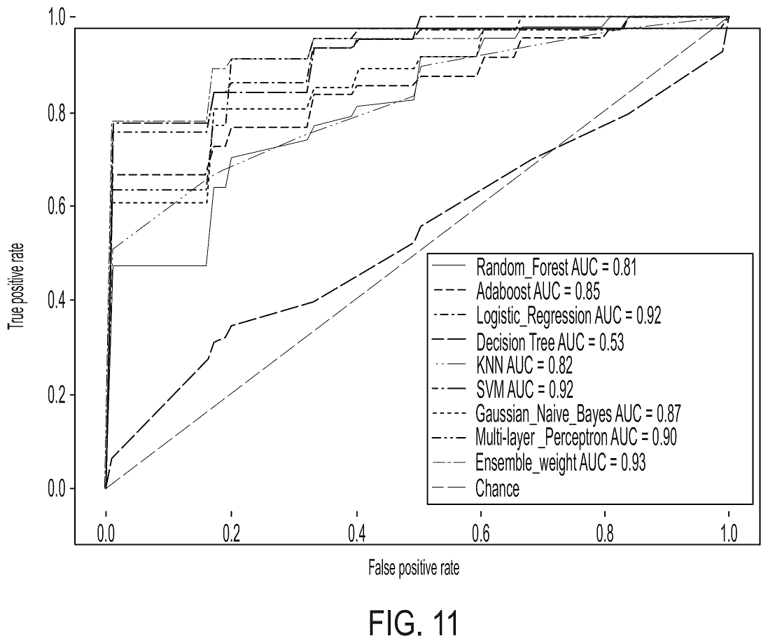

Based on the cross-validation results from different parameter combinations, the inventors identified several methods that can predict AD subtypes using the monocyte gene expressions. The inventors found that the logistic regression, SVM, and Ensemble methods can achieve 0.84 accuracy with 100 or 200 features selected by random forest importance. They found that the multi-layer perceptron and naïve Bayes methods can reach 0.80 accuracy in predicting the AD subtypes. In contrast, the feature selection based on DEGs only achieved as high as 0.63 accuracy for the different methods. The Receiver Operator Characteristic (ROC) curve showed that the logistic regression, SVM, and Ensemble methods have the Area Under the Curve (AUC) values over 0.92 with 200 features selected by random forest, followed by multi-layer perceptron and naïve Bayes with AUC values over 0.85. As shown in FIG. 11, the methods of the present disclosure can accurately predict AD subtypes based on blood monocyte transcriptomes.

### Example 13

### Identification of FDA Approved Drugs for Treating AD Subtypes by Prioritizing Key Network Drivers of Subtypes

For each AD subtype signature, log 2 fold change (logFC) of a gene was weighted by the number of genes that were predicted to be regulated by the key driver (KD) gene through the network KD analysis (KDA) of the subtype signature. Where n genes are predicted to be regulated by a KD gene i in the KDA, the weight is calculated as follows:

$$W = \{ \begin{matrix}2 \sqrt[4]{n} , & if i ∈ KD \\ 1 , & otherwise\end{matrix}$$

Drug-induced signatures of neural progenitor cells (NPC) were identified from the NIH Library of Integrated Network-Based Cellular Signatures (LINCS) Phase I and Phase II datasets. Normalized level 3 data were downloaded from the LINCS data portal. Batch effects of gene expression samples treated with compounds or DMSO from multiple batches were corrected to remove systematic biases. In total, drug-induced signatures were identified for 3,629 compound candidates.

Drug candidate scores between LINCS drug and weighted AD subtype signatures were calculated by the EMUDRA algorithm as previously described (Zhou, et al. 2018). The algorithm uses an ensemble approach of four distinct drug repositioning algorithms (cosine similarity, expression weighted cosines, eXtreme Pearson correlation, eXtreme Spearman rank-ordered correlation). Scores for each method were further processed by a normalization. Normalized scores from the 4 methods were combined into a final score. Drug annotation data was derived from deposited information across the DrugBank database.

For each subtype signature in each of the MSBB and ROSMAP cohorts, a candidate drug was nominated if its EMUDRA matching scores were less than −3 in both LINCS Phase I and Phase II datasets. To further reduce the false positives and ensure robustness of predicted drugs, the analysis required that a drug should be nominated based on both MSBB and ROSMAP cohorts. Using this stringent nomination process, the inventors identified 53 FDA approved drugs targeting 1 or multiple AD subtypes. The inventors further excluded highly toxic oncology drugs and drugs unsuitable for oral administration, leading to 46 drugs, shown in Table 8.

TABLE 8
FDA Approved Drugs that Are Predicted to Be Effective Against AD Molecular Subtypes.

| FDA-approved | | | | | | Multiple | Mechanism of | Disease | |
|---|---|---|---|---|---|---|---|---|---|
| Drug | C1 | C2 | B1 | B2 | A | Subtypes | action (MOA) | Area | Indication |
| thioproperazine | 1 | 1 | | 1 | 1 | 4 | dopamine | neurology/ | schizophrenia |
| | | | | | | | receptor | psychiatry | |
| | | | | | | | antagonist | | |
| nalbuphine | 1 | 1 | | 1 | 1 | 4 | opioid receptor | neurology/ | pain relief |
| | | | | | | | agonist, opioid | psychiatry | |
| | | | | | | | receptor | | |
| | | | | | | | antagonist | | |
| gabexate | 1 | 1 | | 1 | | 3 | serine protease | gastroenterology | pancreatitis |
| | | | | | | | inhibitor | | |
| mesoridazine | | 1 | | 1 | 1 | 3 | dopamine | neurology/ | schizophrenia |
| | | | | | | | receptor | psychiatry | |
| | | | | | | | antagonist | | |
| carbamazepine | 1 | 1 | | | | 2 | carboxamide | neurology/ | seizures |
| | | | | | | | antiepileptic | psychiatry | |
| dimercaptosuccinic- | 1 | | | 1 | | 2 | chelating agent | neurology/ | metal toxicity |
| acid | | | | | | | | psychiatry | |
| menadione | | 1 | | 1 | | 2 | mitochondrial | gastroenterology, | ulcerative |
| | | | | | | | DNA | neurology/ | colitis, |
| | | | | | | | polymerase | psychiatry, | diarrhea, |
| | | | | | | | inhibitor, | dermatology, | headache, |
| | | | | | | | phosphatase | rheumatology | varicose |
| | | | | | | | inhibitor | | veins, |
| | | | | | | | | | rheumatoid |
| | | | | | | | | | arthritis |
| diphenidol | | 1 | | 1 | | 2 | acetylcholine | neurology/ | vertigo |
| | | | | | | | receptor | psychiatry | |
| | | | | | | | agonist | | |
| epirizole | | 1 | | 1 | | 2 | cyclooxygenase | neurology/ | pain relief |
| | | | | | | | inhibitor | psychiatry | |
| timolol | | 1 | | 1 | | 2 | adrenergic | ophthalmology | ocular |
| | | | | | | | receptor | | hypertension, |
| | | | | | | | antagonist | | glaucoma |
| mestranol | | 1 | | 1 | | 2 | estrogen | endocrinology | contraceptive |
| | | | | | | | receptor | | |
| | | | | | | | agonist | | |
| naphazoline | | 1 | | 1 | | 2 | adrenergic | ophthalmology | eye irritation |
| | | | | | | | receptor | | |
| | | | | | | | agonist | | |
| hesperidin | | 1 | | 1 | | 2 | flavanone | | |
| | | | | | | | glycoside | | |
| ethisterone | | | | 1 | 1 | 2 | progestogen | | |
| | | | | | | | hormone | | |
| amlodipine | | 1 | | | | 1 | calcium | cardiology | hypertension, |
| | | | | | | | channel | | chronic stable |
| | | | | | | | blocker | | angina, |
| | | | | | | | | | vasospastic |
| | | | | | | | | | angina, |
| | | | | | | | | | coronary |
| | | | | | | | | | artery disease |
| | | | | | | | | | (CAD) |
| amsacrine | | 1 | | | | 1 | topoisomerase | hematologic | acute |
| | | | | | | | inhibitor | malignancy | lymphoblastic |
| | | | | | | | | | leukemia |
| | | | | | | | | | (ALL), acute |
| | | | | | | | | | lymphoblastic |
| | | | | | | | | | leukemia |
| | | | | | | | | | (ALL) |
| febuxostat | | 1 | | | | 1 | xanthine | nephrology | hyperuricemia |
| | | | | | | | oxidase | | |
| | | | | | | | inhibitor | | |
| famciclovir | | 1 | | | | 1 | DNA | dental, | cold sore, |
| | | | | | | | polymerase | infectious | genitial |
| | | | | | | | inhibitor | disease | herpes, |
| | | | | | | | | | shingles |
| ezetimibe | | 1 | | | | 1 | cholesterol | endocrinology, | hyperlipidemia, |
| | | | | | | | inhibitor, | metabolism | hypercholesterolemia, |
| | | | | | | | Niemann-Pick | | sitosterolemia |
| | | | | | | | C1-like 1 | | |
| | | | | | | | protein | | |
| | | | | | | | antagonist | | |
| carbetocin | | 1 | | | | 1 | oxytocin | hematology | hemorrhage |
| | | | | | | | receptor | | |
| | | | | | | | agonist | | |
| orphenadrine | | | | 1 | | 1 | acetylcholine | neurology/ | muscle pain |
| | | | | | | | receptor | psychiatry | |
| | | | | | | | antagonist | | |
| hyoscyamine | | | | 1 | | 1 | acetylcholine | gastroenterolog | peptic ulcer |
| | | | | | | | receptor | urology, | disease |
| | | | | | | | antagonist | neurology/ | (PUD), acute |
| | | | | | | | | psychiatry, | abdominal |
| | | | | | | | | allergy | visceral |
| | | | | | | | | | spasm, |
| | | | | | | | | | ulcerative |
| | | | | | | | | | colitis, |
| | | | | | | | | | interstitial |
| | | | | | | | | | cystitis (IC), |
| | | | | | | | | | enterocolitis, |
| | | | | | | | | | irritable bowel |
| | | | | | | | | | syndrome, |
| | | | | | | | | | tremors, |
| | | | | | | | | | allergic rhinitis |
| amiodarone.hcl | | | | 1 | | 1 | potassium | cardiology | ventricular |
| | | | | | | | channel | | arrhythmias |
| | | | | | | | blocker | | |
| erythromycin- | | | | 1 | | 1 | cytochrome | infectious | listeria, |
| ethylsuccinate | | | | | | | P450 inhibitor, | disease | respiratory |
| | | | | | | | protein | | tract |
| | | | | | | | synthesis | | infections, |
| | | | | | | | inhibitor | | skin |
| | | | | | | | | | infections, |
| | | | | | | | | | syphilis, |
| | | | | | | | | | amebiasis, |
| | | | | | | | | | pelvic |
| | | | | | | | | | inflammatory |
| | | | | | | | | | disease, |
| | | | | | | | | | chlamydia, |
| | | | | | | | | | diphtheria, |
| | | | | | | | | | erythrasma |
| meclizine | | | | 1 | | 1 | constitutive | gastroenterology, | nausea, |
| | | | | | | | androstane | neurology/ | vomiting, |
| | | | | | | | receptor (CAR) | psychiatry | motion |
| | | | | | | | agonist | cardiology | sickness |
| dobutamine | | | | 1 | | 1 | adrenergic | | congestive |
| | | | | | | | receptor | | heart failure |
| | | | | | | | agonist | | |
| phenazopyridine | | | | 1 | | 1 | local anesthetic | infectious | urinary tract |
| | | | | | | | | disease | infections |
| spironolactone | | | | 1 | | 1 | mineralocorticoid | endocrinology, | hyperaldosteronism, |
| | | | | | | | receptor | cardiology, | congestive |
| | | | | | | | antagonist | gastroenterology, | heart failure, |
| | | | | | | | | rheumatology | hepatic |
| | | | | | | | | | cirrhosis, |
| | | | | | | | | | nephrotic |
| | | | | | | | | | syndrome, |
| | | | | | | | | | hypertension, |
| | | | | | | | | | hypokalemia |
| meclofenamic- | | | | 1 | | 1 | cyclooxygenase | rheumatology, | joint pain, |
| acid | | | | | | | inhibitor, | neurology/ | muscle pain, |
| | | | | | | | prostanoid | psychiatry, | rheumatoid |
| | | | | | | | receptor | endocrinology | arthritis, |
| | | | | | | | antagonist | | primary |
| | | | | | | | | | dysmenorrhea |
| | | | | | | | | | (PD) |
| parachlorophenol | | | | 1 | | 1 | antiinfective | | |
| | | | | | | | drug | | |
| bemegride | | | | 1 | | 1 | chemoreceptor | critical care | poison |
| | | | | | | | agonist | | antidote |
| ketorolac | | | | 1 | | 1 | cyclooxygenase | | |
| | | | | | | | inhibitor | | |
| brinzolamide | | | | 1 | | 1 | carbonic | ophthalmology | intraocular |
| | | | | | | | anhydrase | | pressure, |
| | | | | | | | inhibitor | | glaucoma, |
| | | | | | | | | | ocular |
| | | | | | | | | | hypertension |
| nortriptyline | | | | 1 | | 1 | tricyclic | neurology/ | depression |
| | | | | | | | antidepressant | psychiatry | |
| hexylcaine | | | | 1 | | 1 | sodium | neurology/ | local |
| | | | | | | | channel | psychiatry | anesthetic |
| | | | | | | | blocker | | |
| omeprazole | | | | 1 | | 1 | ATPase | gastroenterology | heartburn |
| | | | | | | | inhibitor | | |
| norgestrel | | | | 1 | | 1 | progesterone | endocrinology | contraceptive |
| | | | | | | | receptor | | |
| | | | | | | | agonist | | |
| olmesartan- | | | | 1 | | 1 | angiotensin | cardiology | hypertension |
| medoxomil | | | | | | | receptor | | |
| | | | | | | | antagonist | | |
| perphenazine | | | | 1 | | 1 | dopamine | neurology/ | schizophrenia, |
| | | | | | | | receptor | psychiatry, | nausea, |
| | | | | | | | antagonist | gastroenterology | vomiting |
| promazine | | | | 1 | | 1 | dopamine | neurology/ | schizophrenia |
| | | | | | | | receptor | psychiatry | |
| | | | | | | | antagonist | | |
| metolazone | | | | 1 | | 1 | carbonic | cardiology | edema, |
| | | | | | | | anhydrase | | hypertension |
| | | | | | | | inhibitor | | |
| citalopram | | | 1 | | | 1 | selective | neurology/ | depression |
| | | | | | | | serotonin | psychiatry | |
| | | | | | | | reuptake | | |
| | | | | | | | inhibitor (SSRI) | | |
| clonazepam | | | 1 | | | 1 | GABA | neurology/ | seizures, |
| | | | | | | | benzodiazepine | psychiatry | panic |
| | | | | | | | site receptor | | disorders |
| | | | | | | | agonist | | |
| lamotrigine | | | 1 | | | 1 | serotonin | neurology/ | epilepsy, |
| | | | | | | | receptor | psychiatry | bipolar |
| | | | | | | | antagonist, | | disorder |
| | | | | | | | sodium | | |
| | | | | | | | channel | | |
| | | | | | | | blocker | | |
| mosapride | | | | | 1 | 1 | serotonin | gastroenterology | hypertrophic |
| | | | | | | | receptor | | gastritis |
| | | | | | | | agonist | | (GHG), |
| | | | | | | | | | gastroesophageal |
| | | | | | | | | | reflux |
| | | | | | | | | | disease |
| | | | | | | | | | (GERD), |
| | | | | | | | | | dyspepsia, |
| | | | | | | | | | irritable bowel |
| | | | | | | | | | syndrome |
| ephedrine | | | | | 1 | 1 | adrenergic | cardiology, | hypotension, |
| | | | | | | | receptor | pulmonary, | asthma, |
| | | | | | | | agonist | neurology/ | narcolepsy, |
| | | | | | | | | psychiatry, | obesity |
| | | | | | | | | endocrinology | |

*1 in the columns 2 to 6 indicates a drug is predicted to be effective for a subtype. The column 7 is the sum of the columns 2 to 6.

Although some of these predicted drugs have been tested in the context of AD, none was tested in the context of AD molecular subtypes.

As shown in Table 8, fourteen of the 46 predicted drugs are predicted to be effective for multiple subtypes but none of them is effective for all the five subtypes.

The inventors discovered that two drugs thioproperazine and nalbuphine can target all subtypes except the intermediate subtype B1. Thioproperazine is an antipsychotic indicated for the management of acute and chronic schizophrenia, including cases that are refractory to more common neuroleptics. Thioproperazine is used for treatment of behavioral and psychological symptoms in older people with dementia. Nalbuphine is an opioid analgesic which is used in the treatment of pain. It acts as a moderate-efficacy partial agonist or antagonist of the μ-opioid receptor (MOR) and as a high-efficacy partial agonist of the κ-opioid receptor (KOR), whereas it has relatively low affinity for the δ-opioid receptor (DOR) and sigma receptors. It is prescribed in older adults with or without Alzheimer disease and related dementia.

The inventors discovered that two drugs Gabexate and Mesoridazine can target three AD subtypes. Gabexate, targeting subtypes C1, C2 and B2, is a synthetic serine protease inhibitor which has been used as an anticoagulant. It also known to decrease production of inflammatory cytokines. Gabexate has been investigated for use in cancer, ischemia-reperfusion injury, and pancreatitis. Gabexate also functions as a small molecular inhibitor of serine protease, which may be related with Alzheimer's disease (Leung, D. et al. 2000), thereby supporting the inventors' discovery of its usefulness for treating AD subtypes subtypes C1, C2 and B2.

Mesoridazine (Serentil), targeting subtypes C2, B2 and A, is a piperidine neuroleptic drug belonging to the class of drugs called phenothiazines, used in the treatment of schizophrenia. Mesoridazine exhibited potent inhibitory effects on acetylcholinesterase, which is a therapeutic target in the treatment of Alzheimer's disease (Ko, L. et al. 1997), providing additional support for the inventors' discovery of mesoridazine's usefulness for treating AD subtypes C2, B2, and A.

The inventors discovered that Menadione (Vitamin K3) can target two subtypes (C2 and B2); it is a fat-soluble vitamin precursor that is converted into menaquinone in the liver. The primary known function of vitamin K is to assist in the normal clotting of blood, but it may also play a role in normal bone calcification. Menadione causes oxidative stress by generating reactive oxygen species. Menadione-induced tau dephosphorylation in cultured human neuroblastoma cells. Menadione sodium bisulfite inhibits the toxic aggregation of amyloid-3, further supporting the inventors' present disclosure of its utility for treating AD subtypes C2 and B2.

The inventors discovered that Carbamazepine (CBZ) targets subtypes C1 and C2; it is an anticonvulsant medication used primarily in the treatment of epilepsy and neuropathic pain. It is used as an adjunctive treatment in schizophrenia along with other medications and as a second-line agent in bipolar disorder. Carbamazepine may be effective in treating agitation in severely demented Alzheimer's in patients that are refractory to neuroleptic medication alone, particularly those that fall into the subtype C1 and C2 categories.

The inventors discovered six drugs Amlodipine, amsacrine, febuxostat, famciclovir, ezetimibe, and carbetocin that are specifically effective for the typical AD subtype C2. Amlodipine, targeting subtype C2, is a L-type calcium channel blocker used to treat hypertension and angina. Amlodipine is being tested in phase 2/3 trials to reduce the risk for Alzheimer's disease (NCT02913664), but the results have not been disclosed (Schampel, A. & Kuerten, S., 2017). Amlodipine cannot pass the blood-brain barrier and may elicit neuroprotective effects to reverse calcium-induced excitotoxicity and mitochondrial dysfunction that underlie several neurologic disorders including Alzheimer's disease when delivered into the brain through the blood-brain barrier (Alawdi, S. H. et al. 2019), supporting the present discovery of its utility in treating AD, subtype 2.

Febuxostat is a xanthine oxidase/dehydrogenase inhibitor that achieves its therapeutic effect by decreasing serum uric acid. Febuxostat is used for the management of chronic hyperuricemia in adults with gout. The association of uric acid levels and dementia is an emerging area of interest. A dose-related reduction in the risk of dementia in older adults has been shown with febuxostat daily dose (Singh, J. A. et al. 2018), which supports the inventors' present disclosure showing its usefulness for treating AD subtype C2.

Ezetimibe is an anti-hyperlipidemic medication that selectively inhibits the intestinal absorption of cholesterol and related phytosterols. It has a mechanism of action that differs from those of other classes of cholesterol-reducing compounds. Ezetimibe does not inhibit cholesterol synthesis in the liver or increase bile acid excretion, but, rather, it localizes and appears to act at the brush border of the small intestine and inhibits the absorption of cholesterol, leading to a decrease in the delivery of intestinal cholesterol to the liver. This causes a reduction of hepatic cholesterol stores and an increase in clearance of cholesterol from the blood; this distinct mechanism is complementary to that of HMG-CoA reductase inhibitors. High cholesterol levels have been positively correlated with a higher incidence of memory impairment and dementia. Therefore, a study was undertaken to investigate the potential of the ezetimibe in memory deficits associated with dementia of Alzheimer's type in mice (Dalla, Y. et al. 2009). Ezetimibe significantly attenuated streptozotocin-induced memory deficits and biochemical changes in mice. The memory-restorative effect of ezetimibe can be attributed to its cholesterol-dependent as well as cholesterol-independent effects. The prior mouse study, when combined with the inventors' discovery of ezitembe's effectiveness in targeting the typical AD subtype C2, highlights ezetimibe's usefulness in addressing in memory dysfunctions associated with dementia of AD.

The inventors discovered that the intermediate AD subtype B2 is specifically targeted by 21 different drugs. Orphenadrine, for example, is an anticholinergic drug used to treat muscle pain and to help with motor control in Parkinson's disease. It binds and inhibits both histamine H1 receptors and NMDA receptors. In addition, it has mild antihistaminic and local anesthetic properties. Moreover, the protective effect of orphenadrine on glutamate neurotoxicity has been shown in vitro and in vivo. Phenazopyridine is an effective oral urinary analgesic commonly used for the treatment of irritative lower urinary tract conditions. Erythromycin ethylsuccinate is a macrolide antibiotic used to treat and prevent a variety of bacterial infections. Erythromycin provided orally to transgenic mice TgCRND8 has been shown to consistently reduced brain Abeta (1-42) levels. Amiodarone is a class III antiarrhythmic. It blocks sodium channels at rapid pacing frequencies, and like class II drugs, amiodarone exerts a noncompetitive antisympathetic action. In addition to blocking sodium channels, amiodarone blocks myocardial potassium channels, which contributes to slowing of conduction and prolongation of refractoriness. It is indicated for initiation of treatment and prophylaxis of frequently recurring ventricular fibrillation and hemodynamically unstable ventricular tachycardia in patients that are refractory to other therapy. Hyoscyamine, the levo-isomer of atropine, is an anticholinergic and a natural plant alkaloid derivative. Hyoscyamine is indicated to treat various gastrointestinal problems such as cramps and irritable bowel syndrome. Hyoscyamine is used for various treatments and therapeutics due to its antimuscarinic properties, such as bladder and bowel control problems, cramping pain caused by kidney stones and gallstones, and Parkinson's disease. In addition, it is used to decrease the side effects of certain medications and insecticides.

The inventors discovered that the intermediate AD subtype B1 is specifically targeted by three drugs (Citalopram, Clonazepam, and lamotrigine). Citalopram is a selective serotonin reuptake inhibitor (SSRI) used in the treatment of depression. It potentiates serotonergic activity in the central nervous system (CNS) due to its inhibition of CNS neuronal reuptake of serotonin (5-HT). The molecular target for citalopram is the serotonin transporter (SLC6A4), inhibiting its serotonin reuptake in the synaptic cleft. In vitro and in vivo studies in animals suggest that citalopram binds with significantly less affinity to histamine, acetylcholine, and norepinephrine receptors than tricyclic antidepressant drugs. Besides its FDA-approved indication for the treatment of depression, citalopram can be used off-label for the treatment of sexual dysfunction, post-stroke behavioral changes, ethanol abuse, obsessive-compulsive disorder in children, diabetic neuropathy, and many others. Additionally, citalopram is used in the treatment of agitation in Alzheimer's disease (Aga, V. M. 2019).

Serotonin is an important neurotransmitter that participates in the modulation of memory formation. The decreased levels of both serotonin and its receptors have been reported in human post-mortem AD studies (Xu, Y. et al. 2012). Serotonin reduces generation of amyloid-β in vitro and in animal models of AD (Cirrito, J. R. et al. 2011). As an SSRI drug, citalopram is indicated in lowering brain Aβ concentrations. It reduced Aβ formation and decreased the release of the proinflammatory factors of activated microglia in vitro (Dhami, K. S. et al. 2013). Chronic citalopram treatment reduced Aβ plaque load in APP/PS1 mice. Citalopram, promoting synaptic plasticity and hippocampal neurogenesis, could also improve learning and memory in social isolation rats (Gong, W. G. et al 2017). Moreover, an acute dose of citalopram administration has been linked to a decreased amount of newly generated Aβ in young healthy humans (Sheline et al., 2014). It is inferred that citalopram has beneficial effects on memory deficit and non-cognitive neuropsychiatric behaviors in treating AD, although the underlying mechanism remains unclear. Recently, Zhang et al. showed that chronic citalopram administration in APP/PS1 mice could rescue impaired short-term memory and ameliorate non-cognitive behavioral deficits as well as decreased the amyloid plaque load in the brain (Zhang et al. 2018). These prior findings support the inventors' present disclosure showing citalopram's utility in the early treatment of AD, particularly subtype B1.

Clonazepam is a long-acting benzodiazepine that can bind to benzodiazepine receptors, which are components of various varieties of gamma-aminobutyric acid (GABA) receptors, thereby potentiating the effects of GABA (DeVane et al. 1991). As GABA is an inhibitory neurotransmitter, this results in increased inhibition of the ascending reticular activating system. Clonazepam, in this way, facilitates various effects like sedation, hypnosis, skeletal muscle relaxation, anticonvulsant activity, and anxiolytic action (Nardi et al. 2013). The agent has been indicated for treating panic disorders, severe anxiety, and various seizures. Although it was shown that clonazepam might potentially reduce microglial neuroinflammation, there were no replicated findings for benzodiazepines (Wilms, H. et al. 2003). The present disclosure indicates that clonazepam would be useful for the treatment of AD, specifically subtype B1.

Lamotrigine is a phenyltriazine antiepileptic used to treat some types of epilepsy and bipolar disorder. Lamotrigine could have some clinical efficacy in certain neuropathic pain states, as well (Jensen, T. S. 2002 & Pappagallo, M. 2003). While the precise mechanism by which lamotrigine exerts its anticonvulsant action are unknown, one proposed mechanism of action of lamotrigine involves an effect on sodium channels. In vitro pharmacological studies demonstrate that lamotrigine inhibits voltage-sensitive sodium channels, thereby stabilizing neuronal membranes and consequently modulating the presynaptic release of excitatory amino acid transmitters. Lamotrigine is used to treat seizures associated with Alzheimer's disease (Vossel, K. A. et al. 2017 & Wu, H. et al. 2015). In addition, it was shown that lamotrigine could ameliorate executive dysfunction and brain inflammatory response in the mouse model of AD. In combination with the inventors' present disclosure showing lamotrigine's utility for targeting AD, subtype B1, early lamotrigine intervention is a therapeutic strategy for AD.

The inventors discovered that atypical AD subtype A is specifically targeted by Mosapride and Ephedrine. Mosapride is a prokinetic serotonin 5HT₄-receptor agonist and serotonin 5HT₃-receptor antagonist, which stimulates gastric motility. This drug is used clinically to treat gastrointestinal motility disorders. Ephedrine is an alpha and beta-adrenergic agonist which indirectly increases the release of norepinephrine from sympathetic neurons. In combination, these actions lead to larger quantities of norepinephrine present in the synapse, for longer periods of time, increasing stimulation of the sympathetic nervous system. As a sympathomimetic amine, ephedrine has vasoconstrictive, positive chronotropic, and positive inotropic effects. Ephedrine crosses the blood brain barrier and stimulates the central nervous system. Ephedrine products are now banned in many countries, as they are a major source for the production of the addictive compound methamphetamine. The FDA has approved ephedrine only for the treatment of clinically important hypotension occurring in the setting of anesthesia, but the present disclosure shows its utility for treating atypical AD subtype A.

### Example 14

### Identification of Drugs for Treating AD Subtypes by Reversing Subtype Gene Signatures

An in silico EMUDRA analysis was performed to match differential expression signatures from each subtype class with known drug differential expression signatures. AD subtype differential expression signatures were generated from the RNA-seq data previously described during molecular subtype characterization, using the log fold-change for each significant DEG as input to EMUDRA. The LINCS L1000 gene expression drug signature dataset of 3,629 therapeutic candidates in neural progenitor cells (NPCs) was also used. EMUDRA analysis was then performed to determine if any drugs are beneficial (e.g., drug DEGs are opposite the direction of subtype DEG signature) or detrimental (e.g., drug DEGs are similar in direction to the subtype DEG signature). After EMUDRA processing, additional annotation was performed by DrugBank database matching to identify each drug's common name and mechanism of action (MOA).

The inventors identified 1,126 drugs with predicted beneficial effect for class A, 966 for class B, and 1,035 drugs for class B, under an adjusted q-value of <0.05; the top 10 per subclass are identified in Table 9. The inventors further discovered that 94 of the drugs were categorized as beneficial for classes A and B, 70 for classes A and C, and 273 for classes B and C. Using this analytical framework, the inventors did not find any drugs predicted to be beneficial for all subtype classes. EMUDRA analysis was also repeated for each of the five individual AD subtypes. The inventors discovered 1046 beneficial drugs for subtype A, 931 for B1, 1034 for B2, 1003 for C1, and 1077 for C2; the top ten drugs for each of the AD subtypes are provided in Table 10.

TABLE 9
Top 10 Drugs Predicted for each AD Subtype Class

| | | Class A | | | Class B | | | Class C | | |
|---|---|---|---|---|---|---|---|---|---|---|
| | | Norm. | | | Norm. | | | Norm. | | |
| Drug Name | MOA | Score | P | Q | Score | P | Q | Score | P | Q |
| GW-3965 | LXR agonist | −8.79 | 1.53E−18 | 3.64E−16 | 3.39 | 6.99E−04 | 1.01E−03 | 6.81 | 9.75E−12 | 1.50E−10 |
| ciglitazone | PPAR receptor agonist | −8.16 | 3.35E−16 | 2.45E−14 | 3.36 | 7.67E−04 | 1.09E−03 | 6.21 | 5.37E−10 | 5.33E−09 |
| | glutamate receptor | | | | | | | | | |
| L-689560 | antagonist | −7.48 | 7.46E−14 | 2.32E−12 | 5.68 | 1.36E−08 | 1.09E−07 | 4.17 | 3.09E−05 | 6.61E−05 |
| | glucocorticoid receptor | | | | | | | | | |
| fludroxycortide | agonist | −6.89 | 5.52E−12 | 1.05E−10 | 2.08 | 3.78E−02 | 2.84E−02 | 4.48 | 7.45E−06 | 2.01E−05 |
| sirtinol | SIRT inhibitor | −6.83 | 8.56E−12 | 1.45E−10 | 1.83 | 6.66E−02 | 4.54E−02 | 7.46 | 8.75E−14 | 3.09E−12 |
| | potassium channel | | | | | | | | | |
| Y-26763 | activator | −6.77 | 1.25E−11 | 1.94E−10 | 3.64 | 2.71E−04 | 4.40E−04 | 4.10 | 4.10E−05 | 8.37E−05 |
| mometasone- | glucocorticoid receptor | | | | | | | | | |
| furoate | agonist | −6.77 | 1.29E−11 | 1.98E−10 | 2.30 | 2.15E−02 | 1.79E−02 | 5.05 | 4.54E−07 | 1.79E−06 |
| | carboxamide | | | | | | | | | |
| carbamazepine | antiepileptic | −6.73 | 1.70E−11 | 2.52E−10 | 7.04 | 1.94E−12 | 7.29E−11 | −0.79 | 4.28E−01 | 1.78E−01 |
| erythrosine | coloring agent | −6.70 | 2.13E−11 | 3.02E−10 | 8.90 | 5.56E−19 | 1.83E−16 | 2.68 | 7.38E−03 | 6.66E−03 |
| | serotonin receptor | | | | | | | | | |
| MDL-72832 | agonist | −6.50 | 7.98E−11 | 9.36E−10 | 3.72 | 1.99E−04 | 3.42E−04 | 5.88 | 4.09E−09 | 3.08E−08 |
| NU-1025 | PARP inhibitor | 5.01 | 5.42E−07 | 1.65E−06 | −9.28 | 1.69E−20 | 1.11E−17 | −0.20 | 8.44E−01 | 2.94E−01 |
| | acetylcholine receptor | | | | | | | | | |
| cyclopentolate | antagonist | 5.42 | 5.80E−08 | 2.57E−07 | −7.78 | 7.27E−15 | 9.87E−13 | −3.72 | 1.99E−04 | 3.16E−04 |
| | Abl kinase inhibitor, src | | | | | | | | | |
| | inhibitor, VEGFR | | | | | | | | | |
| ZM-306416 | inhibitor | 0.81 | 4.21E−01 | 1.39E−01 | −7.46 | 8.65E−14 | 7.59E−12 | 1.55 | 1.22E−01 | 6.59E−02 |
| | serotonin receptor | | | | | | | | | |
| CP-93129 | agonist | 6.02 | 1.78E−09 | 1.24E−08 | −7.33 | 2.39E−13 | 1.65E−11 | −1.16 | 2.46E−01 | 1.17E−01 |
| | GABA receptor | | | | | | | | | |
| CGP-13501 | modulator | 3.98 | 6.96E−05 | 1.01E−04 | −7.30 | 2.93E−13 | 1.93E−11 | −0.65 | 5.15E−01 | 2.04E−01 |
| CI-966 | GAT inhibitor | 7.77 | 7.58E−15 | 3.43E−13 | −7.23 | 4.93E−13 | 2.82E−11 | −0.92 | 3.56E−01 | 1.55E−01 |
| FK-888 | tachykinin antagonist | 8.03 | 1.01E−15 | 5.66E−14 | −7.11 | 1.15E−12 | 5.62E−11 | −4.32 | 1.53E−05 | 3.73E−05 |
| | estrogen receptor | | | | | | | | | |
| PPT | agonist | 1.29 | 1.99E−01 | 7.63E−02 | −7.04 | 1.93E−12 | 7.29E−11 | 5.26 | 1.46E−07 | 6.76E−07 |
| cyclosporin-a | calcineurin inhibitor | 4.74 | 2.14E−06 | 5.44E−06 | −7.02 | 2.25E−12 | 8.00E−11 | −0.13 | 8.93E−01 | 3.06E−01 |
| clofibric acid | PPAR receptor agonist | 2.09 | 3.68E−02 | 1.95E−02 | −6.92 | 4.57E−12 | 1.43E−10 | 1.67 | 9.45E−02 | 5.34E−02 |
| | acetylcholinesterase | | | | | | | | | |
| neostigmine | inhibitor | 8.18 | 2.82E−16 | 2.23E−14 | −4.28 | 1.90E−05 | 4.74E−05 | −10.08 | 6.83E−24 | 4.10E−21 |
| FIT | opioid receptor agonist | 6.78 | 1.20E−11 | 1.91E−10 | −2.03 | 4.19E−02 | 3.10E−02 | −8.84 | 9.69E−19 | 1.16E−16 |
| | histamine receptor | | | | | | | | | |
| ciproxifan | antagonist | 4.68 | 2.94E−06 | 7.15E−06 | −1.82 | 6.81E−02 | 4.62E−02 | −8.30 | 1.02E−16 | 8.74E−15 |
| | dopamine receptor | | | | | | | | | |
| quinpirol-(—) | agonist | 5.67 | 1.42E−08 | 7.71E−08 | −1.64 | 1.02E−01 | 6.40E−02 | −8.15 | 3.74E−16 | 2.64E−14 |
| | purinergic receptor | | | | | | | | | |
| clopidogrel | antagonist | 7.17 | 7.49E−13 | 1.73E−11 | −1.16 | 2.48E−01 | 1.30E−01 | −8.13 | 4.14E−16 | 2.76E−14 |
| | acetylcholine release | | | | | | | | | |
| DMP-543 | enhancer | 6.50 | 8.23E−11 | 9.50E−10 | −3.92 | 8.81E−05 | 1.74E−04 | −8.07 | 7.27E−16 | 4.59E−14 |
| | adrenergic receptor | | | | | | | | | |
| salmeterol | agonist | 7.76 | 8.71E−15 | 3.76E−13 | −2.57 | 1.01E−02 | 9.50E−03 | −7.34 | 2.07E−13 | 5.77E−12 |
| | acetylcholine receptor | | | | | | | | | |
| tremorine | agonist | 6.61 | 3.94E−11 | 5.12E−10 | −1.58 | 1.13E−01 | 6.96E−02 | −7.29 | 3.08E−13 | 8.23E−12 |
| | acetylcholine receptor | | | | | | | | | |
| piperidolate | antagonist | 8.73 | 2.55E−18 | 4.84E−16 | −5.16 | 2.51E−07 | 1.20E−06 | −7.19 | 6.59E−13 | 1.50E−11 |
| | ATP channel activator, | | | | | | | | | |
| | potassium channel | | | | | | | | | |
| pinacidil | activator | 2.26 | 2.37E−02 | 1.35E−02 | 1.22 | 2.23E−01 | 1.20E−01 | −7.02 | 2.18E−12 | 4.16E−11 |

The DrugBank name, EMUDRA normalized score against LINCS drug signatures evaluated in NPCs (higher is better, negative is a detrimental drug), mechanism of action, p-value, and adjusted q-value fit with the gene differential expression signature for each AD subtype are provided for each of the top 10 drugs per subtype class. A drug must have a negative normalized score and significant (<0.05) q-value to be beneficial drug for a particular subtype class; positive normalized scores are detrimental.

TABLE 10
Top 10 Drugs Predicted for each AD Subtype

| | | Subtype A | | Subtype B1 | | Subtype B2 | | Subtype C1 | | Subtype C2 | |
|---|---|---|---|---|---|---|---|---|---|---|---|
| | | Norm. | | Norm. | | Norm. | | Norm. | | Norm. | |
| Name | MOA | Score | Q | Score | Q | Score | Q | Score | Q | Score | Q |
| GW-3965 | LXR | −8.85 | 2.81E−16 | 0.70 | 2.23E−01 | 3.58 | 4.30E−04 | 5.73 | 7.27E−08 | 7.82 | 3.13E−13 |
| | agonist | | | | | | | | | | |
| ciglitazone | PPAR | −8.28 | 1.42E−14 | 1.23 | 1.24E−01 | 4.30 | 3.42E−05 | 5.28 | 6.59E−07 | 6.09 | 1.16E−08 |
| | receptor | | | | | | | | | | |
| | agonist | | | | | | | | | | |
| L-689560 | glutamate | −7.43 | 4.49E−12 | 4.39 | 3.43E−05 | 6.05 | 1.36E−08 | 1.74 | 5.02E−02 | 7.04 | 4.66E−11 |
| | receptor | | | | | | | | | | |
| | antagonist | | | | | | | | | | |
| sirtinol | SIRT | −6.91 | 1.29E−10 | 1.28 | 1.15E−01 | 3.14 | 1.72E−03 | 6.48 | 1.16E−09 | 7.47 | 3.05E−12 |
| | inhibitor | | | | | | | | | | |
| fludroxycortide | glucocorticoid | −6.87 | 1.56E−10 | 0.98 | 1.68E−01 | 4.06 | 8.36E−05 | 3.18 | 1.84E−03 | 5.91 | 2.84E−08 |
| | receptor | | | | | | | | | | |
| | agonist | | | | | | | | | | |
| erythrosine | coloring | −6.78 | 2.57E−10 | 8.42 | 5.79E−15 | 8.43 | 8.28E−15 | 0.41 | 2.67E−01 | 7.39 | 5.06E−12 |
| | agent | | | | | | | | | | |
| Y-26763 | potassium | −6.70 | 4.22E−10 | 1.47 | 8.72E−02 | 4.51 | 1.57E−05 | 3.75 | 3.14E−04 | 4.95 | 2.44E−06 |
| | channel | | | | | | | | | | |
| | activator | | | | | | | | | | |
| carbamazepine | carboxamide | −6.68 | 4.49E−10 | 5.52 | 2.33E−07 | 5.98 | 1.94E−08 | −3.05 | 2.69E−03 | 4.67 | 8.03E−06 |
| | antiepileptic | | | | | | | | | | |
| mometasone- | glucocorticoid | −6.67 | 4.91E−10 | −0.64 | 2.38E−01 | 4.15 | 6.10E−05 | 4.13 | 8.07E−05 | 6.04 | 1.52E−08 |
| furoate | receptor | | | | | | | | | | |
| | agonist | | | | | | | | | | |
| MDL-72832 | serotonin | −6.51 | 1.21E−09 | 2.05 | 3.23E−02 | 5.21 | 8.15E−07 | 4.59 | 1.28E−05 | 6.31 | 3.38E−09 |
| | receptor | | | | | | | | | | |
| | agonist | | | | | | | | | | |
| BML-284 | WNT | 5.33 | 5.59E−07 | −8.89 | 1.71E−16 | −0.62 | 1.94E−01 | 5.89 | 3.07E−08 | −0.95 | 1.33E−01 |
| | agonist | | | | | | | | | | |
| NU-1025 | PARP | 4.87 | 4.17E−06 | −8.42 | 5.79E−15 | −9.68 | 1.38E−19 | 1.90 | 3.81E−02 | −4.87 | 3.45E−06 |
| | inhibitor | | | | | | | | | | |
| PPT | estrogen | 1.45 | 8.14E−02 | −8.38 | 6.25E−15 | −5.07 | 1.50E−06 | 6.43 | 1.54E−09 | 1.03 | 1.20E−01 |
| | receptor | | | | | | | | | | |
| | agonist | | | | | | | | | | |
| GW-0742 | PPAR | 8.12 | 4.64E−14 | −8.15 | 3.69E−14 | −6.12 | 9.19E−09 | −0.01 | 3.46E−01 | −7.39 | 5.06E−12 |
| | receptor | | | | | | | | | | |
| | agonist | | | | | | | | | | |
| adapalene | retinoid | 2.88 | 4.18E−03 | −7.98 | 1.27E−13 | −1.63 | 5.27E−02 | 5.41 | 3.40E−07 | −2.50 | 8.77E−03 |
| | receptor | | | | | | | | | | |
| | agonist | | | | | | | | | | |
| imatinib | Bcr-Abl | −1.51 | 7.55E−02 | −7.92 | 1.93E−13 | −3.70 | 2.90E−04 | 8.29 | 9.17E−15 | 1.21 | 9.50E−02 |
| | kinase | | | | | | | | | | |
| | inhibitor, | | | | | | | | | | |
| | KIT | | | | | | | | | | |
| | inhibitor, | | | | | | | | | | |
| | PDGFR | | | | | | | | | | |
| | tyrosine | | | | | | | | | | |
| | kinase | | | | | | | | | | |
| | receptor | | | | | | | | | | |
| | inhibitor | | | | | | | | | | |
| CP-93129 | serotonin | 5.96 | 2.31E−08 | −7.69 | 8.32E−13 | −6.73 | 3.49E−10 | 0.67 | 2.15E−01 | −6.42 | 1.81E−09 |
| | receptor | | | | | | | | | | |
| | agonist | | | | | | | | | | |
| proxyfan | histamine | 1.65 | 5.93E−02 | −7.60 | 1.63E−12 | −5.31 | 5.15E−07 | 3.60 | 5.07E−04 | −1.94 | 2.87E−02 |
| | receptor | | | | | | | | | | |
| | modulator | | | | | | | | | | |
| tranylcypromine | monoamine | −1.77 | 4.85E−02 | −7.44 | 4.43E−12 | −5.97 | 2.04E−08 | 5.62 | 1.20E−07 | 1.32 | 8.21E−02 |
| | oxidase | | | | | | | | | | |
| | inhibitor | | | | | | | | | | |
| ilomastat | matrix | 4.24 | 5.21E−05 | −7.38 | 6.54E−12 | −5.72 | 7.05E−08 | −1.14 | 1.26E−01 | −5.36 | 3.88E−07 |
| | metalloprotease | | | | | | | | | | |
| | inhibitor | | | | | | | | | | |
| NU-1025 | PARP | 4.87 | 4.17E−06 | −8.42 | 5.79E−15 | −9.68 | 1.38E−19 | 1.90 | 3.81E−02 | −4.87 | 3.45E−06 |
| | inhibitor | | | | | | | | | | |
| FK-888 | tachykinin | 7.84 | 3.06E−13 | −4.36 | 3.95E−05 | −7.95 | 2.32E−13 | −1.94 | 3.54E−02 | −9.01 | 2.84E−17 |
| | antagonist | | | | | | | | | | |
| phenazopyridine | local | 5.66 | 1.12E−07 | −4.07 | 1.16E−04 | −7.93 | 2.32E−13 | 1.72 | 5.21E−02 | −4.78 | 5.07E−06 |
| | anesthetic | | | | | | | | | | |
| PNU-22394 | serotonin | 7.60 | 1.47E−12 | −6.08 | 1.39E−08 | −7.92 | 2.32E−13 | 0.47 | 2.55E−01 | −8.56 | 1.28E−15 |
| | receptor | | | | | | | | | | |
| | agonist | | | | | | | | | | |
| clofibric acid | PPAR | 1.92 | 3.69E−02 | −3.05 | 3.02E−03 | −7.91 | 2.44E−13 | 2.95 | 3.54E−03 | −2.36 | 1.20E−02 |
| | receptor | | | | | | | | | | |
| | agonist | | | | | | | | | | |
| fenoterol | adrenergic | 3.20 | 1.69E−03 | −5.33 | 5.76E−07 | −7.81 | 4.91E−13 | 3.98 | 1.43E−04 | −4.03 | 9.55E−05 |
| | receptor | | | | | | | | | | |
| | agonist | | | | | | | | | | |
| IBC-293 | hydroxycarboxylic | 6.70 | 4.22E−10 | −4.39 | 3.47E−05 | −7.75 | 7.38E−13 | −0.94 | 1.61E−01 | −7.54 | 2.12E−12 |
| | acid | | | | | | | | | | |
| | receptor | | | | | | | | | | |
| | agonist | | | | | | | | | | |
| cyclopentolate | acetylcholine | 5.44 | 3.33E−07 | −6.97 | 9.05E−11 | −7.53 | 3.18E−12 | −1.87 | 4.03E−02 | −6.93 | 8.85E−11 |
| | receptor | | | | | | | | | | |
| | antagonist | | | | | | | | | | |
| SQ-22536 | adenylyl | 2.41 | 1.34E−02 | −4.67 | 1.13E−05 | −7.53 | 3.18E−12 | −0.39 | 2.73E−01 | 4.37 | 2.63E−05 |
| | cyclase | | | | | | | | | | |
| | inhibitor | | | | | | | | | | |
| UBP-296 | glutamate | 7.21 | 1.88E−11 | −5.69 | 1.05E−07 | −7.45 | 5.13E−12 | −0.83 | 1.82E−01 | −6.80 | 2.05E−10 |
| | receptor | | | | | | | | | | |
| | antagonist | | | | | | | | | | |
| ciproxifan | histamine | 4.69 | 9.30E−06 | −0.17 | 3.39E−01 | −2.15 | 1.95E−02 | −8.70 | 5.04E−16 | −5.31 | 4.99E−07 |
| | receptor | | | | | | | | | | |
| | antagonist | | | | | | | | | | |
| neostigmine | acetylcholinesterase | 8.09 | 5.32E−14 | −1.63 | 6.85E−02 | −4.38 | 2.54E−05 | −8.60 | 1.16E−15 | −10.26 | 5.59E−22 |
| | inhibitor | | | | | | | | | | |
| FIT | opioid receptor | 6.94 | 1.07E−10 | 0.88 | 1.87E−01 | −4.10 | 7.10E−05 | −8.05 | 5.14E−14 | −7.17 | 2.06E−11 |
| | agonist | | | | | | | | | | |
| quinpirol-(—) | dopamine | 5.71 | 8.77E−08 | −1.04 | 1.57E−01 | −3.75 | 2.49E−04 | −7.38 | 5.19E−12 | −6.25 | 4.85E−09 |
| | receptor | | | | | | | | | | |
| | agonist | | | | | | | | | | |
| clopidogrel | purinergic | 7.21 | 1.88E−11 | 2.23 | 2.23E−02 | −2.23 | 1.67E−02 | −7.21 | 1.49E−11 | −6.86 | 1.35E−10 |
| | receptor | | | | | | | | | | |
| | antagonist | | | | | | | | | | |
| pinacidil | ATP | 2.32 | 1.65E−02 | 2.41 | 1.54E−02 | −0.36 | 2.42E−01 | −7.13 | 2.29E−11 | −4.01 | 1.01E−04 |
| | channel | | | | | | | | | | |
| | activator, | | | | | | | | | | |
| | potassium | | | | | | | | | | |
| | channel | | | | | | | | | | |
| | activator | | | | | | | | | | |
| atorvastatin | HMGCR | 2.36 | 1.49E−02 | 2.73 | 6.93E−03 | 0.29 | 2.53E−01 | −7.03 | 4.30E−11 | −2.26 | 1.49E−02 |
| | inhibitor | | | | | | | | | | |
| DMP-543 | acetylcholine | 6.60 | 7.26E−10 | −0.63 | 2.39E−01 | −5.85 | 3.62E−08 | −6.83 | 1.49E−10 | −9.12 | 1.37E−17 |
| | release | | | | | | | | | | |
| | enhancer | | | | | | | | | | |
| emetine | protein | 5.76 | 6.82E−08 | 4.41 | 3.25E−05 | −1.13 | 1.11E−01 | −6.80 | 1.69E−10 | −3.43 | 6.82E−04 |
| | synthesis | | | | | | | | | | |
| | inhibitor | | | | | | | | | | |
| FH-535 | PPAR | −0.54 | 2.45E−01 | −0.25 | 3.23E−01 | −0.72 | 1.76E−01 | −6.54 | 7.91E−10 | −1.62 | 5.13E−02 |
| | receptor | | | | | | | | | | |
| | antagonist, | | | | | | | | | | |
| | WNT | | | | | | | | | | |
| | signaling | | | | | | | | | | |
| | inhibitor | | | | | | | | | | |
| neostigmine | acetylcholinesterase | 8.09 | 5.32E−14 | −1.63 | 6.85E−02 | −4.38 | 2.54E−05 | −8.60 | 1.16E−15 | −10.26 | 5.59E−22 |
| | inhibitor | | | | | | | | | | |
| DMP-543 | acetylcholine | 6.60 | 7.26E−10 | −0.63 | 2.39E−01 | −5.85 | 3.62E−08 | −6.83 | 1.49E−10 | −9.12 | 1.37E−17 |
| | release | | | | | | | | | | |
| | enhancer | | | | | | | | | | |
| piperidolate | acetylcholine | 8.63 | 1.42E−15 | −3.49 | 8.19E−04 | −5.53 | 1.75E−07 | −5.16 | 1.11E−06 | −9.12 | 1.37E−17 |
| | receptor | | | | | | | | | | |
| | antagonist | | | | | | | | | | |
| FK-888 | tachykinin | 7.84 | 3.06E−13 | −4.36 | 3.95E−05 | −7.95 | 2.32E−13 | −1.94 | 3.54E−02 | −9.01 | 2.84E−17 |
| | antagonist | | | | | | | | | | |
| PNU-22394 | serotonin | 7.60 | 1.47E−12 | −6.08 | 1.39E−08 | −7.92 | 2.32E−13 | 0.47 | 2.55E−01 | −8.56 | 1.28E−15 |
| | receptor | | | | | | | | | | |
| | agonist | | | | | | | | | | |
| altanserin | serotonin | 7.87 | 2.54E−13 | −3.22 | 1.81E−03 | −7.22 | 1.87E−11 | −4.50 | 1.85E−05 | −7.91 | 1.99E−13 |
| | receptor | | | | | | | | | | |
| | antagonist | | | | | | | | | | |
| gamma- | cyclooxygenase | 5.48 | 2.83E−07 | 1.26 | 1.18E−01 | −2.95 | 2.88E−03 | −4.54 | 1.59E−05 | −7.89 | 2.16E−13 |
| linolenic-acid | inhibitor, | | | | | | | | | | |
| | prostanoid | | | | | | | | | | |
| | receptor | | | | | | | | | | |
| | agonist | | | | | | | | | | |
| alpha-linolenic- | omega 3 | 6.54 | 1.03E−09 | −1.25 | 1.19E−01 | −4.70 | 7.16E−06 | −2.80 | 5.23E−03 | −7.88 | 2.18E−13 |
| acid | fatty acid | | | | | | | | | | |
| | stimulant | | | | | | | | | | |
| | adrenergic | | | | | | | | | | |
| salmeterol | receptor | 7.89 | 2.38E−13 | −0.11 | 3.51E−01 | −3.28 | 1.11E−03 | −5.45 | 2.89E−07 | −7.87 | 2.19E−13 |
| | agonist | | | | | | | | | | |
| DR-2313 | PARP | 8.16 | 3.73E−14 | −0.49 | 2.68E−01 | −6.61 | 6.66 [symbol] | −2.80 | 5.20E−03 | −7.77 | 4.40E−13 |
| | inhibitor | | | | | | | | | | |

[symbol]

indicates data missing or illegible when filed

The DrugBank name, EMUDRA normalized score against LINCS drug signatures evaluated in NPCs (higher is better, negative is a detrimental drug), mechanism of action, p-value, and adjusted q-value fit with the gene differential expression signature for each AD subtype are provided for each of the top 10 drugs per subtype class. A drug must have a negative normalized score and significant (<0.05) q-value to be beneficial drug for a particular subtype; positive normalized scores are detrimental.

### Figures

FIG. 1: Identification of five stable molecular subtypes of AD. A-B) WSCNA clustering dendrogram and topological overlap matrix (TOM) heatmap, showing three major classes (A, B and C) and five subtypes annotated as A, B1, B2 C1 and C2, corresponding to the respective greyscale clusters. C) Number of samples in each subtype, control (CDR=0) and MCI (CDR=0.5). D) Gene expression profiles of all the samples in the PHG from the MSBB-AD cohort. The samples on the columns are grouped by the subtypes and the genes on the rows are grouped by the WINA modules. E) Change in mean expression level of various gene pathways for each AD subtype in comparison with the normal control samples. AD related pathways, representing differential expression from previous AD studies, are derived from the MSigDB. Sets are grouped by major area of biological activity.

FIG. 2: Mean values of several clinical and pathologic traits across AD subtypes. A) Bar plots of mean clinical dementia rating, brain bank score, amyloid plaque number, tauNFT scores (measured in the entorhinal cortex, the medial superior temporal cortex, and the medial frontal cortex), APOE4 allele count, and APOE2 allele count across five subtypes, control, and MCI. B) Stacked bar chart of inferred biological sex from transcriptomic data for all the PHG samples, across five subtypes, control, and MCI. C) Natural log-transformed p-values from the Kruskal-Wallis analysis of variance test of clinical, pathologic, and demographic variables. Significant tests are greater than −3.0, which corresponds to an alpha of 0.05.

FIG. 3: MEGENA and Bayesian causal network based key drivers of the AD subtypes. A-B) Top down- and upregulated MEGENA key drivers for each subtype plotted in its location in the network. Color of a node represents subtype (ties resolved; described below), while size of a node corresponds to the total number of genes in the 2-hop network neighborhood around the gene which are differentially expressed. Some genes are drivers for more than one subtype and ties are resolved by coloring the node corresponding to the subtype with the smallest signature so as to preserve faint signals. C) Heatmap of the top 20 down- and upregulated MEGENA key drivers for each AD subtype, where size of the node represents KNR natural log p-value (larger is more significant). D) Heatmap of the top 20 down- and upregulated Bayesian causal network key drivers for each AD subtype. E) AD subtype key drivers in the MEGENA network supported by gene expression changes in other brain regions, which the region of overlap listed.

FIG. 4: Cell type specific changes within each MSBB-AD subtype. A) Mean change in cell type proportion in each AD subtype, computed by averaging SPVs for the samples after cell type deconvolution by BRETIGEA. B-F) Vector addition of squared expression levels of AD subtype key regulators (up- and down-regulated) across 5 different cell types in a brain cell type specific sequencing experiment (Zhang et al. 2015).

FIG. 5: Identification of the AD subtypes in the ROSMAP cohort. A) WSCNA Clustering dendrogram and TOM similarity heatmap of the AD samples in the ROSMAP cohort. B) Heatmap of gene expression across all the ROSMAP AD and control samples. Genes are organized by the WINA gene modules identified in the gene expression data of the PHG in the MSBB cohort. C) Correlations between the MSBB and ROSMAP subtypes. First, mean gene expression across each AD subtype is computed for each gene, resulting in a 10 by 13,982 matrix of expression levels for all the 10 subtypes in the two cohorts (lowly-expressed genes are excluded). Pearson correlation is then computed between each pair of subtypes, resulting in a 10 by 10 correlation matrix. D) Cell type SPV mean by ROSMAP AD subtype class, inferred as in A,B, for MMSE-normalized data. E) APOE genotype proportion by ROSMAP subtype. F) Mean prediction accuracy of prediction of the ROSMAP subtypes by a classifier trained from the subtypes and the data from the RNA-seq data from the PHG in the MSBB cohort. Random forest (RF) classifiers are trained based on different numbers of key regulators of the AD subtype classes identified in the MSBB cohort. The subtypes for each AD case in the ROSMAP cohort are independently determined as described in FIG. 7 and are thus used as ground truth for evaluating the prediction by the predictors trained by the MSBB-AD data. G) Bar plots of the association of clinical and pathologic phenotypes with the predicted subtypes of each ROSMAP sample.

FIG. 6: Matching existing AD Mouse Models to the MSBB-AD subtypes. A) GSEA enrichment of differential expression signatures of the identified AD subtypes (up- and downregulated) for the gene signatures of the AD mouse models. Positive scores indicate strong consistency. B) Gene expression of the top subtype key regulators across the mouse models, with significant DEGs shown.

FIG. 7: Genomic CNV distribution in the two cohorts (MSBB and ROSMAP). Track 0: Human genome cytoband. Track 1: Deletions in ROSMAP. Track 2: Duplications in the ROSMAP. Track 3: multi-allelic CNVs in ROSMAP. Track 4: AD-specific CNVs in the ROSMAP. Track 5: Deletions in MSBB. Track 6: Duplications in MSBB. Track 7: multi-allelic CNVs in MSBB. Track 8: AD-specific CNVs in MSBB. Orange and blue lines represent deletion and duplication, respectively. Green lines represent multi-allelic CNVs.

FIG. 8: Overall features of the CNVs identified in MSBB and ROSMAP, including composition of CNV types, site frequency spectrum (SFS). A) Pie chart of the CNV composition in each cohort. B) CNV sharing pattern across the two cohorts. The CNV proportion in each category is based on the boundary of each cohort separately. The overlapping criteria is defined as the reciprocal overlap ratio larger than 0.5. C) SFS of deletions and duplications in the MSBB and ROSMAP cohorts.

FIG. 9: Comparison of the CNV sets in three clinical diagnostic groups (NL, MCI, and AD) in the two cohorts (MSBB and ROSMAP). A) Intersection of the CNV sets in three different diagnostic groups in each cohort. The numbers are defined by comparing different diagnostic groups in the same cohort. B) Illustration of the concept of group-specific CNVs. The pink, orange, and green shadow regions represent the AD-specific, MCI-specific, and NL-specific CNV sets. All the samples in the two cohorts are considered here. C) Intersection of the diagnostic group-specific CNV sets in MSBB and ROSMAP. The numbers are based on the cross-cohort comparison. D) SFS of AD-specific deletions and duplications. DEL and DUP represent deletion and duplication, respectively.

FIG. 10: Functional analysis of AD-, MCI-, and NL-specific CNV genes. CNV genes are the genes whose genomic locations overlap with a given CNV. A) AD-specific CNV genes are enriched for cellular glucuronidation, neuron projection, uronic acid metabolic process, extrinsic component of plasma membrane, synapse, catenin complex, and multicellular organismal signaling. B) Genes whose genomic locations overlap with multiple AD-specific CNVs are enriched for neuron development, neuron recognition, neuron differentiation, cell projection organization, neurogenesis, axon, and neuron projection. C) MCI-specific CNV genes are enriched for ligase activity forming carbon-sulfur bonds. D) NL-specific CNV genes are enriched for immunoglobulin complex. E) Circos plot of the sixty-four conserved AD-specific CNVs in MSBB and ROSMAP. The outer track 1 represents the genomic locations of the sixty-four conserved AD-specific CNVs, while the outer track 2 represents the genes whose genomic locations overlap these 64 CNVs. The inner track 1 represents the genomic location of the APP duplication region. F) The twenty-nine AD-specific CNVs encompassing the APP duplication region illustrated in the UCSC genome browser track. The light blue shade represents the location of the APP gene.

FIG. 11: The AUC-ROC Curve of different machine learning methods in predicting AD subtypes based on blood monocyte transcriptome. For each method, 200 features were selected by random forest and 10-fold cross-validation experiments were conducted to evaluate the classification performance.

### AD Cohorts

The present disclosure employs AD cohorts of RNA-seq data: the Mount Sinai Brain Bank (MSBB) study and the Religious Orders Study-Memory and Aging Project (ROSMAP). The MSBB-AD cohort includes RNA expression data in the following four different brain regions: Frontal Pole (FP, Brodmann area 10; n=265 with 187 AD cases), Superior Temporal Gyrus (STG, Brodmann area 22; n=240 with 174 AD cases), Parahippocampal Gyrus (PHG, Brodmann area 36; n=215 with 151 AD cases) and the Inferior Frontal Gyrus (IFG, Brodmann area 44; n=222 with 157 AD cases). Clinical phenotypes for each subject are also collected including age, race, sex, hypoxia-induced encephalopathy (HIE) score, cognitive function scores, CDR, age of onset and death, and pathologic findings of Tau and amyloid on biopsy. This cohort was specifically selected to include cases with either no neuropathology, or only neuropathological lesions diagnostic of AD. Cases with mixed neuropathology, e.g., AD and cerebrovascular disease, AD with Lewy bodies, etc. were specifically excluded from the study cohort. Controls were defined as those presenting with no cognitive impairment (i.e., CDR=0) and no significant neuritic plaque or neurofibrillary tangle involvement.

The Religious Orders Study-Memory and Aging Project (ROSMAP) includes whole transcriptome RNA-seq data of the dorsolateral prefrontal cortex (DLPFC) from 615 subjects including those with AD (n=391), mild cognitive impairment (n=64), and non-demented controls (n=160) determined by a CERAD pathology score of Definite AD or Probable AD. Clinical and pathologic phenotypes, as well as demographic information, were also collected as well for each sample including MMSE scores (at time of diagnosis and last known), CERAD score, Braak score, cognitive score, APOE genotype, age of death, age at diagnosis, post-mortem interval, gender, race, education level, and if the subject was Spanish-speaking

### Clustering Algorithm Evaluation and Cluster Stability Determination

On the MSBB-AD cohort data, rounds of bootstrapped reclustering were performed using each of the four clustering algorithms, with 20% of the samples and genes withheld per round. The rate at which pairs of samples shared the same cluster were calculated across all 50 bootstrapping rounds (e.g., a pair of samples clustered together in 35 out of 50 bootstrapped clustering rounds would have a rate of 70%), defined as the pairwise sample reclustering rate. The average pairwise sample reclustering rate were then calculated for all pairs of samples within the sample clusters identified by each algorithm, as well as the average rate of same-sized clusters drawn from a distribution of 100,000 random pairs of samples. These average rates were termed the cluster stability rate and the null cluster stability rate, respectively. Then a calculation was performed to determine the empirical likelihood that the cluster stability rate and the null cluster stability rate are the same, under the binomial distribution. Using this method, a specific subtype grouping is considered a putative subtype if its empirically-adjusted p-value is less than 0.05.

### RNA-Seq Data Normalization

MSBB-AD RNA-seq data was processed with the STAR aligner and normalized using mixed model correction for batch effect, RNA integrity number, rRNA rate, exonic RNA rate, post-mortem interval (PMI), age of death (AOD), inferred race, and inferred sex. Label swaps were inferred and corrected or removed if resolution was not possible.

To remove the disease stage effect, CDR is corrected in the MSBB-AD gene expression data through linear model normalization. This was verified by performing a second round of linear model fitting between CDR and gene expression which showed no correlation significantly differentially expressed genes were observed between patients with and without dementia across all brain regions in the MSBB-AD.

ROSMAP DLPFC RNA-seq data was also normalized for age of death, gender, batch, RIN, and PMI using mixed model correction. Data were then subsequently normalized for last known MMSE score using a linear model, and no genes are shown subsequently to have a correlation with MMSE (R²=0).

### Differential Gene Expression Analysis of AD Subtypes

Differential gene expression (DEG) analysis was performed to determine the molecular signatures of each of the AD subtypes compared with non-demented (CDR=0) controls, starting with the RNA-seq counts per million (CPM) data as input. The analysis was carried out separately for each comparison. Log-scaled (base 2) gene CPMs from samples in the comparison were first fit to a linear model using the lmfit( ) provided by the limma R package before contrasts were fit. Empirical Bayes statistics for differential expression were then calculated using the eBayes( ) R function, followed by the topTable( ) R function to output significant DEGs. P-values were adjusted by qvalues provided by the qvalue Bioconductor package, using default parameters.

### Clustering Algorithms Used in the Establishment of Putative AD Subtypes

WSCNA identifies sample clusters by analyzing gene expression level correlations between pairs of samples to build a sample correlation network, which is then used to calculate topological overlap (TOM) score that can be used to cluster similar samples together via k-means clustering. WSCNA extends the WINA algorithm to samples by transposing the input matrix so that sample-sample correlations are compared. Note that gene expression data is standardized to z-scores so that expression differences do not inflate the correlation metric.

### Network-Based Key Network Regulator Gene Analysis of Molecular Subtype Signatures

Key driver analysis (“KDA”) (McKenzie et al. 2017) was applied to the multiscale embedded gene expression network analysis (MEGENA) network generated from parahippocampal gyrus data in the MSBB. KDA first generates a subnetwork NG, defined as the set of nodes in N that are no more than h layers away from the nodes in G, and then searches the h-layer neighborhood (h=1, . . . , H) for each gene in NG (HLN_{g,h}) for the optimal h*, such that

ESₕ*=max(ES_{h,g})∀g∈N_{g,h}∈{1, . . . ,H}

where, ES_{h,g} is the computed enrichment statistic for HLN_{g,h}. This results in a list of predicted key network regulatory hub genes that may alter the expression pattern of its surrounding nodes and result in the DEG pattern observed.

### Machine Learning Subtype Classifier Across Other Cohorts

The inventors developed a random forest (RF) model to classify samples into each AD subtype using the MSBB-AD PHG brain region data for training and then validated this model on the ROSMAP data. All RF models were built using the scipy Python library, with initial parameters of 300 decision trees and a maximum tree depth of 8. Before model creation, both datasets are first corrected for cohort effect between MSBB-AD and ROSMAP, using the ComBat program, to reduce technical differences between studies. Classifier creation was divided into three steps: feature selection, model training, and model validation. First, for the feature selection step, different numbers of top key network regulator genes were selected as features from each subtype (n=1 to 80 features per subtype, total: 5-400 features). Second multiple RF models were trained to predict subtype classification within the MSBB-AD (PHG) cohort and to evaluate the model accuracy using leave-one-out cross-validation between the predicted and observed subtypes. In the model training step, a RF model is created on all AD patients' PHG samples in MSBB-AD using only the top-performing features identified in the previous step. Finally, for the model validation step, the RF model created from the MSBB-AD data is applied to the ROSMAP data, and model accuracy is evaluated by comparing the predicted ROSMAP subtypes from the RF model and the observed ROSMAP subtypes from network-based clustering analysis. The number of features used in the RF model are increased until maximum validation accuracy is achieved, and the top-performing set of features from this model are retained.

### Bayesian Causal Network Construction

A Bayesian causal network was constructed by integrating genome-wide gene expression, SNP genotype, and known transcription factor (TF)-target relationships in the PHG in the MSBB-AD cohort. First, expression quantitative trait loci (eQTLs) are computed and then a formal statistical causal inference test (CIT) is employed to infer the causal probability between gene pairs associated with the same eQTL. The causal relationships inferred are used, together with TF-target relationships from the ENCODE project, as structural priors for building a causal gene regulatory network from the gene expression data through a Monte Carlo Markov Chain (MCMC) simulation-based procedure. A network averaging strategy was followed, in which 1,000 networks are generated from the MCMC procedure starting with different random structure and links that shared by more than 30% of the networks are used to define a final consensus network structure. To ensure the consensus network is a directed acyclic graph, an iterative de-loop procedure was conducted, removing the most-weakly supported link of all links involved in any loop. Key Driver Analysis (KDA) was performed on the consensus Bayesian network to identify key network regulatory genes which can potentially regulate numerous downstream nodes. (Zhang et al. 2013; MacKenzie et al. 2017).

### Cell-Type Proportion Analysis and Cell-Type Normalization

To estimate the cell-type proportion of bulk tissue RNA-seq data, a cell-type deconvolution was performed on each sample using the brain cell type marker signatures provided by the BRETIGEA R package. One thousand marker genes per cell type were used from the human brain cell marker gene set (neurons, endothelials, oligodendrocytes, microglia, and astrocytes) to generate all surrogate cell-type proportion (SPV) estimates, except for oligodendrocyte precursor cells which only had 500 marker genes available. Normalization of the bulk RNA-seq by brain cell type was also performed by BRETIGEA, using the default parameters and the calculated SPV values from the previous step.

### Cell-Type Specificity Plots

To generate cell-type specificity plots, using the mean cell-type gene expression levels from (Zhang Y. et al. 2007), each squared expression value was plotted as a vector from the center on a polar coordinate system. The inventors then calculated the vector sum from each of the expression levels and multiplied the final result by a scaling parameter to create a final point as the estimate of the cell-type specificity of any gene under consideration.

### Molecular Subtyping by Gene Expression

The gene expression profiles of the blood monocytes were downloaded from the AD Knowledge Portal (Synapse ID: syn22024496). The peripheral blood mononuclear cells (PBMCs) were isolated based on the CD14+CD16−markers using the EasySep Human Monocyte Isolation Kit (Negative selection kit, Stemcell Technologies, 19359). Then the Live (BV510-) CD14+/CD16-cells were sorted on a BD Influx cell sorter. RNA sequencing libraries were prepared using SMART-seq2 protocols for cDNA preparation followed by Nextera XT DNA library preparation. The pooled cDNA libraries were sequenced by HiSeq 2500 and NovaSeq 6000 (Illumina).

RNA sequencing libraries were prepared from peripheral blood mononuclear cells based on the CD14+CD16−markers of AD individuals. Among the sequenced samples, 102 of them have matched brain tissues with AD pathology in the ROSMAP database. These brain tissues could be broadly classified into two AD subtypes: the typical (n=57) and untypical (n=45) subtypes. The gene expressions were normalized via a linear model for different library size by TMM method of edgeR, and adjusted co-variates including MMSE, ExonicRate, SEX, study and Batch by a liner model. The resulting standardized expression levels of the different features were used as the input for the machine learning tools. Different parameters were evaluated during the feature selection and model training. Different features sizes (50, 100, 200 and 500) were chosen based on random forest important scores or differentially expressed genes (DEGs, p-value<0.05) between AD subtypes. During the model training, the whole dataset was split into training and testing datasets based on the rules of the K fold cross-validation. Three K values (5, 10, 15) were used for evaluation. For each fold of the cross-validation experiment, nine methods were used to fit the training dataset and the accuracy of the testing dataset was calculated. Finally, the mean accuracy and the standard deviation were calculated from the K-fold experiments. Nine different machine learning methods were implemented with equal weights for each classifier, including random forest, AdaBoost, logistic regression, decision tree, nearest neighbors (KNN), support vector machines (SVM), naïve Bayes, multi-layer perceptron, and an Ensemble method.

### Whole-Genome Sequencing Data

The whole-genome sequencing data in the MSBB cohort are available at the AMP-AD knowledge portal (synapse ID: syn10901600). The WGS data in MSBB were generated from 353 individuals, of which 341 had clinical and pathological data (i.e., age of death, Clinical Dementia Rate (CDR), Plaque Mean, CERAD score, and Braak stage score (bbscore)). The 284 North American Caucasian samples were used. Subjects with CDR scores larger than 0.5 were classified as AD, those with CDR equal to 0.5 were classified as mild cognitive impairment (MCI), and those with CDR equal to zero were classified as healthy controls (NL). Under this classification scheme, there are 224 AD cases, 27 MCI cases, and 33 NL cases. The mean sequencing depth of all samples is 36.58X. There is no significant difference in sequencing depth among the three groups.

There are 1,200 individuals with whole-genome sequencing data in the ROSMAP cohort (synapse ID: syn10901595). Outliers that contained more than 6,000 deletions or 1000 duplications in the individual scanning stage and other dementia cases were excluded. The filtering process identified 71 outliers. Non-Caucasian samples were also excluded and 1,127 Caucasian samples were used in the afterward analysis. The subjects were classified into three diagnostic groups based on their final Clinical Consensus Diagnosis (AD: 4 or 5; MCI: 2 or 3; NL: 1). Under this definition, there are 477 AD, 285 MCI, and 365 NL subjects.

The RNA-seq based transcriptomic data in MSBB are also available at the AMP-AD portal (synapse ID: syn3157743). The samples were extracted from the BM-10, BM-22, BM-36, and BM-44 regions. Information about the MSBB samples and sequencing data can be found in a previous publication (Wang M. et al. 2018). RNA-seq data normalization and covariate correction were detailed in (Wang et al. 2020). RNA-seq data in MSBB were adjusted for covariates, including postmortem interval (PMI), RNA integrity number (RIN), race, age of death (AOD), batch effect, and sex.

The RNA-seq transcriptomic data in the DLPFC region of the ROSMAP cohort was downloaded from the AMP-AD portal (synapse ID: syn3388564). The read alignment, gene expression quantification, normalization, and covariate correction were performed using the same pipeline as the MSBB data. Briefly, the reads were mapped to human genome hg19 using the STAR aligner (v2.3.0e), and then gene-level expression was quantified by featureCounts (v1.6.3) based on Ensembl gene model GRCh37.70. Next, gene-level count data was normalized using the R/limma's voom function and subsequently corrected for known covariates, including sequencing batch, PMI, AOD, sex, and RIN by a mixed model.

1. A method for treating Alzheimer's Disease in a patient in need thereof, the method comprising detecting markers associated with at least one Alzheimer's Disease (AD) subtype in a biological sample from said patient, normalizing said marker levels, using a trained machine learning technique to provide a score for the AD subtype of said patient, comparing said score with a predetermined reference standard, determining said AD subtype, and providing a treatment for said AD subtype.

2. The method of claim 1, wherein said detecting markers is a Weighted Sample Correlation Network Analysis (WSCNA).

3. The method of claim 2, wherein said detecting identifies polynucleotide markers, polypeptide markers, or both.

4. The method of claim 3, wherein said polynucleotide markers are selected from the group consisting of: polynucleotide length, epigenetic markers, methylation levels, nucleotide sequence, copy number, single nucleotide polymorphisms, sequence expression levels, RNA expression, RNA stability, and sequence transpositions or translocations.

5. The method of claim 3, wherein said detecting identifies polypeptides, epitopes, or fragments.

6. The method of claim 2, wherein said biological sample is selected from the group consisting of: blood, cerebrospinal fluid, lymphatic tissue, cells, epithelial tissue, and adipose tissue.

7. The method of claim 2, wherein the at least one Alzheimer's Disease subtype is selected from the group consisting of: AD subtype A, AD subtype B1, AD subtype B2, AD subtype C1, and AD subtype C2.

8. The method of claim 2, comprising providing a therapeutically effective amount of a drug selected from the group consisting of: thioproperazine; nalbuphine; gabexate; mesoridazine; menadione; carbamazepine; diphenidol; epirizole; timolol; mestranol; naphazoline; hesperidin; ethisterone; amlodipine; amsacrine; febuxostat; famciclovir; ezetimibe; carbetocin; orphenadrine; hyoscyamine; amiodarone.hcl; erythromycin-ethylsuccinate; meclizine; dobutamine; phenazopyridine; spironolactone; meclofenamic-acid; parachorophenol; bemegride; ketorolac; and brinzolamide.

9. The method of claim 1, wherein the trained machine learning technique is selected from the group consisting of: Random Forest, hierarchical clustering, k-means clustering, MEGENA, Bayesian causal network, CNVnator, Pindel, MetaSV, Delly2, Quasipoisson regression, AdaBoost, logistic regression, decision tree, nearest neighbors (KNN), support vector machines (SVM), naïve Bayes, multi-layer perceptron, and Ensemble.

10. A computer-implemented method to predict an AD subtype of a subject, the method comprising:
obtaining data concerning specific characteristics of a biological sample collected from a subject;
providing said data as input to a trained machine learning technique, wherein the technique determines the AD subtype based on the data; wherein the AD subtype is any one of: AD subtype A, AD subtype B1, AD subtype B2, AD subtype C1, or AD subtype C2; and
obtaining, from the machine learning technique, the predicted AD subtype.

11. The method of claim 10, wherein said machine learning technique is a Weighted Sample Correlation Network Analysis (WSCNA).

12. The method of claim 10, wherein said biological sample is selected from the group consisting of: blood, cerebrospinal fluid, lymphatic tissue, cells, epithelial tissue, and adipose tissue.

13. The method of claim 10, wherein said data comprises information concerning polynucleotide markers, polypeptide markers, or both.

14. The method of claim 13, wherein said polynucleotide markers are selected from the group consisting of: sequence length, epigenetic markers, methylation levels, sequence code, copy number, single nucleotide polymorphisms, sequence expression levels, RNA expression, RNA stability, and sequence transpositions or translocations.

15. The method of claim 13, wherein said data comprises information concerning polypeptides, epitopes, or fragments.

16. The method of claim 10, wherein the trained machine learning technique is selected from the group consisting of: Random Forest, hierarchical clustering, k-means clustering, MEGENA, Bayesian causal network, CNVnator, Pindel, MetaSV, Delly2, Quasipoisson regression, AdaBoost, logistic regression, decision tree, nearest neighbors (KNN), support vector machines (SVM), naïve Bayes, multi-layer perceptron, and Ensemble

17. A method for treating Alzheimer's in a subject in need thereof, the method comprising: receiving the AD subtype of the subject, which has been obtained using the method of claim 11 and administering a therapeutically effective amount of drug for targeting the obtained AD subtype.

18. The method of claim 17, comprising administering a therapeutically effective amount of a drug selected from the group consisting of: thioproperazine; nalbuphine; gabexate; mesoridazine; menadione; carbamazepine; diphenidol; epirizole; timolol; mestranol; naphazoline; hesperidin; ethisterone; amlodipine; amsacrine; febuxostat; famciclovir; ezetimibe; carbetocin; orphenadrine; hyoscyamine; amiodarone.hcl; erythromycin-ethylsuccinate; meclizine; dobutamine; phenazopyridine; spironolactone; meclofenamic-acid; parachorophenol; bemegride; ketorolac; and brinzolamide.

19. The method of claim 2, further comprising using computer implemented method for identifying candidate compounds for use in treating an Alzheimer's Disease subtype, the method comprising:
obtaining data of drug induced signatures for candidate compounds and Alzheimer's Disease subtype signatures;
providing the data as input to a trained machine learning model, wherein the model is EDMURA; and obtaining from the model, the drug associated with an Alzheimer's Disease subtype.

20. The method of claim 11, further comprising using computer implemented method for identifying candidate compounds for use in treating an Alzheimer's Disease subtype, the method comprising:
obtaining data of drug induced signatures for candidate compounds and Alzheimer's Disease subtype signatures;
providing the data as input to a trained machine learning model, wherein the model is EDMURA; and obtaining from the model, the drug associated with an Alzheimer's Disease subtype.
